SEQUENCE LISTING (61 sequences)

SEQ ID NO: 1 - protein, 1471 aa, Artificial Sequence
  Synthetic
MKRPAATKKAGQAKKKKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSGRADALDDFDLDMLGSDALDDFDLDMLGSDALDDFDLDMLGSDALDDFDLDMLINCT

SEQ ID NO: 2 - protein, 1384 aa, Artificial Sequence
  Synthetic
MKRPAATKKAGQAKKKKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 62 aa, Artificial Sequence
  Synthetic
AAAGSGRADALDDFDLDMLGSDALDDFDLDMLGSDALDDFDLDMLGSDALDDFDLDMLINCT

SEQ ID NO: 4 - protein, 4 aa, Artificial Sequence
  Synthetic
GGGS

SEQ ID NO: 5 - protein, 5 aa, Artificial Sequence
  Synthetic
GGGGS

SEQ ID NO: 6 - protein, 473 aa, Artificial Sequence
  Synthetic
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 7 - protein, 130 aa, Artificial Sequence
  Synthetic
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 8 - protein, 181 aa, Artificial Sequence
  Synthetic
PSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISS

SEQ ID NO: 9 - protein, 124 aa, Artificial Sequence
  Synthetic
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 10 - RNA, 16 nt, Artificial Sequence
  Synthetic
guuuuagagcuaugcu

SEQ ID NO: 11 - RNA, 67 nt, Artificial Sequence
  Synthetic
agcauagcaaguuaaaauaaggcuaguccguuaucaacuugaaaaaguggcaccgagucggugcuuu

SEQ ID NO: 12 - RNA, 77 nt, Artificial Sequence
  Synthetic
guuuuagagcuagaaauagcaaguuaaaauaaggcuaguccguuaucaacuugaaaaaguggcaccgagucggugcu

SEQ ID NO: 13 - RNA, 82 nt, Artificial Sequence
  Synthetic
guuggaaccauucaaaacagcauagcaaguuaaaauaaggcuaguccguuaucaacuugaaaaaguggcaccgagucggugc

SEQ ID NO: 14 - RNA, 76 nt, Artificial Sequence
  Synthetic
guuuuagagcuagaaauagcaaguuaaaauaaggcuaguccguuaucaacuugaaaaaguggcaccgagucggugc

SEQ ID NO: 15 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 16 - RNA, 34 nt, Artificial Sequence
  Synthetic
ggccaacaugaggaucacccaugucugcagggcc

SEQ ID NO: 17 - DNA, 23 bp, Artificial Sequence
  Synthetic
  misc_feature (2)..(21): n is a, c, g, or t
gnnnnnnnnnnnnnnnnnnnngg

SEQ ID NO: 18 - DNA, 23 bp, Artificial Sequence
  Synthetic
  misc_feature (1)..(21): n is a, c, g, or t
nnnnnnnnnnnnnnnnnnnnngg

SEQ ID NO: 19 - DNA, 25 bp, Artificial Sequence
  Synthetic
  misc_feature (3)..(23): n is a, c, g, or t
ggnnnnnnnnnnnnnnnnnnnnngg

SEQ ID NO: 20 - protein, 18 aa, Artificial Sequence
  Synthetic
EGRGSLLTCGDVEENPGP

SEQ ID NO: 21 - protein, 19 aa, Artificial Sequence
  Synthetic
ATNFSLLKQAGDVEENPGP

SEQ ID NO: 22 - protein, 20 aa, Artificial Sequence
  Synthetic
QCTNYALLKLAGDVESNPGP

SEQ ID NO: 23 - protein, 22 aa, Artificial Sequence
  Synthetic
VKQTLNFDLLKLAGDVESNPGP

SEQ ID NO: 24 - DNA, 4152 bp, Artificial Sequence
  Synthetic
atgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 25 - DNA, 4414 bp, Artificial Sequence
  Synthetic
atgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacag

SEQ ID NO: 26 - DNA, 390 bp, Artificial Sequence
  Synthetic
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 27 - DNA, 1419 bp, Artificial Sequence
  Synthetic
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 28 - DNA, 187 bp, Artificial Sequence
  Synthetic
gcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacag

SEQ ID NO: 29 - DNA, 543 bp, Artificial Sequence
  Synthetic
ccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctct

SEQ ID NO: 30 - DNA, 372 bp, Artificial Sequence
  Synthetic
ggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 31 - DNA, 9043 bp, Artificial Sequence
  Synthetic
  misc_feature (1)..(34): First loxP site
  misc_feature (125 )..(928): Sequence encoding neomycin phosphotransferase for resistance to neomycin family antibiotics
  misc_feature (937)..(2190): Polyadenylation signal
  misc_feature (2218)..(2251): Second loxP site
  misc_feature (2306)..(6457): Codon-optimized dCas9 coding sequence
  misc_feature (2309)..(2356): NLS
  misc_feature (6512 )..(6532): NLS
  misc_feature (6533)..(6719): VP64
  misc_feature (6719)..(6781): T2A coding sequence with 5′ GSG
  misc_feature (6782)..(7171): MCP
  misc_feature (7226)..(7246): NLS
  misc_feature (7262)..(7804): p65
  misc_feature (7829)..(8200): HSF1
  misc_feature (8224)..(8820): Woodchuck hepatitis virus posttranscriptional regulatory element (WPRE)
ataacttcgtataatgtatgctatacgaagttattaggtccctcgacctgcaggaattgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaacccttaatataacttcgtataatgtatgctatacgaagttattaggtccctcgacctgcagcccaagctagtgcccgggaattcgctagggccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacaggcagtggagagggcagaggaagtctgctaacatgcggtgacgtcgaggagaatcctggcccaatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatggcaggcatg

SEQ ID NO: 32 - DNA, 3812 bp, Artificial Sequence
  Synthetic
  misc_feature (1)..(32): First rox site
  misc_feature (111)..(710): Sequence encoding puromycin-N- acetyltransferase for resistance to puromycin family antibiotics
  misc_feature (797)..(2338): Polyadenylation signal
  misc_feature (2363)..(2394): Second rox site
  misc_feature (2401)..(2640): First U6 promoter
  misc_feature (2641)..(2797): First guide RNA coding sequence
  misc_feature (2641)..(2660): n is a, c, g, or t
  misc_feature (2883)..(3122): Second U6 promoter
  misc_feature (3123)..(3279): Second guide RNA coding sequence
  misc_feature (3123)..(3142): n is a, c, g, or t
  misc_feature (3364)..(3603): Third U6 promoter
  misc_feature (3604)..(3760): Third guide RNA coding sequence
  misc_feature (3604)..(3623): n is a, c, g, or t
taactttaaataatgccaattatttaaagttacctgcaggacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcataacttcgtataatgtatgctatacgggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagccgacgctaggtcgtcagtcaaagtacgtacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttagatgttttactagccagatttttcctcctctcctgactactcccagtcatagctgtccctcttctcttatggagatccctcgaggacatgaggtcgtcgctgtaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgacactgggtcgtgatcgggtacctaactttaaataatgccaattatttaaagttagctagctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 33 - DNA, 3814 bp, Artificial Sequence
  Synthetic
  misc_feature (1)..(32): First rox site
  misc_feature (111)..(710): Sequence encoding puromycin-N- acetyltransferase for resistance to puromycin family antibiotics
  misc_feature (797)..(2338): Polyadenylation signal
  misc_feature (2363)..(2394): Second rox site
  misc_feature (2401)..(2640): First U6 promoter
  misc_feature (2642)..(2798): First Ttr guide RNA coding sequence
  misc_feature (2884)..(3123): Second U6 promoter
  misc_feature (3125)..(3281): Second Ttr guide RNA coding sequence
  misc_feature (3366)..(3605): Third U6 promoter
  misc_feature (3606)..(3762): Third Ttr guide RNA coding sequence
taactttaaataatgccaattatttaaagttacctgcaggacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcataacttcgtataatgtatgctatacgggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagccgacgctaggtcgtcagtcaaagtacgtacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttagatgttttactagccagatttttcctcctctcctgactactcccagtcatagctgtccctcttctcttatggagatccctcgaggacatgaggtcgtcgctgtaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgacactgggtcgtgatcgggtacctaactttaaataatgccaattatttaaagttagctagctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacggttgccctctttcccaagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgtcagactcaaaggtgcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaataagtagtcttactcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 34 - DNA, 20 bp, Mus musculus
acggttgccctctttcccaa

SEQ ID NO: 35 - DNA, 20 bp, Mus musculus
actgtcagactcaaaggtgc

SEQ ID NO: 36 - DNA, 20 bp, Mus musculus
gacaataagtagtcttactc

SEQ ID NO: 37 - RNA, 157 nt, Artificial Sequence
  Synthetic
acgguugcccucuuucccaaguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 38 - RNA, 157 nt, Artificial Sequence
  Synthetic
acugucagacucaaaggugcguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 39 - RNA, 157 nt, Artificial Sequence
  Synthetic
gacaauaaguagucuuacucguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 40 - RNA, 137 nt, Artificial Sequence
  Synthetic
guuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 41 - RNA, 20 nt, Artificial Sequence
  Synthetic
acgguugcccucuuucccaa

SEQ ID NO: 42 - RNA, 20 nt, Artificial Sequence
  Synthetic
acugucagacucaaaggugc

SEQ ID NO: 43 - RNA, 20 nt, Artificial Sequence
  Synthetic
gacaauaaguagucuuacuc

SEQ ID NO: 44 - protein, 1965 aa, Artificial Sequence
  Synthetic
MKRPAATKKAGQAKKKKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSGRADALDDFDLDMLGSDALDDFDLDMLGSDALDDFDLDMLGSDALDDFDLDMLINCTGSGEGRGSLLTCGDVEENPGPMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 45 - RNA, 157 nt, Artificial Sequence
  Synthetic
  misc_feature (1)..(20): n is a, c, g, or u
nnnnnnnnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 46 - DNA, 5895 bp, Artificial Sequence
  Synthetic
atgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacaggcagtggagagggcagaggaagtctgctaacatgcggtgacgtcgaggagaatcctggcccaatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 47 - DNA, 1412 bp, Artificial Sequence
  Synthetic
  misc_feature (241)..(260): n is a, c, g, or t
  misc_feature (723)..(742): n is a, c, g, or t
  misc_feature (1204)..(1223): n is a, c, g, or t
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 48 - DNA, 1414 bp, Artificial Sequence
  Synthetic
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacggttgccctctttcccaagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgtcagactcaaaggtgcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaataagtagtcttactcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 49 - DNA, 1490 bp, Artificial Sequence
  Synthetic
  misc_feature (28)..(267): hU6 promoter
  misc_feature (269)..(288): Guide1
  misc_feature (289)..(425): SAM Tracr
  misc_feature (426)..(477): Extended terminator
  misc_feature (511)..(750): hU6 promoter
  misc_feature (752)..(771): Guide2
  misc_feature (772)..(908): SAM Tracr
  misc_feature (909)..(960): Extended terminator
  misc_feature (993)..(1232): hU6 promoter
  misc_feature (1233)..(1252): Guide3
  misc_feature (1253)..(1389): SAM Tracr
  misc_feature (1390)..(1441): Extended terminator
gctagccataagactcggccttagaactttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacggttgccctctttcccaagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgtcagactcaaaggtgcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaataagtagtcttactcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttggtcacccagtgaggaagctaggacagacctaggacggttgcctgcagg

SEQ ID NO: 50 - RNA, 72 nt, Artificial Sequence
  Synthetic
aaacagcauagcaaguuaaaauaaggcuaguccguuaucaacuugaaaaaguggcaccgagucggugcuuuu

SEQ ID NO: 51 - RNA, 82 nt, Artificial Sequence; identical to SEQ ID NO: 13
  Synthetic

SEQ ID NO: 52 - RNA, 83 nt, Artificial Sequence
  Synthetic
guuuuagagcuagaaauagcaaguuaaaauaaggcuaguccguuaucaacuugaaaaaguggcaccgagucggugcuuuuuuu

SEQ ID NO: 53 - RNA, 80 nt, Artificial Sequence
  Synthetic
guuuuagagcuagaaauagcaaguuaaaauaaggcuaguccguuaucaacuugaaaaaguggcaccgagucggugcuuuu

SEQ ID NO: 54 - RNA, 92 nt, Artificial Sequence
  Synthetic
guuuaagagcuaugcuggaaacagcauagcaaguuuaaauaaggcuaguccguuaucaacuugaaaaaguggcaccgagucggugcuuuuuu

SEQ ID NO: 55 - RNA, 159 nt, Artificial Sequence
  Synthetic
gacaauaaguagucuuacucguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuu

SEQ ID NO: 56 - RNA, 139 nt, Artificial Sequence
  Synthetic
guuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuu

SEQ ID NO: 57 - RNA, 159 nt, Artificial Sequence
  Synthetic
  misc_feature (1)..(20): n is a, c, g, or u
nnnnnnnnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuu

SEQ ID NO: 58 - protein, 7 aa, Artificial Sequence
  Synthetic
PKKKRKV

SEQ ID NO: 59 - protein, 7 aa, Artificial Sequence
  Synthetic
PKKKRRV

SEQ ID NO: 60 - protein, 16 aa, Artificial Sequence
  Synthetic
KRPAATKKAGQAKKKK

SEQ ID NO: 61 - RNA, 5895 nt, Artificial Sequence
  Synthetic
augaaaaggccggcggccacgaaaaaggccggccaggcaaaaaagaaaaaggacaagaaguacagcaucggccuggccaucggcaccaacucugugggcugggccgugaucaccgacgaguacaaggugcccagcaagaaauucaaggugcugggcaacaccgaccggcacagcaucaagaagaaccugaucggagcccugcuguucgacagcggcgaaacagccgaggccacccggcugaagagaaccgccagaagaagauacaccagacggaagaaccggaucugcuaucugcaagagaucuucagcaacgagauggccaagguggacgacagcuucuuccacagacuggaagaguccuuccugguggaagaggauaagaagcacgagcggcaccccaucuucggcaacaucguggacgagguggccuaccacgagaaguaccccaccaucuaccaccugagaaagaaacugguggacagcaccgacaaggccgaccugcggcugaucuaucuggcccuggcccacaugaucaaguuccggggccacuuccugaucgagggcgaccugaaccccgacaacagcgacguggacaagcuguucauccagcuggugcagaccuacaaccagcuguucgaggaaaaccccaucaacgccagcggcguggacgccaaggccauccugucugccagacugagcaagagcagacggcuggaaaaucugaucgcccagcugcccggcgagaagaagaauggccuguucggcaaccugauugcccugagccugggccugacccccaacuucaagagcaacuucgaccuggccgaggaugccaaacugcagcugagcaaggacaccuacgacgacgaccuggacaaccugcuggcccagaucggcgaccaguacgccgaccuguuucuggccgccaagaaccuguccgacgccauccugcugagcgacauccugagagugaacaccgagaucaccaaggccccccugagcgccucuaugaucaagagauacgacgagcaccaccaggaccugacccugcugaaagcucucgugcggcagcagcugccugagaaguacaaagagauuuucuucgaccagagcaagaacggcuacgccggcuacauugacggcggagccagccaggaagaguucuacaaguucaucaagcccauccuggaaaagauggacggcaccgaggaacugcucgugaagcugaacagagaggaccugcugcggaagcagcggaccuucgacaacggcagcaucccccaccagauccaccugggagagcugcacgccauucugcggcggcaggaagauuuuuacccauuccugaaggacaaccgggaaaagaucgagaagauccugaccuuccgcauccccuacuacgugggcccucuggccaggggaaacagcagauucgccuggaugaccagaaagagcgaggaaaccaucacccccuggaacuucgaggaagugguggacaagggcgcuuccgcccagagcuucaucgagcggaugaccaacuucgauaagaaccugcccaacgagaaggugcugcccaagcacagccugcuguacgaguacuucaccguguauaacgagcugaccaaagugaaauacgugaccgagggaaugagaaagcccgccuuccugagcggcgagcagaaaaaggccaucguggaccugcuguucaagaccaaccggaaagugaccgugaagcagcugaaagaggacuacuucaagaaaaucgagugcuucgacuccguggaaaucuccggcguggaagaucgguucaacgccucccugggcacauaccacgaucugcugaaaauuaucaaggacaaggacuuccuggacaaugaggaaaacgaggacauucuggaagauaucgugcugacccugacacuguuugaggacagagagaugaucgaggaacggcugaaaaccuaugcccaccuguucgacgacaaagugaugaagcagcugaagcggcggagauacaccggcuggggcaggcugagccggaagcugaucaacggcauccgggacaagcaguccggcaagacaauccuggauuuccugaaguccgacggcuucgccaacagaaacuucaugcagcugauccacgacgacagccugaccuuuaaagaggacauccagaaagcccagguguccggccagggcgauagccugcacgagcacauugccaaucuggccggcagccccgccauuaagaagggcauccugcagacagugaaggugguggacgagcucgugaaagugaugggccggcacaagcccgagaacaucgugaucgaaauggccagagagaaccagaccacccagaagggacagaagaacagccgcgagagaaugaagcggaucgaagagggcaucaaagagcugggcagccagauccugaaagaacaccccguggaaaacacccagcugcagaacgagaagcuguaccuguacuaccugcagaaugggcgggauauguacguggaccaggaacuggacaucaaccggcuguccgacuacgauguggaccacaucgugccucagagcuuucugaaggacgacuccaucgacaacaaggugcugaccagaagcgacaaggcccggggcaagagcgacaacgugcccuccgaagaggucgugaagaagaugaagaacuacuggcggcagcugcugaacgccaagcugauuacccagagaaaguucgacaaucugaccaaggccgagagaggcggccugagcgaacuggauaaggccggcuucaucaagagacagcugguggaaacccggcagaucacaaagcacguggcacagauccuggacucccggaugaacacuaaguacgacgagaaugacaagcugauccgggaagugaaagugaucacccugaaguccaagcugguguccgauuuccggaaggauuuccaguuuuacaaagugcgcgagaucaacaacuaccaccacgcccacgacgccuaccugaacgccgucgugggaaccgcccugaucaaaaaguacccuaagcuggaaagcgaguucguguacggcgacuacaagguguacgacgugcggaagaugaucgccaagagcgagcaggaaaucggcaaggcuaccgccaaguacuucuucuacagcaacaucaugaacuuuuucaagaccgagauuacccuggccaacggcgagauccggaagcggccucugaucgagacaaacggcgaaaccggggagaucgugugggauaagggccgggauuuugccaccgugcggaaagugcugagcaugccccaagugaauaucgugaaaaagaccgaggugcagacaggcggcuucagcaaagagucuauccugcccaagaggaacagcgauaagcugaucgccagaaagaaggacugggacccuaagaaguacggcggcuucgacagccccaccguggccuauucugugcuggugguggccaaaguggaaaagggcaaguccaagaaacugaagagugugaaagagcugcuggggaucaccaucauggaaagaagcagcuucgagaagaaucccaucgacuuucuggaagccaagggcuacaaagaagugaaaaaggaccugaucaucaagcugccuaaguacucccuguucgagcuggaaaacggccggaagagaaugcuggccucugccggcgaacugcagaagggaaacgaacuggcccugcccuccaaauaugugaacuuccuguaccuggccagccacuaugagaagcugaagggcucccccgaggauaaugagcagaaacagcuguuuguggaacagcacaagcacuaccuggacgagaucaucgagcagaucagcgaguucuccaagagagugauccuggccgacgcuaaucuggacaaagugcuguccgccuacaacaagcaccgggauaagcccaucagagagcaggccgagaauaucauccaccuguuuacccugaccaaucugggagccccugccgccuucaaguacuuugacaccaccaucgaccggaagagguacaccagcaccaaagaggugcuggacgccacccugauccaccagagcaucaccggccuguacgagacacggaucgaccugucucagcugggaggcgacagcgcuggaggagguggaagcggaggaggaggaagcggaggaggagguagcggaccuaagaaaaagaggaagguggcggccgcuggauccggacgggcugacgcauuggacgauuuugaucuggauaugcugggaagugacgcccucgaugauuuugaccuugacaugcuugguucggaugcccuugaugacuuugaccucgacaugcucggcagugacgcccuugaugauuucgaccuggacaugcugauuaacuguacaggcaguggagagggcagaggaagucugcuaacaugcggugacgucgaggagaauccuggcccaauggcuucaaacuuuacucaguucgugcucguggacaauggugggacaggggaugugacaguggcuccuucuaauuucgcuaaugggguggcagaguggaucagcuccaacucacggagccaggccuacaaggugacaugcagcgucaggcagucuagugcccagaagagaaaguauaccaucaagguggagguccccaaaguggcuacccagacagugggcggagucgaacugccugucgccgcuuggagguccuaccugaacauggagcucacuaucccaauuuucgcuaccaauucugacugugaacucaucgugaaggcaaugcaggggcuccucaaagacgguaauccuaucccuuccgccaucgccgcuaacucagguaucuacagcgcuggaggagguggaagcggaggaggaggaagcggaggaggagguagcggaccuaagaaaaagaggaagguggcggccgcuggauccccuucagggcagaucagcaaccaggcccuggcucuggccccuagcuccgcuccagugcuggcccagacuauggugcccucuagugcuauggugccucuggcccagccaccugcuccagccccugugcugaccccaggaccaccccagucacugagcgcuccagugcccaagucuacacaggccggcgaggggacucugagugaagcucugcugcaccugcaguucgacgcugaugaggaccugggagcucugcuggggaacagcaccgaucccggaguguucacagaucuggccuccguggacaacucugaguuucagcagcugcugaaucagggcguguccaugucucauaguacagccgaaccaaugcugauggaguaccccgaagccauuacccggcuggugaccggcagccagcggccccccgaccccgcuccaacuccccugggaaccagcggccugccuaaugggcuguccggagaugaagacuucucaagcaucgcugauauggacuuuagugcccugcugucacagauuuccucuagugggcagggaggagguggaagcggcuucagcguggacaccagugcccugcuggaccuguucagccccucggugaccgugcccgacaugagccugccugaccuugacagcagccuggccaguauccaagagcuccugucuccccaggagccccccaggccucccgaggcagagaacagcagcccggauucagggaagcagcuggugcacuacacagcgcagccgcuguuccugcuggaccccggcuccguggacaccgggagcaacgaccugccggugcuguuugagcugggagagggcuccuacuucuccgaaggggacggcuucgccgaggaccccaccaucucccugcugacaggcucggagccucccaaagccaaggaccccacugucucc

(19) United States
(12) Patent Application Publication
(10) Pub. No.: US 2021/0079394 A1
(43) Pub. Date: Mar. 18, 2021

(54) TRANSCRIPTION MODULATION IN ANIMALS USING CRISPR/CAS SYSTEMS DELIVERED BY LIPID NANOPARTICLES

(71) Applicant: Regeneron Pharmaceuticals, Inc., Tarrytown, NY (US)

(72) Inventors: CHARLEEN HUNT, Dumont, NJ (US); Suzanne Hartford, Putnam Valley, NY (US); Guochun Gong, Pleasantville, NY (US); Brian Zambrowicz, Sleepy Hollow, NY (US)

(21) Appl. No.: 17/018,680

(22) Filed: Sep. 11, 2020

Related U.S. Application Data
(60) Provisional application No. 62/900,080, filed on Sep. 13, 2019.
(60) Provisional application No. 63/042,762, filed on Jun. 23, 2020.

(51) Int. Cl. C12N 15/113; C12N 9/22; A61K 9/00; A61K 47/69

(52) U.S. Cl. CPC C12N 15/113; C12N 9/22; A61K 9/0019; C12N 2310/321; C12N 2310/20; C12N 2310/313; A61K 47/6929

(57) ABSTRACT

Lipid nanoparticles comprising CRISPR/Cas synergistic activation mediator system components together in the same lipid nanoparticle and methods of using such lipid nanoparticles to increase expression of target genes in vivo and ex vivo and to assess CRISPR/Cas synergistic activation mediator systems for the ability to increase expression of target genes in vivo and ex vivo are provided.

### CROSS REFERENCE TO RELATED APPLICATIONS

This application claims the benefit of U.S. Application No. 62/900,080, filed Sep. 13, 2019, and U.S. Application No. 63/042,762, filed Jun. 23, 2020, each of which is herein incorporated by reference in its entirety for all purposes.

### REFERENCE TO A SEQUENCE LISTING SUBMITTED AS A TEXT FILE VIA EFS WEB

The Sequence Listing written in file 693474SEQLIST.txt is 122 kilobytes, was created on Sep. 11, 2020, and is hereby incorporated by reference.

### BACKGROUND

Gene expression in strictly controlled in many biological processes, such as development and diseases. Transcription factors regulate gene expression by binding to specific DNA sequences at the enhancer and promoter regions of target genes and modulate transcription through their effector domains. Based on the same principle, artificial transcription factors (ATFs) have been generated by fusing various functional domains to a DNA binding domain engineered to bind to genes of interest, thereby modulating their expression. However, binding specificity of these ATFs is usually degenerate and can be difficult to predict, and the complex and time-consuming design and generation of ATFs limits their applications.

CRISPR/Cas-based activation is a powerful tool for functional gene interrogation, but delivery difficulties have limited its applications in vivo. One limitation in vivo is the need to simultaneously introduce all components into a living organism such that all of the components reach the same cells and induce a robust and sustained increase in transcription of target genes. Better methods and tools are needed to introduce CRISPR/Cas agents in vivo.

### SUMMARY

Lipid nanoparticles comprising CRISPR/Cas synergistic activation mediator system components together in the same lipid nanoparticle and methods of using such lipid nanoparticles to increase expression of target genes in vivo and ex vivo in eukaryotic genomes, cells, and organisms and to assess CRISPR/Cas synergistic activation mediator systems for the ability to increase expression of target genes in vivo and ex vivo in eukaryotic genomes, cells, and organisms are provided.

In one aspect, provided are lipid nanoparticles (LNPs) for delivering a cargo to a target gene to increase expression of the target gene in an animal or cell. In some such LNPs, the cargo comprises: (a) a nucleic acid encoding a chimeric Clustered Regularly Interspaced Short Palindromic Repeats (CRISPR) associated (Cas) protein comprising a nuclease-inactive Cas protein fused to one or more transcriptional activation domains; (b) a nucleic acid encoding a chimeric adaptor protein comprising an adaptor protein fused to one or more transcriptional activation domains; and (c) one or more guide RNAs or one or more nucleic acids encoding the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within the target gene, thereby increasing expression of the target gene.

In some such LNPs, a multicistronic or bicistronic nucleic acid comprises (a) and (b). Optionally, (a) and (b) are linked by a 2A protein coding sequence in the multicistronic or bicistronic nucleic acid. In some such LNPs, (a) and (b) are separate nucleic acids. In some such LNPs, (a) and (b) are each in the form of a messenger RNA (mRNA). Optionally, the mRNA is modified to be fully substituted with pseudouridine. Optionally, the mRNA is a multicistronic or bicistronic nucleic acid comprising (a) and (b), wherein the mRNA comprises the sequence set forth in SEQ ID NO: 61 or comprises a sequence at least 90%, 95%, 96%, 97%, 98%, 99%, or 100% identical to the sequence set forth in SEQ ID NO: 61 (and optionally encodes the same protein as SEQ ID NO: 61). In some such LNPs, (c) is in the form of RNA. Optionally, each of the one or more guide RNAs is modified to comprise one or more stabilizing end modifications at the 5′ end and/or the 3′ end. Optionally, the 5′ end and/or the 3′ end of each of the one or more guide RNAs is modified to comprise one or more phosphorothioate linkages. Optionally, the 5′ end and/or the 3′ end of each of the one or more guide RNAs is modified to comprise one or more 2′-O-methyl modifications.

In some such LNPs, the target sequence comprises a regulatory sequence within the target gene. Optionally, the regulatory sequence comprises a promoter or an enhancer. In some such LNPs, the target sequence is within 200 base pairs of the transcription start site of the target gene. Optionally, the target sequence is within the region 200 base pairs upstream of the transcription start site and 1 base pair downstream of the transcription start site.

In some such LNPs, each of the one or guide RNAs comprises two adaptor-binding elements to which the chimeric adaptor protein can specifically bind. Optionally, a first adaptor-binding element is within a first loop of each of the one or more guide RNAs, and a second adaptor-binding element is within a second loop of each of the one or more guide RNAs. Optionally, each of the one or more guide RNAs is a single guide RNA comprising a CRISPR RNA (crRNA) portion fused to a transactivating CRISPR RNA (tracrRNA) portion, and the first loop is the tetraloop corresponding to residues 13-16 of SEQ ID NO: 12, 14, 52, or 53, and the second loop is the stem loop 2 corresponding to residues 53-56 of SEQ ID NO: 12, 14, 52, or 53.

In some such LNPs, the adaptor-binding element comprises the sequence set forth in SEQ ID NO: 16. Optionally, each of the one or more guide RNAs comprises the sequence set forth in SEQ ID NO: 40, 45, 56, or 57.

In some such LNPs, at least one of the one or more guide RNAs targets a Ttr gene, optionally wherein the Ttr-targeting guide RNA targets a sequence comprising the sequence set forth in any one of SEQ ID NOS: 34-36 or optionally wherein the Ttr-targeting guide RNA comprises the sequence set forth in any one of SEQ ID NOS: 37-39 and 55.

In some such LNPs, the one or more guide RNAs target two or more target genes. In some such LNPs, the one or more guide RNAs comprise multiple guide RNAs that target a single target gene. In some such LNPs, the one or more guide RNAs comprise at least three guide RNAs that target a single target gene. Optionally, the at least three guide RNAs target the mouse Ttr locus, and wherein a first guide RNA targets a sequence comprising SEQ ID NO: 34 or comprises the sequence set forth in SEQ ID NO: 37, a second guide RNA targets a sequence comprising SEQ ID NO: 35 or comprises the sequence set forth in SEQ ID NO: 38, and a third guide RNA targets a sequence comprising SEQ ID NO: 36 or comprises the sequence set forth in SEQ ID NO: 39 or 55.

In some such LNPs, the Cas protein is a Cas9 protein. Optionally, the Cas9 protein is a Streptococcus pyogenes Cas9 protein, a Campylobacter jejuni Cas9 protein, or a Staphylococcus aureus Cas9 protein. Optionally, the Cas9 protein comprises mutations corresponding to D10A and N863A or D10A and H840A when optimally aligned with a Streptococcus pyogenes Cas9 protein.

In some such LNPs, the sequence encoding the Cas protein is codon-optimized for expression in the animal or cell.

In some such LNPs, the one or more transcriptional activator domains in the chimeric Cas protein are selected from: VP16, VP64, p65, MyoD1, HSF1, RTA, SET7/9, and a combination thereof. Optionally, the one or more transcriptional activator domains in the chimeric Cas protein comprise VP64. Optionally, the chimeric Cas protein comprises from N-terminus to C-terminus: the catalytically inactive Cas protein; a nuclear localization signal; and the VP64 transcriptional activator domain. Optionally, the chimeric Cas protein comprises a sequence at least 90%, 95%, 96%, 97%, 98%, 99%, or 100% identical to the sequence set forth in SEQ ID NO: 1. Optionally, the nucleic acid encoding the chimeric Cas protein comprises a sequence at least 90%, 95%, 96%, 97%, 98%, 99%, or 100% identical to the sequence set forth in SEQ ID NO: 25.

In some such LNPs, the adaptor protein is at the N-terminal end of the chimeric adaptor protein, and the one or more transcriptional activation domains are at the C-terminal end of the chimeric adaptor protein. In some such LNPs, the adaptor protein comprises an MS2 coat protein or a functional fragment or variant thereof. In some such LNPs, the one or more transcriptional activation domains in the chimeric adaptor protein are selected from: VP16, VP64, p65, MyoD1, HSF1, RTA, SET7/9, and a combination thereof. Optionally, the one or more transcriptional activation domains in the chimeric adaptor protein comprise p65 and HSF1. Optionally, the chimeric adaptor protein comprises from N-terminus to C-terminus: an MS2 coat protein; a nuclear localization signal; the p65 transcriptional activation domain; and the HSF1 transcriptional activation domain. Optionally, the chimeric adaptor protein comprises a sequence at least 90%, 95%, 96%, 97%, 98%, 99%, or 100% identical to the sequence set forth in SEQ ID NO: 6. Optionally, the nucleic acid encoding the chimeric adaptor protein comprises a sequence at least 90%, 95%, 96%, 97%, 98%, 99%, or 100% identical to the sequence set forth in SEQ ID NO: 27.

In some such LNPs, the animal is a non-human animal. In some such LNPs, the animal is a mammal. Optionally, the mammal is a rodent. Optionally, the rodent is a rat or a mouse. Optionally, the rodent is the mouse. In some such LNPs, the animal is a human. In some such LNPs, the target gene is a gene expressed in the liver.

In some such LNPs, the target gene is a disease-associated gene. In some such LNPs, decreased expression or activity of the target gene is associated with or causative of a disease, disorder, or syndrome. In some such LNPs, the target gene is a haploinsufficient gene or is OTC, HBG1, or HBG2. Optionally, the haploinsufficient gene is KCNQ4, PINK1, TP73, GLUT1, MYH, ABCA4, LRH-1, PAX8, SLC40A1, BMPR2, PKD2, PIK3R1, HMGA1, GCK, ELN, GTF3, GATA3, BUB3, PAX6, FLI1, HNF1A, PKD1, MC4R, DMPK, or MYH9. Optionally, the haploinsufficient gene is any one of the genes in Table 2 or Table 3. In some such LNPs, increased expression or activity of the target gene is associated with or causative of a disease, disorder, or syndrome.

Some such LNPs comprise a cationic lipid, a neutral lipid, a helper lipid, and a stealth lipid. Optionally, the cationic lipid is MC3 and/or the neutral lipid is DSPC and/or the helper lipid is cholesterol and/or the stealth lipid is PEG-DMG. Optionally, the LNP comprises MC3, DSPC, cholesterol, and PEG-DMG in a molar ratio of about 50:10:38.5:1.5.

In another aspect, provided are methods for increasing expression of a target gene in an animal in vivo or an animal cell ex vivo or in vivo. Likewise, provided are methods for increasing expression of a target gene in an animal cell in vitro. Some such methods comprise introducing into the animal or cell: (a) a nucleic acid encoding a chimeric Clustered Regularly Interspaced Short Palindromic Repeats (CRISPR) associated (Cas) protein comprising a nuclease-inactive Cas protein fused to one or more transcriptional activation domains; (b) a nucleic acid encoding a chimeric adaptor protein comprising an adaptor protein fused to one or more transcriptional activation domains; and (c) one or more guide RNAs or one or more nucleic acids encoding the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within the target gene, thereby increasing expression of the target gene, wherein (a), (b), and (c) are delivered together in the same lipid nanoparticle (LNP).

In some such methods, a multicistronic or bicistronic nucleic acid comprises (a) and (b). Optionally, (a) and (b) are linked by a 2A protein coding sequence in the multicistronic or bicistronic nucleic acid. In some such methods, (a) and (b) are separate nucleic acids. In some such methods, (a) and (b) are each introduced in the form of a messenger RNA (mRNA). Optionally, the mRNA is modified to be fully substituted with pseudouridine. Optionally, the mRNA is a multicistronic or bicistronic nucleic acid comprising (a) and (b), wherein the mRNA comprises the sequence set forth in SEQ ID NO: 61 or comprises a sequence at least 90%, 95%, 96%, 97%, 98%, 99%, or 100% identical to the sequence set forth in SEQ ID NO: 61 (and optionally encodes the same protein as SEQ ID NO: 61). In some such methods, (c) is introduced in the form of RNA. Optionally, each of the one or more guide RNAs is modified to comprise one or more stabilizing end modifications at the 5′ end and/or the 3′ end. Optionally, the 5′ end and/or the 3′ end of each of the one or more guide RNAs is modified to comprise one or more phosphorothioate linkages. Optionally, the 5′ end and/or the 3′ end of each of the one or more guide RNAs is modified to comprise one or more 2′-O-methyl modifications.

In some such methods, the target sequence comprises a regulatory sequence within the target gene. Optionally, the regulatory sequence comprises a promoter or an enhancer. In some such methods, the target sequence is within 200 base pairs of the transcription start site of the target gene. Optionally, the target sequence is within the region 200 base pairs upstream of the transcription start site and 1 base pair downstream of the transcription start site.

In some such methods, each of the one or guide RNAs comprises two adaptor-binding elements to which the chimeric adaptor protein can specifically bind. Optionally, a first adaptor-binding element is within a first loop of each of the one or more guide RNAs, and a second adaptor-binding element is within a second loop of each of the one or more guide RNAs. Optionally, each of the one or more guide RNAs is a single guide RNA comprising a CRISPR RNA (crRNA) portion fused to a transactivating CRISPR RNA (tracrRNA) portion, and the first loop is the tetraloop corresponding to residues 13-16 of SEQ ID NO: 12, 14, 52, or 53, and the second loop is the stem loop 2 corresponding to residues 53-56 of SEQ ID NO: 12, 14, 52, or 53.

In some such methods, the adaptor-binding element comprises the sequence set forth in SEQ ID NO: 16. Optionally, each of the one or more guide RNAs comprises the sequence set forth in SEQ ID NO: 40, 45, 56, or 57.

In some such methods, at least one of the one or more guide RNAs targets a Ttr gene, optionally wherein the Ttr-targeting guide RNA targets a sequence comprising the sequence set forth in any one of SEQ ID NOS: 34-36 or optionally wherein the Ttr-targeting guide RNA comprises the sequence set forth in any one of SEQ ID NOS: 37-39 and 55.

In some such methods, the one or more guide RNAs target two or more target genes. In some such methods, the one or more guide RNAs comprise multiple guide RNAs that target a single target gene. In some such methods, the one or more guide RNAs comprise at least three guide RNAs that target a single target gene. Optionally, the at least three guide RNAs target the mouse Ttr locus, and wherein a first guide RNA targets a sequence comprising SEQ ID NO: 34 or comprises the sequence set forth in SEQ ID NO: 37, a second guide RNA targets a sequence comprising SEQ ID NO: 35 or comprises the sequence set forth in SEQ ID NO: 38, and a third guide RNA targets a sequence comprising SEQ ID NO: 36 or comprises the sequence set forth in SEQ ID NO: 39 or 55.

In some such methods, the Cas protein is a Cas9 protein. Optionally, the Cas9 protein is a Streptococcus pyogenes Cas9 protein, a Campylobacter jejuni Cas9 protein, or a Staphylococcus aureus Cas9 protein. Optionally, the Cas9 protein comprises mutations corresponding to D10A and N863A or D10A and H840A when optimally aligned with a Streptococcus pyogenes Cas9 protein.

In some such methods, the sequence encoding the Cas protein is codon-optimized for expression in the animal.

In some such methods, the one or more transcriptional activator domains in the chimeric Cas protein are selected from: VP16, VP64, p65, MyoD1, HSF1, RTA, SET7/9, and a combination thereof. Optionally, the one or more transcriptional activator domains in the chimeric Cas protein comprise VP64. Optionally, the chimeric Cas protein comprises from N-terminus to C-terminus: the catalytically inactive Cas protein; a nuclear localization signal; and the VP64 transcriptional activator domain. Optionally, the chimeric Cas protein comprises a sequence at least 90%, 95%, 96%, 97%, 98%, 99%, or 100% identical to the sequence set forth in SEQ ID NO: 1. Optionally, the nucleic acid encoding the chimeric Cas protein comprises a sequence at least 90%, 95%, 96%, 97%, 98%, 99%, or 100% identical to the sequence set forth in SEQ ID NO: 25.

In some such methods, the adaptor protein is at the N-terminal end of the chimeric adaptor protein, and the one or more transcriptional activation domains are at the C-terminal end of the chimeric adaptor protein. In some such methods, the adaptor protein comprises an MS2 coat protein or a functional fragment or variant thereof. In some such methods, the one or more transcriptional activation domains in the chimeric adaptor protein are selected from: VP16, VP64, p65, MyoD1, HSF1, RTA, SET7/9, and a combination thereof. Optionally, the one or more transcriptional activation domains in the chimeric adaptor protein comprise p65 and HSF1. Optionally, the chimeric adaptor protein comprises from N-terminus to C-terminus: an MS2 coat protein; a nuclear localization signal; the p65 transcriptional activation domain; and the HSF1 transcriptional activation domain. Optionally, the chimeric adaptor protein comprises a sequence at least 90%, 95%, 96%, 97%, 98%, 99%, or 100% identical to the sequence set forth in SEQ ID NO: 6. Optionally, the nucleic acid encoding the chimeric adaptor protein comprises a sequence at least 90%, 95%, 96%, 97%, 98%, 99%, or 100% identical to the sequence set forth in SEQ ID NO: 27.

In some such methods, the animal is a non-human animal. In some such methods, the animal is a mammal. Optionally, the mammal is a rodent. Optionally, the rodent is a rat or a mouse. Optionally, the rodent is the mouse. In some such methods, the animal is a human. In some such methods, the animal is a subject in need of increased expression of the target gene, wherein the target gene is underexpressed in the subject, and the underexpression is associated with or causative of a disease, disorder, or syndrome in the subject. In some such methods, the target gene is a gene expressed in the liver. In some such methods, the route of administration of the one or more guide RNAs to the animal is intravenous injection, intraparenchymal injection, intraperitoneal injection, nasal installation, or intravitreal injection.

In some such methods, the target gene is a disease-associated gene. In some such methods, decreased expression or activity of the target gene is associated with or causative of a disease, disorder, or syndrome. In some such methods, the target gene is a haploinsufficient gene or is OTC, HBG1, or HBG2. Optionally, the haploinsufficient gene is KCNQ4, PINK1, TP73, GLUT1, MYH, ABCA4, LRH-1, PAX8, SLC40A1, BMPR2, PKD2, PIK3R1, HMGA1, GCK, ELN, GTF3, GATA3, BUB3, PAX6, FLI1, HNF1A, PKD1, MC4R, DMPK, or MYH9. Optionally, the haploinsufficient gene is any one of the genes in Table 2 or Table 3. In some such methods, increased expression or activity of the target gene is associated with or causative of a disease, disorder, or syndrome.

In some such methods, the lipid nanoparticle comprises a cationic lipid, a neutral lipid, a helper lipid, and a stealth lipid. Optionally, the cationic lipid is MC3 and/or the neutral lipid is DSPC and/or the helper lipid is cholesterol and/or the stealth lipid is PEG-DMG. Optionally, the lipid nanoparticle comprises MC3, DSPC, cholesterol, and PEG-DMG in a molar ratio of about 50:10:38.5:1.5.

In some such methods, the increase in expression of the target gene is at least 0.5-fold, 1-fold, 2-fold, 3-fold, 4-fold, 5-fold, 6-fold, 7-fold, 8-fold, 9-fold, 10-fold, or 20-fold higher relative to a control animal or cell. In some such methods, the duration of the increase in expression of the target gene is at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, or at least about 2 months.

In some such methods, the lipid nanoparticle comprising (a), (b), and (c) is introduced into the animal or cell two or more times sequentially. In some such methods, the lipid nanoparticle comprising (a), (b), and (c) is introduced into the animal or cell three or more times sequentially. Optionally, expression of the target gene is increased to at least the same level after each sequential introduction of the lipid nanoparticle. Optionally, expression of the target gene is increased to a higher level than in methods in which the lipid nanoparticle is introduced only once.

### BRIEF DESCRIPTION OF THE FIGURES

FIG. 1 (not to scale) shows a schematic for a Ttr guide RNA array. The guide RNA array allele comprises from 5′ to 3′: a first U6 promoter; a first guide RNA coding sequence; a second U6 promoter; a second guide RNA coding sequence; a third U6 promoter; and a third guide RNA coding sequence.

FIG. 2 (not to scale) shows a schematic for designing three guide RNAs that target upstream of the transcription start site of Ttr.

FIG. 3 shows a schematic of a generic single guide RNA (SEQ ID NO: 45) in which the tetraloop and stem loop 2 have been replaced with MS2-binding aptamers to facilitate recruitment of chimeric MS2 coat protein (MCP) fused to transcriptional activation domains.

FIG. 4 shows circulating serum levels of TTR in untreated dCas9 SAM mice, dCas9 SAM mice treated with AAV8-GFP, and dCas9 SAM mice treated with AAV8 comprising a Ttr guide RNA array as assayed by ELISA. Results from 5 days, 19 days, 2 months, 3 months, 4 months, 5 months, 6 months, 7 months, and 8 months post-injection are shown.

FIGS. 5A and 5B show circulating serum levels of TTR (FIG. 5A) and percent change in circulating serum levels of TTR from baseline (FIG. 5B) in untreated dCas9 SAM mice and dCas9 SAM mice treated with LNP comprising a Ttr guide RNA (R-LNP 277) as assayed by ELISA. Results from 1, 3, 6, 8, 10, 13, 17, 20, 27, 34, and 67 days post-injection are shown.

FIG. 6 shows circulating levels of TTR in untreated dCas9 SAM mice and dCas9 SAM mice treated with LNP comprising a Ttr guide RNA (R-LNP 277) as assayed by ELISA. Results from doses of 0.5 mpk, 1 mpk, and 2 mpk are shown at 1 week, 2 weeks, 3 weeks, 4 weeks, 5 weeks, 6 weeks, and 7 weeks post-injection. All values are plotted as mean+/−SD. Asterisks indicate significance, and the number of asterisks indicates the number of Os after the decimal point (TTEST).

FIG. 7A shows circulating levels of TTR following sequential dosing of LNP^{TtrgA2} at four weeks in dCas9 SAM mice (R26^{SAM/SAM}). LNP particles were formulated with 0.5 mpk of synthetic Ttr gA2 SAM guides and introduced into homozygous dCas9 SAM mice (R26^{SAM/SAM}) (n=5) at zero weeks and/or four weeks. Protein expression levels were determined by ELISA with weekly bleeds. All values are plotted as mean+/−SD. Asterisks indicate significance, and the number of asterisks indicates the number of Os after the decimal point (TTEST).

FIG. 7B shows circulating levels of TTR following sequential dosing of LNP^{TtrgA2} at two weeks in dCas9 SAM mice (R26^{SAM/SAM}). LNP particles were formulated with 0.5 mpk of synthetic Ttr gA2 SAM guides and introduced into homozygous dCas9 SAM mice (R26^{SAM/SAM}) (n=5) at zero weeks and/or two weeks. Protein expression levels were determined by ELISA with weekly bleeds. All values are plotted as mean+/−SD. Asterisks indicate significance, and the number of asterisks indicates the number of Os after the decimal point (TTEST).

FIG. 8 shows circulating levels of TTR following sequential dosing of LNP^{TtrgA2 at} zero weeks, two weeks, and four weeks in dCas9 SAM mice (R26^{SAM/SAM}). LNP particles were formulated with 0.5 mpk of synthetic Ttr gA2 SAM guides and introduced into homozygous dCas9 SAM mice (R26^{SAM/SAM}) (n=5) at zero weeks, two weeks, and four weeks or only at zero weeks. Protein expression levels were determined by ELISA with weekly bleeds. All values are plotted as mean+/−SD.

FIG. 9 shows circulating levels of TTR following dosing of wild type mice with LNP particles formulated with synthetic Ttr SAM guides and SAM mRNA (either pseudouridine-modified or unmodified). Untreated mice were used as a negative control. Protein expression levels were determined by ELISA with bleeds at the indicated time points. All values are plotted as mean+/−SEM.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “targeting vector” refers to a recombinant nucleic acid that can be introduced by homologous recombination, non-homologous-end-joining-mediated ligation, or any other means of recombination to a target position in the genome of a cell.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” also includes proteins and nucleic acids that have no naturally occurring counterpart or proteins or nucleic acids that have been chemically synthesized and are thus substantially uncontaminated by other proteins or nucleic acids. The term “isolated” also includes proteins, nucleic acids, or cells that have been separated or purified from most other cellular components or organism components with which they are naturally accompanied (e.g., other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or eukaryotic organism (e.g., animal, non-human animal, mammal, or non-human mammal). For example, an endogenous Ttr sequence of a non-human animal refers to a native Ttr sequence that naturally occurs at the Ttr locus in the non-human animal.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “Ttr locus” may refer to the specific location of a Ttr gene, Ttr DNA sequence, TTR-encoding sequence, or Ttr position on a chromosome of the genome of an organism that has been identified as to where such a sequence resides. A “Ttr locus” may comprise a regulatory element of a Ttr gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Complementarity” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations consider nucleic acid structural characteristics.

“Hybridization condition” includes the cumulative environment in which one nucleic acid strand bonds to a second nucleic acid strand by complementary strand interactions and hydrogen bonding to produce a hybridization complex. Such conditions include the chemical components and their concentrations (e.g., salts, chelating agents, formamide) of an aqueous or organic solution containing the nucleic acids, and the temperature of the mixture. Other factors, such as the length of incubation time or reaction chamber dimensions may contribute to the environment. See, e.g., Sambrook et al., Molecular Cloning, A Laboratory Manual, 2.sup.nd ed., pp. 1.90-1.91, 9.47-9.51, 1 1.47-11.57 (Cold Spring Harbor Laboratory Press, Cold Spring Harbor, N.Y., 1989), herein incorporated by reference in its entirety for all purposes.

Hybridization requires that the two nucleic acids contain complementary sequences, although mismatches between bases are possible. The conditions appropriate for hybridization between two nucleic acids depend on the length of the nucleic acids and the degree of complementation, variables which are well known. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer, 30 or fewer, 25 or fewer, 22 or fewer, 20 or fewer, or 18 or fewer nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid include at least about 15 nucleotides, at least about 20 nucleotides, at least about 22 nucleotides, at least about 25 nucleotides, and at least about 30 nucleotides. Furthermore, the temperature and wash solution salt concentration may be adjusted as necessary according to factors such as length of the region of complementation and the degree of complementation.

The sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide (e.g., gRNA) can comprise at least 70%, at least 80%, at least 90%, at least 95%, at least 99%, or 100% sequence complementarity to a target region within the target nucleic acid sequence to which they are targeted. For example, a gRNA in which 18 of 20 nucleotides are complementary to a target region, and would therefore specifically hybridize, would represent 90% complementarity. In this example, the remaining noncomplementary nucleotides may be clustered or interspersed with complementary nucleotides and need not be contiguous to each other or to complementary nucleotides.

Percent complementarity between particular stretches of nucleic acid sequences within nucleic acids can be determined routinely using BLAST programs (basic local alignment search tools) and PowerBLAST programs (Altschul et al. (1990) J. Mol. Biol. 215(3):403-410; Zhang and Madden (1997) Genome Res. 7(6):649-656) or by using the Gap program (Wisconsin Sequence Analysis Package, Version 8 for Unix, Genetics Computer Group, University Research Park, Madison Wis.), using default settings, which uses the algorithm of Smith and Waterman (1981) Adv. Appl. Math. 2(4):482-489.

The methods and compositions provided herein employ a variety of different components. Some components throughout the description can have active variants and fragments. Such components include, for example, Cas proteins, CRISPR RNAs, tracrRNAs, and guide RNAs. Biological activity for each of these components is described elsewhere herein. The term “functional” refers to the innate ability of a protein or nucleic acid (or a fragment or variant thereof) to exhibit a biological activity or function. Such biological activities or functions can include, for example, the ability of a Cas protein to bind to a guide RNA and to a target DNA sequence. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized in Table 1 below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or in isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “reporter gene” refers to a nucleic acid having a sequence encoding a gene product (typically an enzyme) that is easily and quantifiably assayed when a construct comprising the reporter gene sequence operably linked to an endogenous or heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreen1), yellow fluorescent proteins (e.g., YFP, eYFP, Citrine, Venus, YPet, PhiYFP, and ZsYellow1), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values within a standard margin of error of measurement (e.g., SEM) of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Unless otherwise indicated, statistically significant means p<0.05.

### DETAILED DESCRIPTION

### I. Overview

Lipid nanoparticles comprising CRISPR/Cas synergistic activation mediator system components together in the same lipid nanoparticle and methods of using such lipid nanoparticles to increase expression of target genes in vivo and ex vivo and to assess CRISPR/Cas synergistic activation mediator systems for the ability to increase expression of target genes in vivo and ex vivo are provided.

CRISPR/Cas9, an RNA-guided DNA endonuclease, catalyzes the formation of double-strand breaks in DNA at the binding site of its guide RNA. Two important catalytic domains have been identified in the Cas9: the RuvC and HNH domains. The RuvC domain initiates cleavage of the DNA strand not complementary to the guide RNA, and the HNH domain cleaves the DNA strand complementary to the guide RNA. Either domain can be inactivated to make Cas9 a nickase, or both domains can be mutated to form a catalytically dead Cas9 (dCas9). Though dCas9 cannot cause strand breakage, the catalytically dead protein can be used to shuttle other proteins to specific genomic regions. This is the basis for activation and repression variants of the CRISPR/Cas9 system.

In the dCas9 synergistic activation mediator (SAM) system, several activation domains interact to cause a greater gene response than could be induced by any one factor alone. In the initial iteration of this system, three lentiviruses needed to be introduced. The first lentivirus would contain dCas9 directly fused to a VP64 domain, a transcriptional activator composed of four tandem copies of Herpes Simplex Viral Protein 16. When VP64 is fused to a protein that binds near a transcriptional start site, it acts as a strong transcriptional activator. The second lentivirus would bring in MS2 coat protein (MCP) fused to two additional activating transcription factors: heat-shock factor 1 (HSF1) and transcription factor 65 (p65). The MCP naturally binds to MS2 stem loops. In this system, MCP interacts with MS2 stem loops engineered into the CRISPR associated sgRNA and thereby shuttles the bound transcription factors to the appropriate genomic location. The third lentivirus would introduce the MS2-loop-containing sgRNA. While the three-component system allows for some flexibility in cell culture, this set-up is less desirable in an animal model.

Adeno-associated viruses (AAVs) are generally considered safe for gene therapy because they have low immunogenicity and have a highly predictable integration site (AAVS1 on human chromosome 19). However, to increase their safety as gene therapy vectors, the integrative capacity of the WT AAVs has been eliminated such that these vectors remain as episomes in the host cell nucleus. Upon the introduction to a host, the immune response against the AAV is generally restricted to neutralizing antibodies with no clearly defined cytotoxic response. In dividing cells, the AAV DNA is diluted out through cell division, making it necessary to administer more virus for continued therapeutic response. These subsequent exposures may result in rapid neutralization of the virus and, therefore, a decreased host response. To get around this, researchers will use alternative serotypes for sequential infections, though this is hampered by serotype specificity. Another concern in AAV-based therapeutics is the relatively small cloning capacity: 4.6 kb between the two inverted terminal repeats. As the complete coding sequence of dCas9 SAM is ˜5.8 kb (without a promoter), not all SAM components can be expressed from a single AAV.

One way to get around this is to express the elements across two or more AAVs and hope that they both infect the same cell. However, this is less than desirable for a therapeutic solution. With this in mind, we set out to optimize this system such that it can have a clinical translation.

Lipid nanoparticles (LNPs) make an attractive alternative to AAV use as they safely and effectively deliver nucleic acids to cells by leveraging the endogenous endocytosis mechanism to bring the molecules in via LDL receptors. Variation in the formulation can influence the particle's stability and tropism once introduced into an organism. Furthermore, conjugation of various ligands can further increase target specificity of the LNP. One caveat to this delivery method is the transient effect on host cells, as mRNA delivered to cells can in some cases be cleared within 48 hours of cellular intake. However, there is no immune response to LNP delivery, which allows for well-tolerated sequential dosing. Moreover, in the case of catalytically active Cas9, delivery of catalytically active Cas9 and sgRNA will make permanent changes to the target sequence which can be propagated long after the materials have been cleared from the cell. However, transcriptional activation with catalytically inactive Cas9 (catalytically dead Cas9 or dCas9) does not lead to permanent genetic changes. In addition, the application of this delivery system to delivering dCas9 SAM guide RNAs with stabilizing end modifications has been limited by limitations in RNA synthesis technologies. These limitations have precluded the generation of SAM sgRNAs with stabilizing end modifications, as these molecules are greater than the 110-nucleotide platform maximum.

While upregulation of a target gene via delivery of LNP-formulated SAM gRNAs is expected to last for a significantly shorter time, we were surprisingly able to achieve significant transcriptional activation using LNP-mediated delivery that was far less transient than anticipated. LNP delivery of SAM sgRNA together with all of the other SAM components is a significant enhancement to therapeutic dCas9 SAM applications as we can now (1) ensure that the dCas9 SAM transcript and SAM sgRNA land in the same cell, (2) mediate increased tissue specificity with formulations/ligand incorporations, (3) re-dose organisms without fear of immune response, and (4) generate more stable expression levels. Taken together, this combination of nucleic acid delivery has greatly enhanced the potential dCas9 applications in a safe and unexpectedly stable manner.

### II. Methods of Increasing Transcription or Expression of Target Genes and for Assessing Ability of CRISPR/Cas to Increase Transcription of Expression of Target Genes In Vivo or Ex Vivo

Various methods are provided for increasing or activating expression or transcription of a target gene or assessing the ability of a CRISPR/Cas synergistic activation mediator (SAM) system described herein to increase/activate expression or transcription of a target gene in vivo or ex vivo using the lipid nanoparticles (LNPs) described herein. The methods and compositions can be for increasing transcription or expression of target genes in eukaryotic genomes, cells, or organisms. Such LNPs comprise all of the components of a synergistic activation mediator system (one or more guide RNAs or nucleic acid(s) encoding, a chimeric Cas protein or nucleic acid encoding, and a chimeric adaptor protein or nucleic acid encoding) together in the same LNP. For example, such methods can comprise introducing into the cell or eukaryotic organism (e.g., animal, non-human animal, mammal, or non-human mammal): (a) a nucleic acid encoding a chimeric Clustered Regularly Interspaced Short Palindromic Repeats (CRISPR) associated (Cas) protein comprising a nuclease-inactive Cas protein fused to one or more transcriptional activation domains; (b) a nucleic acid encoding a chimeric adaptor protein comprising an adaptor protein fused to one or more transcriptional activation domains; and (c) one or more guide RNAs or one or more nucleic acids encoding the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within the target gene, thereby increasing expression of the target gene, wherein all three components are delivered together in the same LNP. In one example, a multicistronic or bicistronic nucleic acid (e.g., DNA or mRNA) is introduced that encodes both the chimeric Cas protein and the chimeric adaptor protein (referred to herein as a SAM cassette or a SAM mRNA). For example, the sequence encoding the chimeric Cas protein and the sequence encoding the chimeric adaptor protein can be linked by a sequence encoding a 2A protein as described in more detail elsewhere herein. Introducing into a eukaryotic organism refers to any method for delivering the components into the eukaryotic organism such that they gain access to one or more cells and the target gene(s) within those cells. Likewise, introducing into a cell refers to any method for delivering the components into the cell such that they gain access to the target gene(s) within the cell. Suitable chimeric Cas proteins, chimeric adaptor proteins, and guide RNAs are described in more detail elsewhere herein. The one or more guide RNAs can form complexes with the chimeric Cas protein and chimeric adaptor protein and guide them to target sequences within one or more target genes, thereby increasing expression of the one or more target genes. Such methods can further comprise assessing expression or transcription of the one or more target genes.

The various methods provided for increasing or activating expression or transcription of a target gene or assessing the ability of a CRISPR/Cas SAM system to increase/activate expression or transcription of a target gene in vivo can also be used for increasing or activating expression or transcription of a target gene or assessing the ability of a CRISPR/Cas SAM system to increase/activate expression or transcription of a target gene ex vivo in cells. The various methods provided for increasing or activating expression or transcription of a target gene or assessing the ability of a CRISPR/Cas SAM system to increase/activate expression or transcription of a target gene in vivo can also be used for increasing or activating expression or transcription of a target gene or assessing the ability of a CRISPR/Cas SAM system to increase/activate expression or transcription of a target gene in vitro in cells.

In some methods, the cell or organism can be re-dosed with the same lipid nanoparticle two or more times sequentially. For example, the lipid nanoparticle can be introduced into the cell or organism at least about 2, at least about 3, at least about 4, at least about 5, at least about 6, at least about 7, at least about 8, at least about 9, or at least about 10 times sequentially. The interval between doses of the lipid nanoparticle can be any suitable amount of time. For example, the interval can be at least about 1 day, at least about 2 days, at least about 3 days, at least about 4 days, at least about 5 days, at least about 6 days, at least about 7 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, or at least about 4 months. For example, the interval between doses of the lipid nanoparticle can be at least about 1 week (e.g., about 1 week), at least about 2 weeks (e.g., about 2 weeks), at least about 4 weeks (e.g., about 4 weeks), about 1 week to about 5 weeks, about 1 week to about 4 weeks, about 1 week to about 3 weeks, about 1 week to about 2 weeks, about 2 weeks to about 5 weeks, about 2 weeks to about 4 weeks, about 2 weeks to about 3 weeks, about 3 weeks to about 5 weeks, about 3 weeks to about 4 weeks, or about 4 weeks to about 5 weeks. In one example, the interval between doses of the lipid nanoparticle can be about 2 weeks.

In some methods, expression of the target gene is increased to at least about the same level after each sequential introduction of the lipid nanoparticle. In some methods, expression of the target gene is maintained at about the same level by the sequential re-dosing. In some methods, expression of the target gene is increased after re-dosing with the lipid nanoparticle to a higher level than with a single dose of the lipid nanoparticle (e.g., the increase in expression of the target gene with re-dosing of the lipid nanoparticle is higher than the increase in expression of the target gene with no re-dosing).

Optionally, two or more guide RNAs can be introduced, each designed to target a different guide RNA target sequence within a target gene. For example, two or more, three or more, four or more, or five or more guide RNAs can be designed to target a single target gene (e.g., two, three, four, or five guide RNAs can be used, each targeting a different guide RNA target sequence within the same target gene). Alternatively or additionally, two or more, three or more, four or more, or five or more guide RNAs can be introduced, each designed to target different guide RNA target sequences in different target genes (e.g., two or more, three or more, four or more, or five or more different target genes) (i.e., multiplexing). For example, two, three, four, or five guide RNAs can be used, each targeting a different target gene.

Chimeric Cas proteins, chimeric adaptor proteins, and guide RNAs can be introduced into the cell or eukaryotic organism (e.g., animal, non-human animal, mammal, or non-human mammal) in any form (DNA or RNA for guide RNA; DNA, RNA, or protein for chimeric Cas proteins and chimeric adaptor proteins) via any route of administration as disclosed elsewhere herein. The guide RNAs, chimeric Cas proteins, and chimeric adaptor proteins can be introduced in a tissue-specific manner in some methods (e.g., introduced in a liver-specific manner).

Guide RNAs and mRNAs encoding chimeric Cas proteins and chimeric adaptor proteins (e.g., SAM mRNAs) can comprise one or more stabilizing end modifications at the 5′ end and/or 3′ end as described in more detail elsewhere herein. As one example, the 5′ end and/or 3′ end of the RNAs can comprise one or more phosphorothioate linkages. For example, a guide RNA can include phosphorothioate linkages between the 2, 3, or 4 terminal nucleotides at the 5′ or 3′ end of the guide RNA. As another example, the 5′ end and/or 3′ end of the RNAs can comprise one or more 2′-O-methyl modifications. For example, an RNA can include 2′-O-methyl analogs and 3′ phosphorothioate internucleotide linkages at the first three 5′ and 3′ terminal RNA residues. For example, an RNA can include 2′-O-methyl modifications at the 2, 3, or 4 terminal nucleotides at the 5′ and/or 3′ end of the RNA (e.g., the 5′ end). See, e.g., WO 2017/173054 A1 and Finn et al. (2018) Cell Rep. 22(9):2227-2235, each of which is herein incorporated by reference in its entirety for all purposes. As another example, RNAs (e.g., mRNAs) can be capped at the 5′ end (e.g., a cap 1 structure in which the +1 ribonucleotide is methylated at the 2′O position of the ribose, can be polyadenylated, and can optionally also be modified to be fully substituted with pseudouridine (i.e., all standard uracil residues are replaced with pseudouridine, a uridine isomer in which the uracil is attached with a carbon-carbon bond rather than nitrogen-carbon). Other possible modifications to guide RNAs and mRNAs are described in more detail elsewhere herein. In a specific example, an RNA includes 2′-O-methyl analogs and 3′ phosphorothioate internucleotide linkages at the first three 5′ and 3′ terminal RNA residues. Such chemical modifications can, for example, provide greater stability and protection from exonucleases to RNAs, allowing them to persist within cells for longer than unmodified RNAs. Such chemical modifications can also, for example, protect against innate intracellular immune responses that can actively degrade RNA or trigger immune cascades that lead to cell death.

The guide RNAs can target anywhere in the target gene that is suitable for increasing transcription of the target gene. For example, the target sequence for a guide RNA can comprise a regulatory sequence within a target gene, such as a promoter or an enhancer. Likewise, the target sequence can be adjacent to the transcription start site of a gene. For example, the target sequence can be within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair of the transcription start site, within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair upstream of the transcription start site, or within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair downstream of the transcription start site. As a specific example, the target sequence can be within about 200 base pairs of the transcription start site of a target gene or can be within about 200 base pairs upstream of the transcription start site and within 1 base pair downstream of the transcription start site.

The methods disclosed herein can further comprise assessing expression of the target gene. The methods for measuring expression or activity will depend on the target gene being modified. Methods for assessing increased transcription or expression of a target gene are well-known.

For example, if the target gene comprises a gene encoding an RNA or protein, the method of assessing expression can comprise measuring expression or activity of the encoded RNA and/or protein. For example, if the encoded protein is a protein released into the serum, serum levels of the encoded protein can be measured. Assays for measuring levels and activity of RNA and proteins are well-known.

Assessing expression of the target gene in a eukaryotic organism (e.g., animal, non-human animal, mammal, or non-human mammal) can be in any cell type from any tissue or organ. For example, expression of the target gene can be assessed in multiple cell types from the same tissue or organ or in cells from multiple locations within the tissue or organ. This can provide information about which cell types within a target tissue or organ are being targeted or which sections of a tissue or organ are being reached by the CRISPR/Cas and modified. As another example, expression of the target gene can be assessed in multiple types of tissue or in multiple organs. In methods in which a particular tissue or organ is being targeted, this can provide information about how effectively that tissue or organ is being targeted and whether there are off-target effects in other tissues or organs.

In some methods, expression of the target gene in liver cells is assessed, e.g., by assessing serum levels of a secreted protein expressed by the target genomic locus in liver cells. If the target gene encodes a protein with a particular enzymatic activity, assessment can comprise measuring expression of the target gene and/or activity of the protein encoded by the target gene. Alternatively or additionally, assessment can comprise assessing expression in one or more cells isolated from the eukaryotic organism (e.g., animal, non-human animal, mammal, or non-human mammal). Assessment can comprise isolating a target organ or tissue from the eukaryotic organism (e.g., animal, non-human animal, mammal, or non-human mammal) and assessing expression of the target gene in the target organ or tissue. Assessment can also comprise assessing expression of the target gene in two or more different cell types within the target organ or tissue. Similarly, assessment can comprise isolating a non-target organ or tissue (e.g., two or more non-target organs or tissues) from the eukaryotic organism (e.g., animal, non-human animal, mammal, or non-human mammal) and assessing expression of the target gene in the non-target organ or tissue.

In some methods, the target gene can be a disease-associated gene as described elsewhere herein. As one example, the disease-associated gene can be any gene that yields transcription or translation products at an abnormal level or in an abnormal form in cells derived from a disease-affected tissues compared with tissues or cells of a non-disease control. It may be a gene that becomes expressed at an abnormally high level, where the altered expression correlates with the occurrence and/or progression of the disease. It may be a gene that becomes expressed at an abnormally low level, where the altered expression correlates with the occurrence and/or progression of the disease. A disease-associated gene also refers to a gene possessing a mutation or genetic variation that is responsible for the etiology of a disease. The transcribed or translated products may be known or unknown and may be at a normal or abnormal level. For example, the target gene can be a gene associated with a protein aggregation disease or disorder. As a specific example, the target gene can be a gene (e.g., Ttr) associated with a protein aggregation disease or disorder, and the method can comprise increasing expression of that target gene to model the protein aggregation disease or disorder. In some specific methods, the target gene can be Ttr. Optionally, the Ttr gene can comprise a pathogenic mutation (e.g., a mutation causing amyloidosis) or a combination of pathogenic mutations. Examples of such mutations are provided, e.g., in WO 2018/007871, herein incorporated by reference in its entirety for all purposes.

In some methods, the target gene can be any gene (e.g., disease-associated gene) for which increased production of the gene would be beneficial in a subject. For example, reduced transcription of such target genes, reduced amount of the gene products from such target genes, or reduced activity of the gene products from such target genes can be associated with, can exacerbate, or can cause a disease such that increasing transcription or expression of the target gene would be beneficial. One example of such a gene is OTC (Entrez Gene ID 5009). OTC deficiency (ornithine transcarbamylase deficiency) is characterized by elevated ammonia in the blood, which is considered a neurotoxin and can be brought on by a high protein diet. This is an X-linked disease and predominately affects males, but females can develop a milder form due to random X inactivation. There are a number of mutations leading to a range of severity in the disease, as some mutations still allow some wild-type OTC to be made. As one example, a mutant splice site in OTC can result in subjects with about 5% OTC enzymatic activity compared to wild type subjects. It is these patients, along with the symptomatic females, that would benefit from increased expression of OTC through the delivery of SAM mRNA plus a guide RNA targeting the promotor of OTC, as increased expression of wild-type OTC will allow clearance of the excess ammonia in the blood. Other examples of genes for which increased production of the gene would be beneficial in a subject include HBG1 (Entrez Gene ID 3047) and HBG2 (Entrez Gene ID 3048) for increasing fetal hemoglobin expression. Other examples of genes for which increased production of the gene would be beneficial in a subject include haploinsufficient genes. Haploinsufficiency is a situation that occurs when one copy of a gene is inactivated or deleted, and the remaining functional copy of the gene is not adequate to produce the needed gene product to preserve normal function. In other words, for some genes, deletion or inactivation of one functional copy from a diploid genome changes the organism's phenotype to an abnormal or disease state. These genes are called haploinsufficient because one normal copy of these genes is insufficient to produce the normal or wild type phenotype. Loss of one functional copy of haploinsufficient genes has been linked to diseases including neurological disorders and mental retardation, and haploinsufficient genes can also influence a person's susceptibility to disease and/or to the side effects of medications. Examples of haploinsufficient genes and associated diseases/disorders/syndromes associated with loss of one functional copy are provided in Tables 2 and 3. See also Dang et al. (2008) Eur. J. Hum. Genet. 16(11):1350-1357, herein incorporated by reference in its entirety for all purposes.

TABLE 2
Subset of Examples of Haploinsufficient Gene Expression from Table 3.

| Gene | Entrez | |
|---|---|---|
| Symbol | Gene ID | Disorder/Syndrome |
| KCNQ4 | 9132 | deafness, autosomal dominant nonsyndromic |
| | | sensorineural 2 |
| PINK1 | 65018 | sporadic early-onset parkinsonism |
| TP73 | 7161 | prostate hyperplasia and prostate cancer |
| GLUT1 | 6513 | facilitated glucose transporter protein type 1 |
| | | (GLUT1) deficiency syndrome |
| MYH | 4595 | hepatocellular carcinoma and cholangiocarcinoma |
| ABCA4 | 24 | Stargardt disease, retinitis pigmentosa-19, and |
| | | macular degeneration age-related 2 |
| LRH-1 | 2494 | inflammatory bowel disease |
| PAX8 | 7849 | congenital hypothyroidism |
| SLC40A1 | 30061 | ferroportin disease |
| BMPR2 | 659 | primary pulmonary hypertension |
| PKD2 | 5311 | autosomal dominant polycystic kidney disease |
| PIK3R1 | 5295 | insulin resistance |
| HMGA1 | 3159 | insulin resistance and diabetes |
| GCK | 2645 | non-insulin dependent diabetes mellitus (NIDDM), |
| | | maturity onset diabetes of the young, type 2 |
| | | (MODY2) and persistent hyperinsulinemic |
| | | hypoglycemia of infancy (PHHI) |
| ELN | 2006 | cardiovascular disease and connective tissue |
| | | abnormalities |
| GTF3 | 9569 | abnormal muscle fatiguability |
| GATA3 | 2625 | HDR (hypoparathyroidism, deafness and renal |
| | | dysplasia) syndrome |
| BUB3 | 9184 | short life span that is associated with the early |
| | | onset of aging-related features |
| PAX6 | 5080 | eye diseases |
| FLI1 | 2313 | Paris-Trousseau thrombopenia |
| HNF1A | 6927 | reduced serum apolipoprotein M levels |
| PKD1 | 5310 | autosomal dominant polycystic kidney disease |
| MC4R | 4160 | increased adiposity and linear growth |
| DMPK | 1760 | cardiac disease in myotonic dystrophy |
| MYH9 | 4627 | hematological abnormalities |

TABLE 3
Examples of Haploinsufficient Gene Expression.

| | | | Category of |
|---|---|---|---|
| Gene | Entrez | | Disorder/ |
| Symbol | Gene ID | Disorder/Syndrome | Syndrome |
| TP73 | 7161 | prostate hyperplasia and | cancer/ |
| | | prostate cancer | tumorigenesis |
| DFFB | 1677 | oligodendroglioma | cancer/ |
| | | development | tumorigenesis |
| KCNAB2 | 8514 | characteristic craniofacial | mental |
| | | abnormalities, mental | retardation |
| | | retardation, and epilepsy with | |
| | | 1p36 deletion syndrome | |
| CHD5 | 26038 | monosomy 1p36 syndrome | growth and |
| | | | mental |
| | | | retardation |
| CAMTA1 | 23261 | tumors development | cancer/ |
| | | | tumorigenesis |
| PINK1 | 65018 | sporadic early-onset | neurological |
| | | parkinsonism | disorders |
| SAM68 | 10657 | mammary tumor onset and | cancer/ |
| | | tumor multiplicity | tumorigenesis |
| KCNQ4 | 9132 | DEAFNESS, AUTOSOMAL | others |
| | | DOMINANT | |
| | | NONSYNDROM1C | |
| | | SENSORINEURAL 2 | |
| GLUT1 | 6513 | Facilitated glucose transporter | others |
| | | protein type 1 (GLUT1) | |
| | | deficiency syndrome | |
| MYH | 4595 | hepatocellular carcinoma and | cancer/ |
| | | cholangiocarcinoma | tumorigenesis |
| FOXE3 | 2301 | anterior segment dysgenesis | others |
| | | similar to Peters' anomaly | |
| HUD | 1996 | poor prognosis | others |
| INK4C | 1031 | medulloblastoma formation | cancer/ |
| | | | tumorigenesis |
| NFIA | 4774 | Complex central nervous | mental |
| | | system (CNS) malformations | retardation |
| | | and urinary tract defects | others |
| CCN1 | 3491 | delayed formation of the | |
| | | ventricular septum in the | |
| | | embryo and persistent ostium | |
| | | primum atrial septal defects | |
| ABCA4 | 24 | Stargardt disease, retinitis | others |
| | | pigmentosa-19, and macular | |
| | | degeneration age-related 2 | |
| WNT2B | 7482 | mental retardation, short | mental |
| | | stature and colobomata | retardation |
| ADAR | 103 | dyschromatosis symmetrica | others |
| | | hereditaria | |
| ATP1A2 | 477 | familial hemiplegic migraine | others |
| | | type 2 | |
| MPZ | 4359 | neurologic diseases, including | neurological |
| | | CHN, DSS, and CMT1B | disorders |
| MYOC | 4653 | hereditary juvenile-onset open- | others |
| | | angle glaucoma | |
| HRPT2 | 79577 | Ossifying fibroma (progressive | others |
| | | enlargement of the affected | |
| | | jaw) | |
| LRH-1 | 2494 | inflammatory bowel disease | others |
| IRF6 | 3664 | van der Woude syndrome and | others |
| | | popliteal pterygium syndrome | |
| PROX1 | 5629 | Lymphatic vascular defects, | others |
| | | adult-onset obesity | |
| TP53BP2 | 7159 | no suppression of tumor | cancer/ |
| | | growth | tumorigenesis |
| NLRP3 | 114548 | CINCA syndrome | others |
| ID2 | 3398 | Congenital hydronephrosis | others |
| MYCN | 4613 | reduced brain size and | mental |
| | | intestinal atresias in Feingold | retardation |
| | | syndrome | |
| GCKR | 2646 | one form of maturity onset | others |
| | | diabetes of the young | |
| SPAST | 6683 | SPASTIC PARAPLEGIA 4 | others |
| MSH6 | 2956 | limitation of mismatch repair | others |
| FSHR | 2492 | degenerative changes in the | neurological |
| | | central nervous system | disorders |
| SPR | 6697 | dopa-responsive dystonia | neurological |
| | | | disorders |
| PAX8 | 7849 | congenital hypothyroidism | others |
| SMADIP1 | 9839 | syndromic Hirschsprung | others |
| | | disease | |
| RPRM | 56475 | tumorigenesis, no suppression | cancer/ |
| | | of tumor growth | tumorigenesis |
| SCN1A | 6323 | Severe myoclonic epilepsy of | others |
| | | infancy (SMEI) or Dravet | |
| | | syndrome | |
| HOXD13 | 3239 | foot malformations | others |
| COL3A1 | 1281 | Ehlers-Danlos syndrome type | others |
| | | IV, and with aortic and arterial | |
| | | aneurysms | |
| SLC40A1 | 30061 | ferroportin disease | others |
| SATB2 | 23314 | craniofacial dysmorphologies, | others |
| | | cleft palate | |
| SUMO1 | 7341 | nonsyndromic cleft lip and | others |
| | | palate | |
| BMPR2 | 659 | primary pulmonary | others |
| | | hypertension | |
| XRCC5 | 7520 | retarded growth, increased | growth |
| | | radiosensitivity, elevated p53 | retardation |
| | | levels and shortened telomeres | |
| PAX3 | 5077 | developmental delay and | growth |
| | | autism | retardation/ |
| | | | mental |
| | | | retardation |
| STK25 | 10494 | mild-to-moderate mental | mental |
| | | retardation with an Albright | retardation |
| | | hereditary osteodystrophy-like | |
| | | phenotype | |
| CHL1 | 10752 | 3p deletion (3p-) syndrome | unknown |
| SRGAP3 | 9901 | severe mental retardation | mental |
| | | | retardation |
| VHL | 7428 | increased lung cancer | cancer/ |
| | | susceptibility | tumorigenesis |
| GHRL | 51738 | GHRELIN | others |
| | | POLYMORPHISM | |
| PPARG | 5468 | susceptibility to mammary, | cancer/ |
| | | ovarian and skin | tumorigenesis |
| | | carcinogenesis | |
| SRG3 | 6599 | proteasomal degradation | others |
| RASSF1A | 11186 | pathogenesis of a variety of | cancer/ |
| | | cancers, no suppression of | tumorigenesis |
| | | tumor growth | |
| TKT | 7086 | reduced adipose tissue and | others |
| | | female fertility | |
| MITF | 4286 | Waardenburg syndrome type 2 | others |
| FOXP1 | 27086 | tumors development | cancer/ |
| | | | tumorigenesis |
| ROBO1 | 6091 | predispose to dyslexia | mental |
| | | | retardation |
| DIRC2 | 84925 | onset of tumor growth | cancer/ |
| | | | tumorigenesis |
| ATP2C1 | 27032 | orthodisease, skin disorder | others |
| FOXL2 | 668 | blepharophimosis syndrome | others |
| | | associated with ovarian | |
| | | dysfunction | |
| ATR | 545 | mismatch repair-deficient | others |
| SI | 6476 | SUCRASE-ISOMALTASE | others |
| | | DEFICIENCY, | |
| | | CONGENITAL | |
| TERC | 7012 | Autosomal dominant | others |
| | | dyskeratosis congenita | |
| | | (AD DC), a rare inherited bone | |
| | | marrow failure syndrome | |
| SOX2 | 6657 | hippocampal malformations | neurological |
| | | and epilepsy | disorders |
| OPA1 | 4976 | optic atrophy | others |
| TFRC | 7037 | stressed erythropoiesis and | neurological |
| | | neurologic abnormalities | disorders |
| FGFR3 | 2261 | a variety of skeletal dysplasias, | others |
| | | including the most common | |
| | | genetic form of dwarfism, | |
| | | achondroplasia | |
| LETM1 | 3954 | Wolf Hirshhorn syndrome | mental |
| | | | retardation |
| SH3BP2 | 6452 | Wolf-Hirshhorn syndrome | mental |
| | | | retardation |
| MSX1 | 4487 | oligodontia | others |
| RBPJ | 3516 | embryonic lethality and | others |
| | | formation of arteriovenous | |
| | | malformations | |
| PHOX2B | 8929 | predispose to Hirschsprung | neurological |
| | | disease | disorders |
| ENAM | 10117 | Amelogenesis imperfecta | others |
| | | (inherited defects of dental | |
| | | enamel formation) | |
| MAPK10 | 5602 | epileptic encephalopathy of the | neurological |
| | | Lennox-Gaustaut type | disorders |
| PKD2 | 5311 | Autosomal dominant | others |
| | | polycystic kidney disease | |
| SNCA | 6622 | familial Parkinson's disease | neurological |
| | | | disorders |
| RIEG | 5308 | Rieger syndrome (RIEG) | others |
| | | characterized by | |
| | | malformations of the anterior | |
| | | segment of the eye, failure of | |
| | | the periumbilical skin to | |
| | | involute, and dental hypoplasia | |
| ANK2 | 287 | arrhythmia | others |
| MAD2L1 | 4085 | optimal hematopoiesis | others |
| PLK4 | 10733 | mitotic infidelity and | cancer/ |
| | | carcinogenesis | tumorigenesis |
| FBXW7 | 55294 | cancer (breast, ovary) tumors | cancer/ |
| | | development | tumorigenesis |
| TERT | 7015 | DYSKERATOSIS | others |
| | | CONGENITA | |
| SEMA5A | 9037 | abnormal brain development | mental |
| | | | retardation |
| GDNF | 2668 | complex human diseases | others |
| | | (Hirschsprung-like intestinal | |
| | | obstruction and early-onset | |
| | | lethality) | |
| FGF10 | 2255 | craniofacial development and | developmental |
| | | developmental disorders | abnormalities |
| PIK3R1 | 5295 | insulin resistance | others |
| APC | 324 | familial adenomatous | cancer/ |
| | | polyposis | tumorigenesis |
| RAD50 | 10111 | hereditary breast cancer | cancer/ |
| | | susceptibility associated with | tumorigenesis |
| | | genomic instability | |
| SMAD5 | 4090 | secondary myelodysplasias and | cancer/ |
| | | acute myeloid leukemias | tumorigenesis |
| EGR1 | 1958 | development of myeloid | others |
| | | disorders | |
| TCOF1 | 6949 | depletion of neural crest cell | developmental |
| | | precursors, Treacher Collins | abnormalities |
| | | syndrome | |
| NPM1 | 4869 | myelodysplasias and leukemias | cancer/ |
| | | | tumorigenesis |
| NKX2-5 | 1482 | microcephaly and congenital | others |
| | | heart disease | |
| MSX2 | 4488 | pleiotropic defects in bone | others |
| | | growth and ectodermal organ | |
| | | formation | |
| NSD1 | 64324 | Sotos syndrome | mental |
| | | | retardation |
| FOXC1 | 2296 | Axenfeld-Rieger anomaly of | others |
| | | the anterior eye chamber | |
| DSP | 1832 | skin fragility/woolly hair | others |
| | | syndrome; disruption of tissue | |
| | | structure, integrity and changes | |
| | | in keratinocyte proliferation | |
| EEF1E1 | 9521 | no suppression of tumor | cancer/ |
| | | growth | tumorigenesis |
| TNXA | 7146 | Ehlers-Danlos syndrome | others |
| TKX | 7148 | Elastic fiber abnormalities in | others |
| | | hypermobility type Ehlers- | |
| | | Danlos syndrome | |
| HMGA1 | 3159 | insulin resistance and diabetes | others |
| RUNX2 | 860 | cleidocranial dysplasia | developmental |
| | | | abnormalities |
| CD2AP | 23607 | glomerular disease | others |
| | | susceptibility | |
| ELOVL4 | 6785 | defective skin permeability | others |
| | | barrier function and neonatal | |
| | | lethality | |
| NT5E | 4907 | Neuropathy target esterase | neurological |
| | | deficiency | disorders |
| SIM1 | 6492 | impaired melanocortin- | others |
| | | mediated anorexia and | |
| | | activation of paraventricular | |
| | | nucleus neurons | |
| COL10A1 | 1300 | Schmid type metaphyseal | others |
| | | chondrodysplasia and Japanese | |
| | | type spondylometaphyseal | |
| | | dysplasia | |
| PARK2 | 5071 | PARKINSON DISEASE 2 | neurological |
| | | | disorders |
| TWIST1 | 7291 | coronal synostosis | developmental |
| | | | abnormalities |
| GLI3 | 2737 | Greig cephalopolysyndactyly | developmental |
| | | and Pallister-Hall syndromes | abnormalities |
| GCK | 2645 | non-insulin dependent diabetes | others |
| | | mellitus (NIDDM), maturity | |
| | | onset diabetes of the young, | |
| | | type 2 (MODY2) and | |
| | | persistent hyperinsulinemic | |
| | | hypoglycemia of infancy | |
| | | (PHHI) | |
| FKBP6 | 8468 | Williams-Beuren syndrome | mental |
| | | | retardation |
| ELN | 2006 | cardiovascular disease and | others |
| | | connective tissue abnormalities | |
| LIMK1 | 3984 | Williams syndrome (WS), a | mental |
| | | neurodevelopmental disorder | retardation |
| RFC 2 | 5982 | growth deficiency as well as | growth |
| | | developmental disturbances in | retardation |
| | | Williams syndrome | |
| GTF3 | 9569 | abnormal muscle fatiguability | others |
| GTF2I | 2969 | Williams-Beuren syndrome | mental |
| | | | retardation |
| NCF1 | 653361 | autosomal recessive chronic | others |
| | | granulomatous disease | |
| KRIT1 | 889 | Cerebral Cavernous | others |
| | | Malformations (vascular | |
| | | malformations characterized | |
| | | by abnormally enlarged | |
| | | capillary cavities) | |
| COL1A2 | 1278 | subtle symptoms like recurrent | others |
| | | joint subluxation or hypodontia | |
| SHFM1 | 7979 | severe mental retardation, short | mental |
| | | stature, microcephaly and | retardation |
| | | deafness | |
| RELN | 5649 | Cognitive disruption and | others |
| | | altered hippocampus synaptic | |
| | | function | |
| FOXP2 | 93986 | Speech and language | mental |
| | | impairment and oromotor | retardation |
| | | dysprax | |
| CAV1 | 857 | 17beta-estradiol-stimulated | cancer/ |
| | | mammary tumorigenesis | tumorigenesis |
| ST7 | 7982 | no suppression of tumor | cancer/ |
| | | growth | tumorigenesis |
| BRAF | 673 | Cardiofaciocutaneous (CFC) | growth and |
| | | syndrome | mental |
| | | | retardation |
| SHH | 6469 | Holoprosencephaly, sacral | others |
| | | anomalies, and situs ambiguus | |
| HLXB9 | 3110 | Currarino syndrome including | others |
| | | a presacral mass, sacral | |
| | | agenesis, and anorectal | |
| | | malformation | |
| GATA4 | 2626 | congenital heart disease | others |
| NKX3-1 | 4824 | prostate cancer | cancer/ |
| | | | tumorigenesis |
| FGFR1 | 2260 | Pfeiffer syndrome, Jackson- | others |
| | | Weiss syndrome, Antley- | |
| | | Bixler syndrome, | |
| | | osteoglophonic dysplasia, and | |
| | | autosomal dominant Kallmann | |
| | | syndrome 2 | |
| CHD7 | 55636 | CHARGE syndrome | growth |
| | | | retardation |
| CSN5 | 10987 | TRC8 hereditary kidney cancer | cancer/ |
| | | | tumorigenesis |
| EYA1 | 2138 | branchiootorenal dysplasia | others |
| | | syndrome, branchiootic | |
| | | syndrome, and sporadic cases | |
| | | of congenital cataracts and | |
| | | ocular anterior segment | |
| | | anomalies | |
| TRPS1 | 7227 | dominantly inherited tricho- | growth |
| | | rhino-phalangeal (TRP) | retardation |
| | | syndromes | |
| DMRT1 | 1761 | failure of testicular | others |
| | | development and feminization | |
| | | in male | |
| DMRT2 | 10655 | defective testis formation in | others |
| | | karyotypic males and impaired | |
| | | ovary function in karyotypic | |
| | | females | |
| MLLT3 | 4300 | neuromotor developmental | neurological |
| | | delay, cerebellar ataxia, and | disorders |
| | | epilepsy | |
| ARF | 1029 | acute myeloid leukemia | cancer/ |
| | | | tumorigenesis |
| CDKN2B | 1030 | syndrome of cutaneous | cancer/ |
| | | malignant melanoma and | tumorigenesis |
| | | nervous system tumors | |
| BAG1 | 573 | lung tumorigenesis | tumorigenesis |
| PAX5 | 5079 | pathogenesis of lymphocytic | cancer/ |
| | | lymphomas | tumorigenesis |
| GCNT1 | 2650 | T lymphoma cells resistant to | others |
| | | cell death | |
| ROR2 | 4920 | basal cell nevus syndrome | cancer/ |
| | | (BCNS) | tumorigenesis |
| PTCH1 | 5727 | Primitive neuroectodermal | cancer/ |
| | | nimors formation | tumorigenesis |
| NR5A1 | 2516 | impaired testicular | others |
| | | development, sex reversal, and | |
| | | adrenal failure | |
| LMX1B | 4010 | nail-patella syndrome | developmental |
| | | | abnormalities |
| ENG | 2022 | Hereditary hemorrhagic | developmental |
| | | telangiectasia type 1 | abnormalities |
| TSC1 | 7248 | transitional cell carcinoma of | cancer/ |
| | | the bladder | tumorigenesis |
| COL5A1 | 1289 | Structural abnormalities of the | developmental |
| | | cornea and lid | abnormalities |
| NOTCH1 | 4851 | aortic valve disease (cardiac | others |
| | | malformation and aortic valve | |
| | | calcification) | |
| EHMT1 | 79813 | 9q34 subtelomeric deletion | mental |
| | | syndrome | retardation |
| KLF6 | 1316 | cellular growth dysregulation | cancer/ |
| | | and tumorigenesis | tumorigenesis |
| GATA3 | 2625 | HDR (hypoparathyroidism, | others |
| | | deafness and renal dysplasia) | |
| | | syndrome | |
| ANX7 | 310 | tumorigenesis | cancer/ |
| | | | tumorigenesis |
| PTEN | 5728 | prostate cancer high-grade | cancer/ |
| | | prostatic intra-epithelial | tumorigenesis |
| | | neoplasias | |
| PAX2 | 5076 | renal-coloboma syndrome | others |
| FGF8 | 2253 | several human craniofacial | others |
| | | disorders | |
| BUB3 | 9184 | short life span that is | others |
| | | associated with the early onset | |
| | | of aging-related feanires | |
| CDKN1C | 1028 | Beckwith-Wiedemann | cancer/ |
| | | syndrome | tumorigenesis |
| NUP98 | 4928 | destruction of securin in | others |
| | | mitosis | |
| PAX6 | 5080 | eye diseases | others |
| WT1 | 7490 | congenital genitourinary (GU) | cancer/ |
| | | anomalies and or bilateral | tumorigenesis |
| | | disease and tumorigenesis | |
| EXT2 | 2132 | type II form of multiple | developmental |
| | | exostoses | abnormalities |
| ALX4 | 60529 | Tibial aplasia, lower extremity | others |
| | | mirror image polydactyly, | |
| | | brachyphalangy, craniofacial | |
| | | dysmorphism and genital | |
| | | hypoplasia | |
| FEN1 | 2237 | neuromuscular and | mental |
| | | neurodegenerative diseases | retardation |
| SF1 | 7536 | mild gonadal dysgenesis and | others |
| | | impaired androgenization | |
| FGF3 | 2248 | otodental syndrome | others |
| FZD4 | 8322 | complex chromosome | growth |
| | | rearrangement with multiple | retardation |
| | | abnormalities including growth | |
| | | retardation, facial anomalies, | |
| | | exudative vitreoretinopathy | |
| | | (EVR), cleft palate, and minor | |
| | | digital anomalies | |
| ATM | 472 | High incidence of cancer | cancer/ |
| | | | tumorigenesis |
| H2AX | 3014 | genomic instability, early onset | cancer/ |
| | | of various tumors | tumorigenesis |
| FLI1 | 2313 | Paris-Trousseau thrombopenia | others |
| NFRKB | 4798 | cellular immunodeficiency, | others |
| | | pancytopenia, malformations | |
| PHB2 | 11331 | enhanced estrogen receptor | others |
| | | function | |
| ETV6 | 2120 | a pediatric pre-B acute | cancer/ |
| | | lymphoblastic leukemia | tumorigenesis |
| CDKN1B | 1027 | ErbB2-induced mammary | cancer/ |
| | | nimor growth | tumorigenesis |
| COL2A1 | 1280 | Stickler syndrome | others |
| KRT5 | 3852 | epidermolysis bullosa simplex | others |
| MYF6 | 4618 | myopathy and severe course of | others |
| | | Becker muscular dystrophy | |
| IGF1 | 3479 | subtle inhibition of intrauterine | others |
| | | and postnatal growth | |
| SERCA2 | 488 | colon and lung cancer | cancer/ |
| | | | tumorigenesis |
| TBX5 | 6910 | maturation failure of | others |
| | | conduction system morphology | |
| | | and function in Holt-Oram | |
| | | syndrome | |
| TBX3 | 6926 | ulnar-mammary syndrome | others |
| HNF1A | 6927 | reduced serum apolipoprotein | others |
| | | M levels | |
| BRCA2 | 675 | predisposed to breast, ovarian, | cancer/ |
| | | pancreatic and other cancers | tumorigenesis |
| FKHR | 2308 | Alveolar rhabdomyosarcomas | others |
| RB1 | 5925 | Metaphase cytogenetic | others |
| | | abnormalities | |
| ZIC2 | 7546 | neurological disorders, | mental |
| | | behavioral abnormalities | retardation |
| LIG4 | 3981 | LIG4 syndrome, nonlymphoid | cancer/ |
| | | tumorigenesis | tumorigenesis |
| COCH | 1690 | unknown | unknown |
| NPAS3 | 64067 | schizophrenia | neurological |
| | | | disorders |
| NKX2-1 | 7080 | Choreoathetosis, | neurological |
| | | hypothyroidism, pulmonary | disorders |
| | | alterations, neurologic | |
| | | phenotype and secondary | |
| | | hyperthyrotropinemia, and | |
| | | diseases due to transcription | |
| | | factor defects | |
| PAX9 | 5083 | posterior tooth agenesis | others |
| BMP4 | 652 | a contiguous gene syndrome | others |
| | | comprising anophthalmia, | |
| | | pituitary hypoplasia, and ear | |
| | | anomalies | |
| GCH1 | 2643 | malignant | others |
| | | hyperphenylalaninemia and | |
| | | dopa-responsive dystonia | |
| SIX6 | 4990 | bilateral anophthalmia and | others |
| | | pituitary anomalies | |
| RAD51B | 5890 | centrosome fragmentation and | others |
| | | aneuploidy | |
| BCL11B | 64919 | suppression of | others |
| | | lymphomagenesis and | |
| | | thymocyte development | |
| SPRED1 | 161742 | neurofibromatosis type 1-like | cancer/ |
| | | syndrome | tumorigenesis |
| BUBR1 | 701 | enhanced tumor development | cancer/ |
| | | | tumorigenesis |
| DLL4 | 54567 | embryonic lethality due to | others |
| | | major defects in arterial and | |
| | | vascular development | |
| FBN1 | 2200 | Marfan syndrome, isolated | others |
| | | ectopia lentis, autosomal | |
| | | dominant Weill-Marchesani | |
| | | syndrome, MASS syndrome, | |
| | | and Shprintzen-Goldberg | |
| | | craniosynostosis syndrome | |
| ALDH1A2 | 8854 | facilitate posterior organ | others |
| | | development and prevent spina | |
| | | bifida | |
| TPM1 | 7168 | type 3 familial hypertrophic | others |
| | | cardiomyopathy | |
| P450SCC | 1583 | 46,XY sex reversal and adrenal | others |
| | | insufficiency | |
| BLM | 641 | the autosomal recessive | mental |
| | | disorder Bloom syndrome | retardation |
| COUP- | 7026 | several malformations, pre- | growth |
| TFII | | and postnatal growth | retardation |
| | | retardation and developmental | |
| SOX8 | 30812 | the mental retardation found in | mental |
| | | ATR-16 syndrome | retardation |
| TSC2 | 7249 | the differential cancer | cancer/ |
| | | susceptibility | tumorigenesis |
| PKD1 | 5310 | autosomal dominant polycystic | others |
| | | kidney disease | |
| CBP | 1387 | Rubinstein-Taybi syndrome | mental |
| | | | retardation |
| SOCS1 | 8651 | severe liver fibrosis and | cancer/ |
| | | hepatitis-induced | tumorigenesis |
| | | carcinogenesis | |
| PRM2 | 5620 | infertility | others |
| PRM1 | 5619 | infertility | others |
| ABCC6 | 368 | pseudoxanthoma elasticum | others |
| ERAF | 51327 | subtle erythroid phenotype | others |
| SALL1 | 6299 | Townes-Brocks syndrome | developmental |
| | | | abnormalities/ |
| | | | mental |
| | | | retardation |
| CBFB | 865 | delayed cranial ossification, | others |
| | | cleft palate, congenital heart | |
| | | anomalies, and feeding | |
| | | difficulties | |
| CTCF | 10664 | loss of imprinting of insulin- | cancer/ |
| | | like growth factor-II in Wilms | tumorigenesis |
| | | tumor | |
| WWOX | 51741 | initiation of tumor | cancer/ |
| | | development | tumorigenesis |
| FOXF1 | 2294 | defects in formation and | others |
| | | branching of primary lung | |
| | | buds | |
| FOXC2 | 2303 | the lymphatic/ocular disorder | others |
| | | Lymphedema-Distichiasis | |
| YWHAE | 7531 | pathogenesis of small cell lung | cancer/ |
| | | cancer | tumorigenesis |
| HIC1 | 3090 | Miller-Dieker syndrome | growth and |
| | | | mental |
| | | | retardation |
| LIS1 | 5048 | abnormal cell proliferation, | cancer/ |
| | | migration and differentiation in | tumorigenesis |
| | | the adult dentate gyrus | |
| P53 | 7157 | male oral squamous cell | cancer/ |
| | | carcinomas | tumorigenesis |
| PMP22 | 5376 | hereditary neuropathy with | neurological |
| | | liability to pressure palsies | disorders |
| COPS3 | 8533 | Circadian rhythm | others |
| | | abnormalities of melatonin in | |
| | | Smith-Magenis syndrome | |
| RAI1 | 10743 | Smith-Magenis syndrome | mental |
| | | | retardation |
| TOP3A | 7156 | Smith-Magenis syndrome | mental |
| | | | retardation |
| SHMT1 | 6470 | Smith-Magenis syndrome | mental |
| | | | retardation |
| RNF135 | 84282 | phenotypic abnormalities | others |
| | | including overgrowth | |
| NF1 | 4763 | neurofibromatosis type 1 | mental |
| | | | retardation |
| SUZ12 | 23512 | mental impairment in | mental |
| | | constitutional NF1 | retardation |
| | | microdeletions | |
| MEL-18 | 7703 | breast carcinogenesis | cancer/ |
| | | | tumorigenesis |
| KLHL10 | 317719 | disrupted spermiogenesis | others |
| STAT5B | 6777 | striking amelioration of IL-7- | others |
| | | induced mortality and disease | |
| | | development | |
| STAT5A | 6776 | striking amelioration of IL-7- | others |
| | | induced mortality and disease | |
| | | development | |
| BECN1 | 8678 | autophagy function, and tumor | cancer/ |
| | | suppressor function | tumorigenesis |
| BRCA1 | 672 | shortened life span and ovarian | cancer/ |
| | | tumorigenesis | tumorigenesis |
| PGRN | 2896 | neurodegeneration | mental |
| | | | retardation |
| MAPT | 4137 | neuronal cell death, | neurological |
| | | neurodegenerativec disorders | disorders |
| | | such as Alzheimer's disease, | |
| | | Pick's disease, frontotemporal | |
| | | dementia, cortico-basal | |
| | | degeneration and progressive | |
| | | supranuclear palsy | |
| CSH1 | 1442 | Silver-Russell svndrome | others |
| POLG2 | 11232 | mtDNA deletions causes COX | others |
| | | deficiency in muscle fibers and | |
| | | results in the clinical | |
| | | phenotype | |
| PRKAR1A | 5573 | Carney complex, a familial | cancer/ |
| | | multiple neoplasia syndrome | tumorigenesis |
| SOX9 | 6662 | skeletal dysplasias | cancer/ |
| | | | tumorigenesis |
| NHERF1 | 9368 | breast tumors | cancer/ |
| | | | tumorigenesis |
| FSCN2 | 25794 | photoreceptor degeneration, | others |
| | | autosomal dominant retinitis | |
| | | pigmentosa | |
| DSG1 | 1828 | diseases of epidermal integrity | others |
| DSG2 | 1829 | ARRHYTHMOGENIC | others |
| | | RIGHT VENTRICULAR | |
| | | DYSPLASIA | |
| TCF4 | 6925 | Pitt-Hopkins syndrome, a | mental |
| | | syndromic mental disorder | retardation |
| FECH | 2235 | protoporphyria | others |
| MC4R | 4160 | increased adiposity and linear | others |
| | | growth | |
| GALR1 | 2587 | uncontrolled proliferation and | others |
| | | neoplastic transformation | |
| SALL3 | 27164 | 18q deletion syndrome | others |
| LKB1 | 6794 | Peutz-Jeghers syndrome | cancer/ |
| | | | tumorigenesis |
| PNPLA6 | 10908 | organophosphorus-induced | others |
| | | hyperactivity and toxicity | |
| RYR1 | 6261 | malignant hyperthermia | others |
| | | susceptibility, central core | |
| | | disease, and minicore | |
| | | myopathy with external | |
| | | ophthalmoplegia | |
| TGFB1 | 7040 | Aggressive pancreatic ductal | cancer/ |
| | | adenocarcinoma | tumorigenesis |
| RPS19 | 6223 | Diamond-Blackfan anemia | others |
| DMPK | 1760 | cardiac disease in myotonic | others |
| | | dystrophy | |
| CRX | 1406 | photoreceptor degeneration. | others |
| | | Leber congenital amaurosis | |
| | | type III and the autosomal | |
| | | dominant cone-rod dystrophy 2 | |
| PRPF31 | 26121 | retinitis pigmentosa with | others |
| | | reduced penetrance | |
| JAG1 | 182 | Alagille syndrome | mental |
| | | | retardation |
| PAX1 | 5075 | Klippel-Feil syndrome | others |
| GDF5 | 8200 | Multiple-synostosis syndrome | mental |
| | | | retardation |
| HNF4A | 3172 | monogenic autosomal | others |
| | | dominant non-insulin- | |
| | | dependent diabetes mellitus | |
| | | type I | |
| SALL4 | 57167 | Okihiro syndrome | developmental |
| | | | abnormalities |
| MC3R | 4159 | susceptibility to obesity | others |
| RAE1 | 8480 | premature separation of sister | others |
| | | chromatids, severe aneuploidy | |
| | | and untimely degradation of | |
| | | securin | |
| GNAS | 2778 | reduced activation of a | others |
| | | downstream target in epithelial | |
| | | tissues | |
| EDN3 | 1908 | Hirschsprung disease | others |
| KCNQ2 | 3785 | epilepsy susceptibility | neurological |
| | | | disorders |
| SOX18 | 54345 | mental retardation | mental |
| | | | retardation |
| SLC5A3 | 6526 | brain inositol deficiency | others |
| RUNX1 | 861 | The 8p11 myeloproliferative | others |
| | | syndrome | |
| DYRK1A | 1859 | neurological defects, | growth |
| | | developmental delay | retardation |
| | | | mental |
| | | | retardation |
| COL6A1 | 1291 | autosomal dominant disorder, | neurological |
| | | Bethlem myopathy | disorders |
| PRODH | 5625 | 22q11 Deletion syndrome | mental |
| | | | retardation |
| DGCR2 | 9993 | DiGeorge syndrome | mental |
| | | | retardation |
| HIRA | 7290 | DiGeorge syndrome (cranio- | mental |
| | | facial, cardiac and thymic | retardation |
| | | malformations) | |
| TBX1 | 6899 | 22q11 deletion syndrome and | mental |
| | | schizophrenia | retardation |
| COMT | 1312 | 22q11.2 deletion syndrome | mental |
| | | | retardation |
| RTN4R | 65078 | schizophrenia susceptibility | others |
| | | (schizoaffective disorders are | |
| | | common features in patients | |
| | | with DiGeorge/ | |
| | | velocardiofacial syndrome) | |
| PCQAP | 51586 | DiGeorge syndrome | others |
| LZTR1 | 8216 | DiGeorge syndrome | mental |
| | | | retardation |
| INI1 | 6598 | pituitary tumorigenesis | cancer/ |
| | | | tumorigenesis |
| MYH9 | 4627 | hematological abnormalities | others |
| SOX10 | 6663 | the etiology of Waardenburg/ | others |
| | | Hirschsprung disease | |
| FBLM | 2192 | limb malformations | others |
| PPARA | 5465 | prostate cancer | cancer/ |
| | | | tumorigenesis |
| PROSAP2 | 85358 | The terminal 22q13.3 deletion | mental |
| | | syndrome, characterized by | retardation |
| | | severe expressive-language | |
| | | delay, mild mental retardation, | |
| | | hypotonia, joint laxity, | |
| | | dolichocephaly, and minor | |
| | | facial dysmorphisms | |
| SHOX | 6473 | congenital form of growth | growth |
| | | failure, the etiology of | retardation |
| | | ″idiopathic″ short stature and | |
| | | the growth deficits and skeletal | |
| | | anomalies in Leri Weill, | |
| | | Langer and Turner syndrome | |
| P2RY8 | 286530 | mentally retarded males | mental |
| | | | retardation |
| NLGN4X | 57502 | autism and Asperger syndrome | neurological |
| | | | disorders |
| TRAPPC2 | 6399 | spondyloepiphyseal dysplasia | cancer/ |
| | | tarda | tumorigenesis |
| RPS4X | 6191 | unknown | unknown |
| CSF2RA | 1438 | growth deficiency | growth |
| | | | retardation |

Any statistically significant increase in expression of the target gene can be achieved. For example, the increase in expression of the target gene can be at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, or at least about 20-fold higher relative to a control eukaryotic genome, cell, or organism (e.g., as measured at the RNA level or the protein level). Likewise, the duration of the increase in expression of the target gene can be for any suitable time. For example, the duration of the increase in expression of the target gene can be for at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, or at least about 2 months. In a specific example, the increase in expression of the target gene at 1 day, 2 days, 3 days, 4 days, 5 days, 6 days, 1 week, 2 weeks, 3 weeks, 4 weeks, or 1 month following introducing of the CRISPR/Cas synergistic activation mediator (SAM) system can be at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, or at least about 20-fold higher relative to a control eukaryotic genome, cell, or organism. In one example, the increase is at least about 2-fold after 1, 2, or 3 weeks with a dose of 0.5 mg/kg LNP or 1 mg/kg LNP or 2 mg/kg LNP. In another example, the increase is at least about 3-fold after 1, 2, or 3 weeks with a dose of 0.5 mg/kg LNP or 1 mg/kg LNP or 2 mg/kg LNP. In another example, the increase is at least about 4-fold after 1, 2, or 3 weeks with a dose of 0.5 mg/kg LNP or 1 mg/kg LNP or 2 mg/kg LNP. In another example, the increase is at least about 5-fold after 1, 2, or 3 weeks with a dose of 0.5 mg/kg LNP or 1 mg/kg LNP or 2 mg/kg LNP. In another example, the increase is at least about 6-fold after 1, 2, or 3 weeks with a dose of 0.5 mg/kg LNP or 1 mg/kg LNP or 2 mg/kg LNP. In another example, the increase is at least about 7-fold after 1, 2, or 3 weeks with a dose of 0.5 mg/kg LNP or 1 mg/kg LNP or 2 mg/kg LNP. In another example, the increase is at least about 8-fold after 1, 2, or 3 weeks with a dose of 0.5 mg/kg LNP or 1 mg/kg LNP or 2 mg/kg LNP. In another example, the increase is at least about 9-fold after 1, 2, or 3 weeks with a dose of 0.5 mg/kg LNP or 1 mg/kg LNP or 2 mg/kg LNP. In another example, the increase is at least about 10-fold after 1, 2, or 3 weeks with a dose of 0.5 mg/kg LNP or 1 mg/kg LNP or 2 mg/kg LNP. The increase in expression of the target gene can be sustained at a near constant level (i.e., without showing a pattern of decreasing over time) for at least about 1 day, at least about 2 days, at least about 3 days, at least about 4 days, at least about 5 days, at least about 6 days, at least about 1 week, at least about 2 weeks, at least about 3 weeks, at least about 4 weeks, or more.

The methods can be for increasing transcription or expression of target genes in any eukaryotic genome, cell, or organism. The genomes, cells, or eukaryotic organisms (e.g., animal, non-human animal, mammal, or non-human mammal) can be male or female. In some methods, the transcription or expression of the target gene is increased in a subject (e.g., organism or animal or mammal, such as a human) in need thereof. For example, the subject in need thereof can be a subject with a disease, disorder, or syndrome associated with, exacerbated by, or caused by reduced transcription or expression of the target gene, reduced amount of the gene product of the target gene, or reduced activity of the gene product by the target gene, such that increasing transcription or expression of the target gene, increasing the amount of the gene product of the target gene, or increasing the activity of the gene product of the target gene would be beneficial. The target gene can be underexpressed or expressed at low levels in the subject relative to a control subject without the disease, disorder, or syndrome. For example, increasing transcription or expression of the target gene, increasing the amount of the gene product of the target gene, or increasing the activity of the gene product of the target gene could treat the disease, disorder, or syndrome in the subject. Examples of such diseases, disorders, or syndromes include disease, disorders, or syndromes associated with haploinsufficiency. Examples of haploinsufficient genes and other genes for which increasing transcription or expression would be beneficial are provided in Tables 2 and 3 and elsewhere herein.

The eukaryotic genomes, cells, or organisms provided herein can be, for example, multicellular eukaryotic, non-human eukaryotic, animal, non-human animal, mammalian, non-human mammalian, human, non-human, rodent, mouse, or rat genomes, cells, or organisms. Eukaryotic cells include, for example, fungal cells (e.g., yeast), plant cells, animal cells, mammalian cells, non-human mammalian cells, and human cells. The term “animal” includes mammals, fishes, and birds. Mammals include, for example, humans, non-human primates, monkeys, apes, cats, dogs, horses, bulls, deer, bison, sheep, rabbits, rodents (e.g., mice, rats, hamsters, and guinea pigs), and livestock (e.g., bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human animal” excludes humans.

Cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

Examples of human pluripotent cells include human ES cells, human adult stem cells, developmentally restricted human progenitor cells, and human induced pluripotent stem (iPS) cells, such as primed human iPS cells and naïve human iPS cells. Induced pluripotent stem cells include pluripotent stem cells that can be derived directly from a differentiated adult cell. Human iPS cells can be generated by introducing specific sets of reprogramming factors into a cell which can include, for example, Oct3/4, Sox family transcription factors (e.g., Sox1, Sox2, Sox3, Sox15), Myc family transcription factors (e.g., c-Myc, 1-Myc, n-Myc), Krüppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, e.g., Takahashi and Yamanaka (2006) Cell 126:663-676, herein incorporated by reference in its entirety for all purposes. Primed human ES cells and primed human iPS cells include cells that express characteristics similar to those of post-implantation epiblast cells and are committed for lineage specification and differentiation. Naïve human ES cells and naïve human iPS cells include cells that express characteristics similar to those of ES cells of the inner cell mass of a pre-implantation embryo and are not committed for lineage specification. See, e.g., Nichols and Smith (2009) Cell Stem Cell 4:487-492, herein incorporated by reference in its entirety for all purposes.

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, kidney cells, hematopoietic cells, endothelial cells, epithelial cells, fibroblasts, mesenchymal cells, keratinocytes, blood cells, melanocytes, monocytes, mononuclear cells, monocytic precursors, B cells, erythroid-megakaryocytic cells, eosinophils, macrophages, T cells, islet beta cells, exocrine cells, pancreatic progenitors, endocrine progenitors, adipocytes, preadipocytes, neurons, glial cells, neural stem cells, neurons, hepatoblasts, hepatocytes, cardiomyocytes, skeletal myoblasts, smooth muscle cells, ductal cells, acinar cells, alpha cells, beta cells, delta cells, PP cells, cholangiocytes, white or brown adipocytes, or ocular cells (e.g., trabecular meshwork cells, retinal pigment epithelial cells, retinal microvascular endothelial cells, retinal pericyte cells, conjunctival epithelial cells, conjunctival fibroblasts, iris pigment epithelial cells, keratocytes, lens epithelial cells, non-pigment ciliary epithelial cells, ocular choroid fibroblasts, photoreceptor cells, ganglion cells, bipolar cells, horizontal cells, or amacrine cells). For example, the cells can be liver cells, such as hepatoblasts or hepatocytes.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, somatic cells, hematopoietic cells, endothelial cells, epithelial cells, fibroblasts, mesenchymal cells, keratinocytes, melanocytes, monocytes, mononuclear cells, adipocytes, preadipocytes, neurons, glial cells, hepatocytes, skeletal myoblasts, and smooth muscle cells. For example, primary cells can be derived from connective tissues, muscle tissues, nervous system tissues, or epithelial tissues. Such cells can be isolated by conventional techniques and include, for example, hepatocytes.

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Examples of immortalized cells include Chinese hamster ovary (CHO) cells, human embryonic kidney cells (e.g., HEK 293 cells or 293T cells), and mouse embryonic fibroblast cells (e.g., 3T3 cells). A specific example of an immortalized cell line is the HepG2 human liver cancer cell line. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos (e.g., rodent one-cell stage embryos) can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

The eukaryotic genomes, cells, or organisms can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Sv1m), 129S2, 129S4, 129S5, 12959/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Ka1_wN, C57BL/6, C57BL/6J, C57BL/6ByJ, C57BL/6NJ, C57BL/10, C57BL/10ScSn, C57BL/10Cr, and C57BL/01a. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. In some cases, suitable rats can be from an inbred rat strain. See, e.g., US 2014/0235933, herein incorporated by reference in its entirety for all purposes.

Various methods are also provided for optimizing delivery of a CRISPR/Cas SAM system to a cell or eukaryotic organism (e.g., animal, non-human animal, mammal, or non-human mammal) or optimizing CRISPR/Cas transcriptional activation activity in vivo or ex vivo. Such methods can comprise, for example: (a) performing the method of testing the ability of a CRISPR/Cas SAM system to increase transcription or expression a target gene as described above a first time in a first eukaryotic organism (e.g., animal, non-human animal, mammal, or non-human mammal) or first cell; (b) changing a variable and performing the method a second time in a second eukaryotic organism (e.g., animal, non-human animal, mammal, or non-human mammal; i.e., of the same species) or a second cell with the changed variable; and (c) comparing expression/transcription of the target gene in step (a) with the expression/transcription of the target gene in step (b), and selecting the method resulting in the highest expression/transcription of the target gene.

Alternatively or additionally, the method resulting in the highest efficacy, highest consistency, or highest specificity can be chosen. Higher efficacy refers to higher levels of expression/transcription of the target gene (e.g., a higher percentage of cells is targeted within a particular target cell type, within a particular target tissue, or within a particular target organ). Higher consistency refers to more consistent increases in expression/transcription of the target gene among different types of targeted cells, tissues, or organs if more than one type of cell, tissue, or organ is being targeted (e.g., increased expression/transcription of a greater number of cell types within a target organ). If a particular organ is being targeted, higher consistency can also refer to more consistent increases in expression/transcription throughout all locations within the organ. Higher specificity can refer to higher specificity with respect to the target gene or genes being targeted, higher specificity with respect to the cell type targeted, higher specificity with respect to the tissue type targeted, or higher specificity with respect to the organ targeted. For example, increased target specificity refers to fewer off-target effects on other genes (e.g., a lower percentage of targeted cells having increased transcription at unintended, off-target genomic loci (e.g., neighboring genomic loci) instead of, or in addition to, increased transcription of the target gene). Likewise, increased cell type, tissue, or organ type specificity refers to fewer effects (i.e., increased expression/transcription) in off-target cell types, tissue types, or organ types if a particular cell type, tissue type, or organ type is being targeted (e.g., when a particular organ is targeted (e.g., the liver), there are fewer effects (i.e., increased expression/transcription) in cells in organs or tissues that are not intended targets).

The variable that is changed can be any parameter. As one example, the changed variable can be the route of administration for introduction of SAM components (chimeric Cas protein, chimeric adaptor protein, and guide RNA(s)) into the cell or eukaryotic organism (e.g., animal, non-human animal, mammal, or non-human mammal). Examples of routes of administration, such as intravenous, intravitreal, intraparenchymal, and nasal instillation, are disclosed elsewhere herein.

As another example, the changed variable can be the concentration or amount of the SAM components introduced. As another example, the changed variable can be the number of times or frequency with which the SAM components are introduced (i.e., the number of times or frequency with which the LNP is introduced). As another example, the changed variable can be the form in which the SAM components are introduced. For example, the guide RNA can be introduced in the form of DNA or in the form of RNA, and the chimeric Cas protein and chimeric adaptor protein can be introduced in the form of DNA, RNA, or protein. Similarly, the guide RNA or chimeric Cas protein or chimeric adaptor protein (or nucleic acids encoding such components) can comprise various combinations of modifications for stability, to reduce off-target effects, to facilitate delivery, and so forth. As another example, the changed variable can be the sequence of the guide RNA that is introduced (e.g., introducing a different guide RNA with a different sequence or targeting a different guide RNA target sequence).

Methods are also provided for using the eukaryotic cells or organisms generated by the methods disclosed herein for increasing or activating expression or transcription of a target gene, particularly for target genes whose overexpression is associated with or causative of a disease. Such eukaryotic cells or organisms having increased expression of a target gene whose overexpression is associated with or causative of a disease can be used, for example, to screen compounds for therapeutic or prophylactic effect against the disease or for efficacy in decreasing expression of the target gene. Such methods can comprise, for example, increasing or activating transcription of the target gene in a eukaryotic cell or organism as described elsewhere herein, introducing into the eukaryotic cell or organism a reagent or compound, and then assessing activity of the reagent or compound (e.g., in a eukaryotic cell or organism treated with the reagent or compound compared to a control eukaryotic cell or organism not treated with the reagent or compound). The assessing can comprise, for example, assessing expression of the target gene (e.g., at the mRNA level or at the protein level), wherein a decrease in expression of the target gene can indicate a therapeutic or prophylactic effect. Alternatively or additionally, the assessing can comprise assessing one of more signs or symptoms of the disease associated with or caused by overexpression of the target gene, wherein a decrease in the presence of or amelioration of a sign or symptom can indicate a therapeutic or prophylactic effect. A screened reagent or compound can then be selected as a candidate therapeutic or prophylactic reagent or compound if it shows a therapeutic or prophylactic effect.

Methods are also provided for increasing or activating expression or transcription of a target gene in a subject in need thereof, wherein decreased expression or activity of the target gene is associated with or causative of a disease, disorder, or syndrome. For example, such methods can be for increasing or activating expression or transcription of a target gene, particularly for target genes whose underexpression is associated with or causative of a disease or condition, or is associated with or causative of susceptibility to a disease or condition or side effects of a medication. For example, the target gene can be one that is underexpressed or expressed at low levels in the subject, and the underexpression or low level of expression is associated with or causative of a disease, disorder, or syndrome. Reduced transcription of such target genes, reduced amount of the gene products from such target genes, or reduced activity of the gene products from such target genes can be associated with, can exacerbate, or can cause a disease such that increasing transcription or expression of the target gene would be beneficial. One example of such a gene is OTC (Entrez Gene ID 5009). Other examples of such genes are HBG1 (Entrez Gene ID 3047) and HBG2 (Entrez Gene ID 3048). Other examples of such genes include haploinsufficient genes such as those in Tables 2 and 3. The subject can be, for example, a subject with decreased expression or activity of the target gene, such as a subject with a disease, disorder, or syndrome associated with haploinsufficiency.

### III. CRISPR/Cas Synergistic Activation Mediator (SAM) Systems

The methods and compositions (e.g. lipid nanoparticles) disclosed herein utilize Clustered Regularly Interspersed Short Palindromic Repeats (CRISPR)/CRISPR-associated (Cas)-based synergistic activation mediator (SAM) systems for use in methods of activating transcription of target genes in vivo or ex vivo and to assess the ability of SAM systems or components of such systems (e.g., guide RNAs) to activate transcription of a target genomic locus in vivo or ex vivo. The SAM system components described herein are delivered all together in the same lipid nanoparticle and comprise chimeric Cas proteins, chimeric adaptor proteins, and guide RNAs as described elsewhere herein to activate transcription of target genes. Chimeric Cas proteins (e.g., chimeric Cas proteins, such as chimeric Cas9 proteins, such as a chimeric Streptococcus pyogenes Cas9 protein, a chimeric Campylobacter jejuni Cas9 protein, or a chimeric Staphylococcus aureus Cas9 protein (e.g., a chimeric Cas9 protein derived from a Streptococcus pyogenes Cas9 protein, a Campylobacter jejuni Cas9 protein, or a Staphylococcus aureus Cas9 protein) and chimeric adaptor proteins (e.g., comprising an adaptor protein that specifically binds to an adaptor-binding element within a guide RNA and one or more heterologous transcriptional activation domains) are described in further detail elsewhere herein. In one example, the chimeric Cas protein and the chimeric adaptor protein are delivered in a single multicistronic or bicistronic nucleic acid (e.g., DNA or mRNA) (referred to as SAM cassette or SAM mRNA). For example, the sequence encoding the chimeric Cas protein and the sequence encoding the chimeric adaptor protein can be linked by a sequence encoding a 2A protein as described in more detail elsewhere herein. In a specific example, the chimeric Cas protein (e.g., NLS-Cas9-NLS-VP64 in which, for example, the 5′ NLS is monopartite and the 3′ NLS is bipartite) can be provided as a multicistronic or bicistronic mRNA (e.g., in vitro transcribed mRNA) that also encodes a chimeric adaptor protein (e.g., MS2(MCP)-NLS-p65-HSF1). The nucleic acids encoding the chimeric Cas protein and the chimeric adaptor protein can be linked by a nucleic acid encoding a 2A protein. As one example, the mRNA can comprise from 5′ to 3′: NLS-Cas9-NLS-VP64-2A-MS2(MCP)-NLS-p65-HSF1. The mRNA can be capped at the 5′ end (e.g., a cap 1 structure in which the +1 ribonucleotide is methylated at the 2′O position of the ribose), can be polyadenylated (poly(A) tail), and can optionally also be modified to be fully substituted with pseudouridine.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). CRISPR/Cas systems used in the compositions and methods disclosed herein can be non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

The methods and compositions disclosed herein employ the CRISPR/Cas systems by using or testing the ability of CRISPR complexes (comprising a guide RNA (gRNA) complexed with a chimeric Cas protein and a chimeric adaptor protein) to induce transcriptional activation of a target genomic locus in vivo.

A. Chimeric Cas Proteins

Provided are chimeric Cas proteins that can bind to the guide RNAs disclosed elsewhere herein to activate transcription of target genes. Such chimeric Cas proteins can comprise: (a) a DNA-binding domain that is a Clustered Regularly Interspaced Short Palindromic Repeats (CRISPR)-associated (Cas) protein or a functional fragment or variant thereof that is capable of forming a complex with a guide RNA and binding to a target sequence; and (b) one or more transcriptional activation domains or functional fragments or variants thereof. For example, such fusion proteins can comprise 1, 2, 3, 4, 5, or more transcriptional activation domains (e.g., two or more heterologous transcriptional activation domains or three or more heterologous transcriptional activation domains). In one example, the chimeric Cas protein can comprise a catalytically inactive Cas protein (e.g., dCas9) and a VP64 transcriptional activation domain or a functional fragment or variant thereof. For example, such a chimeric Cas protein can comprise, consist essentially of, or consist of an amino acid sequence at least 85%, 90%, 91%, 92%, 93%, 94%, 95%, 96%, 97%, 98%, 99%, or 100% identical to the dCas9-VP64 chimeric Cas protein sequence set forth in SEQ ID NO: 1. However, chimeric Cas proteins in which the transcriptional activation domains comprise other transcriptional activation domains or functional fragments or variants thereof and/or in which the Cas protein comprises other Cas proteins (e.g., catalytically inactive Cas proteins) are also provided. Examples of other suitable transcriptional activation domains are provided elsewhere herein.

The transcriptional activation domain(s) can be located at the N-terminus, the C-terminus, or anywhere within the Cas protein. For example, the transcriptional activation domain(s) can be attached to the Rec1 domain, the Rec2 domain, the HNH domain, or the PI domain of a Streptococcus pyogenes Cas9 protein or any corresponding region of an orthologous Cas9 protein or homologous or orthologous Cas protein when optimally aligned with the S. pyogenes Cas9 protein. For example, the transcriptional activation domain can be attached to the Rec1 domain at position 553, the Rec1 domain at position 575, the Rec2 domain at any position within positions 175-306 or replacing part of or the entire region within positions 175-306, the HNH domain at any position within positions 715-901 or replacing part of or the entire region within positions 715-901, or the PI domain at position 1153 of the S. pyogenes Cas9 protein. See, e.g., WO 2016/049258, herein incorporated by reference in its entirety for all purposes. The transcriptional activation domain may be flanked by one or more linkers on one or both sides as described elsewhere herein.

Chimeric Cas proteins can also be operably linked or fused to additional heterologous polypeptides. The fused or linked heterologous polypeptide can be located at the N-terminus, the C-terminus, or anywhere internally within the chimeric Cas protein. For example, a chimeric Cas protein can further comprise a nuclear localization signal. Examples of suitable nuclear localization signals and other modifications to Cas proteins are described in further detail elsewhere herein.

Chimeric Cas proteins can be provided in any form. For example, a chimeric Cas protein can be provided in the form of a protein, such as a chimeric Cas protein complexed with a gRNA. Alternatively, a chimeric Cas protein can be provided in the form of a nucleic acid encoding the chimeric Cas protein, such as an RNA (e.g., messenger RNA (mRNA)) or DNA. In a specific example, the chimeric Cas protein can be provided as a mRNA (e.g., in vitro transcribed mRNA), such as a multicistronic or bicistronic mRNA that also encodes a chimeric adaptor protein. Optionally, the nucleic acid encoding the chimeric Cas protein can be codon-optimized for efficient translation into protein in a particular cell or organism. For example, the nucleic acid encoding the chimeric Cas protein can be modified to substitute codons having a higher frequency of usage in a eukaryotic cell, a non-human eukaryotic cell, an animal cell, a non-human animal cell, a mammalian cell, a non-human mammalian cell, a human cell, a non-human cell, a rodent cell, a mouse cell, a rat cell, or any other host cell of interest, as compared to the naturally occurring polynucleotide sequence. When a nucleic acid encoding the chimeric Cas protein is introduced into the cell, the chimeric Cas protein can be transiently, conditionally, or constitutively expressed in the cell.

Chimeric Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding chimeric Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding chimeric Cas proteins can also be polyadenylated (to comprise a poly(A) tail). mRNA encoding chimeric Cas proteins can also be modified to include pseudouridine (e.g., can be fully substituted with pseudouridine). For example, capped and polyadenylated chimeric Cas mRNA containing N1-methyl pseudouridine can be used. Likewise, chimeric Cas mRNAs can be modified by depletion of uridine using synonymous codons. Other possible modifications are described in more detail elsewhere herein.

Chimeric Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding chimeric Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding chimeric Cas proteins can also be polyadenylated (to comprise a poly(A) tail). mRNA encoding chimeric Cas proteins can also be modified to include pseudouridine (e.g., can be fully substituted with pseudouridine). For example, capped and polyadenylated chimeric Cas mRNA containing N1-methyl pseudouridine can be used. Likewise, chimeric Cas mRNAs can be modified by depletion of uridine using synonymous codons.

Chimeric Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Chimeric Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes. CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Chimeric Cas mRNAs can further comprise a poly-adenylated (poly-A) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

Nucleic acids encoding chimeric Cas proteins can be for stable integration into the genome of a cell and operably linking to a promoter active in the cell. Alternatively, nucleic acids encoding chimeric Cas proteins can be operably linked to a promoter in an expression construct. Expression constructs include any nucleic acid constructs capable of directing expression of a gene or other nucleic acid sequence of interest (e.g., a chimeric Cas gene) and which can transfer such a nucleic acid sequence of interest to a target cell. For example, the nucleic acid encoding the chimeric Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a non-human eukaryotic cell, an animal cell, a non-human animal cell, a mammalian cell, a non-human mammalian cell, a human cell, a non-human cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a chimeric Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a chimeric Cas protein and a guide RNA simultaneously allow for the generation of compact expression cassettes to facilitate delivery.

(1) Cas Proteins

Cas proteins generally comprise at least one RNA recognition or binding domain that can interact with guide RNAs. A functional fragment or functional variant of a Cas protein is one that retains the ability to form a complex with a guide RNA and to bind to a target sequence in a target gene (and, for example, activate transcription of the target gene).

In addition to transcriptional activation domain as described elsewhere herein, Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus. In one example, the Cas protein portions of the chimeric Cas proteins disclosed herein have been modified to have decreased nuclease activity (e.g., nuclease activity is diminished by at least about 70%, 75%, 80%, 85%, 90%, 95%, 96%, 97%, 98%, 99%, or 100% compared to a wild type Cas protein) or to lack substantially all nuclease activity (i.e., nuclease activity is diminished by at least 90%, 95%, 97%, 98%, 99%, or 100% compared to a wild type Cas protein, or having no more than about 0%, 1%, 2%, 3%, 5%, or 10% of the nuclease activity of a wild type Cas protein). A nuclease-inactive Cas protein is a Cas protein having mutations known to be inactivating mutations in its catalytic (i.e., nuclease) domains (e.g., inactivating mutations in a RuvC-like endonuclease domain in a Cpf1 protein, or inactivating mutations in both an HNH endonuclease domain and a RuvC-like endonuclease domain in Cas9) or a Cas protein having nuclease activity diminished by at least about 97%, 98%, 99%, or 100% compared to a wild type Cas protein. Examples of different Cas protein mutations to reduce or substantially eliminate nuclease activity are disclosed below.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (assigned SwissProt accession number Q99ZW2) is an exemplary Cas9 protein. Cas9 from S. aureus (SaCas9) (assigned UniProt accession number J7RUA5) is another exemplary Cas9 protein. Cas9 from Campylobacter jejuni (CjCas9) (assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, and WO 2019/067910, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9. One example of a catalytically inactive Cas9 protein (dCas9) comprises, consists essentially of, or consist of an amino acid sequence at least 85%, 90%, 91%, 92%, 93%, 94%, 95%, 96%, 97%, 98%, 99%, or 100% identical to the dCas9 protein sequence set forth in SEQ ID NO: 2.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) and a substitution at position D10 (e.g., D10A substitution) to generate a nuclease-inactive Cas protein. See, e.g., WO 2016/106236, herein incorporated by reference in its entirety for all purposes. Examples of inactivating mutations in the catalytic domains of Nme2Cas9 are also known (e.g., combination of D16A and H588A). Examples of inactivating mutations in the catalytic domains of St1Cas9 are also known (e.g., combination of D9A, D598A, H599A, and N622A). Examples of inactivating mutations in the catalytic domains of St3Cas9 are also known (e.g., combination of D10A and N870A). Examples of inactivating mutations in the catalytic domains of CjCas9 are also known (e.g., combination of D8A and H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Cas proteins can also be operably linked to heterologous polypeptides as fusion proteins. For example, in addition to transcriptional activation domains, a Cas protein can be fused to a cleavage domain or an epigenetic modification domain. See WO 2014/089290, herein incorporated by reference in its entirety for all purposes. Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

In one example, a Cas protein may be fused with 1-10 NLSs, 1-5 NLSs, or one NLS. Where one NLS is used, the NLS may be linked at the N-terminus or the C-terminus of the Cas sequence. It may also be inserted internally within the Cas sequence. In other examples, the Cas protein may be fused with more than one NLS. For example, the Cas protein may be fused with 2, 3, 4, or 5 NLSs or may fused with two NLSs. In certain circumstances, the two NLSs may be the same (e.g., two SV40 NLSs) or different. For example, the Cas protein may be fused to two SV40 NLS sequences linked at the carboxy terminus. In another example, the Cas protein may be fused with two NLSs, one linked at the N-terminus and one at the C-terminus. In another example, the Cas protein may be fused with 3 NLSs. In another example, the Cas protein may be fused with no NLS. In some examples, the NLS may be a monopartite sequence, such as, for example, the SV40 NLS, PKKKRKV (SEQ ID NO: 58) or PKKKRRV (SEQ ID NO: 59). In some examples, the NLS may be a bipartite sequence, such as the NLS of nucleoplasmin, KRPAATKKAGQAKKKK (SEQ ID NO: 60). In a specific example, a single PKKKRKV (SEQ ID NO: 58) NLS may be linked at the C-terminus of the RNA-guided DNA-binding agent. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification) or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

(2) Transcriptional Activation Domains

The chimeric Cas proteins disclosed herein can comprise one or more transcriptional activation domains. Transcriptional activation domains include regions of a naturally occurring transcription factor which, in conjunction with a DNA-binding domain (e.g., a catalytically inactive Cas protein complexed with a guide RNA), can activate transcription from a promoter by contacting transcriptional machinery either directly or through other proteins such as coactivators. Transcriptional activation domains also include functional fragments or variants of such regions of a transcription factor and engineered transcriptional activation domains that are derived from a native, naturally occurring transcriptional activation domain or that are artificially created or synthesized to activate transcription of a target gene. A functional fragment is a fragment that is capable of activating transcription of a target gene when operably linked to a suitable DNA-binding domain. A functional variant is a variant that is capable of activating transcription of a target gene when operably linked to a suitable DNA-binding domain.

A specific transcriptional activation domain for use in the chimeric Cas proteins disclosed herein comprises a VP64 transcriptional activation domain or a functional fragment or variant thereof. VP64 is a tetrameric repeat of the minimal activation domain from the herpes simplex VP16 activation domain. For example, the transcriptional activation domain can comprise, consist essentially of, or consist of an amino acid sequence at least 85%, 90%, 91%, 92%, 93%, 94%, 95%, 96%, 97%, 98%, 99%, or 100% identical to the VP64 transcriptional activation domain protein sequence set forth in SEQ ID NO: 3.

Other examples of transcriptional activation domains include herpes simplex virus VP16 transactivation domain, VP64 (quadruple tandem repeat of the herpes simplex virus VP16), a NF-κB p65 (NF-κB trans-activating subunit p65) activation domain, a MyoD1 transactivation domain, an HSF1 transactivation domain (transactivation domain from human heat-shock factor 1), RTA (Epstein Barr virus R transactivator activation domain), a SETT/9 transactivation domain, a p53 activation domain 1, a p53 activation domain 2, a CREB (cAMP response element binding protein) activation domain, an E2A activation domain, an NFAT (nuclear factor of activated T-cells) activation domain, and functional fragments and variants thereof. See, e.g., US 2016/0298125, US 2016/0281072, and WO 2016/049258, each of which is herein incorporated by reference in its entirety for all purposes. Other examples of transcriptional activation domains include Gcn4, MLL, Rtg3, Gln3, Oaf1, Pip2, Pdr1, Pdr3, Pho4, Leu3, and functional fragments and variants thereof. See, e.g., US 2016/0298125, herein incorporated by reference in its entirety for all purposes. Yet other examples of transcriptional activation domains include Sp1, Vax, GATA4, and functional fragments and variants thereof. See, e.g., WO 2016/149484, herein incorporated by reference in its entirety for all purposes. Other examples include activation domains from Oct1, Oct-2A, AP-2, CTF1, P300, CBP, PCAF, SRC1, PvALF, ERF-2, OsGAI, HALF-1, C1, AP1, ARF-5, ARF-6, ARF-7, ARF-8, CPRF1, CPRF4, MYC-RP/GP, and TRAB1PC4, and functional fragments and variants thereof. See, e.g., US 2016/0237456, EP3045537, and WO 2011/146121, each of which is incorporated by reference in its entirety for all purposes. Additional suitable transcriptional activation domains are also known. See, e.g., WO 2011/146121, herein incorporated by reference in its entirety for all purposes.

B. Chimeric Adaptor Proteins

Also provided are chimeric adaptor proteins that can bind to the guide RNAs disclosed elsewhere herein. The chimeric adaptor proteins disclosed herein are useful in dCas-synergistic activation mediator (SAM)-like systems to increase the number and diversity of transcriptional activation domains being directed to a target sequence within a target gene to activate transcription of the target gene.

Such chimeric adaptor proteins comprise: (a) an adaptor (i.e., adaptor domain or adaptor protein) that specifically binds to an adaptor-binding element within a guide RNA; and (b) one or more heterologous transcriptional activation domains. For example, such fusion proteins can comprise 1, 2, 3, 4, 5, or more transcriptional activation domains (e.g., two or more heterologous transcriptional activation domains or three or more heterologous transcriptional activation domains). In one example, such chimeric adaptor proteins can comprise: (a) an adaptor (i.e., an adaptor domain or adaptor protein) that specifically binds to an adaptor-binding element in a guide RNA; and (b) two or more transcriptional activation domains. For example, the chimeric adaptor protein can comprise: (a) an MS2 coat protein adaptor that specifically binds to one or more MS2 aptamers in a guide RNA (e.g., two MS2 aptamers in separate locations in a guide RNA); and (b) one or more (e.g., two or more transcriptional activation domains). For example, the two transcriptional activation domains can be p65 and HSF1 transcriptional activation domains or functional fragments or variants thereof. However, chimeric adaptor proteins in which the transcriptional activation domains comprise other transcriptional activation domains or functional fragments or variants thereof are also provided.

The one or more transcriptional activation domains can be fused directly to the adaptor. Alternatively, the one or more transcriptional activation domains can be linked to the adaptor via a linker or a combination of linkers or via one or more additional domains. Likewise, if two or more transcriptional activation domains are present, they can be fused directly to each other or can be linked to each other via a linker or a combination of linkers or via one or more additional domains. Linkers that can be used in these fusion proteins can include any sequence that does not interfere with the function of the fusion proteins. Exemplary linkers are short (e.g., 2-20 amino acids) and are typically flexible (e.g., comprising amino acids with a high degree of freedom such as glycine, alanine, and serine). Some specific examples of linkers comprise one or more units consisting of GGGS (SEQ ID NO: 4) or GGGGS (SEQ ID NO: 5), such as two, three, four, or more repeats of GGGS (SEQ ID NO: 4) or GGGGS (SEQ ID NO: 5) in any combination. Other linker sequences can also be used.

The one or more transcriptional activation domains and the adaptor can be in any order within the chimeric adaptor protein. As one option, the one or more transcriptional activation domains can be C-terminal to the adaptor and the adaptor can be N-terminal to the one or more transcriptional activation domains. For example, the one or more transcriptional activation domains can be at the C-terminus of the chimeric adaptor protein, and the adaptor can be at the N-terminus of the chimeric adaptor protein. However, the one or more transcriptional activation domains can be C-terminal to the adaptor without being at the C-terminus of the chimeric adaptor protein (e.g., if a nuclear localization signal is at the C-terminus of the chimeric adaptor protein). Likewise, the adaptor can be N-terminal to the one or more transcriptional activation domains without being at the N-terminus of the chimeric adaptor protein (e.g., if a nuclear localization signal is at the N-terminus of the chimeric adaptor protein). As another option, the one or more transcriptional activation domains can be N-terminal to the adaptor and the adaptor can be C-terminal to the one or more transcriptional activation domains. For example, the one or more transcriptional activation domains can be at the N-terminus of the chimeric adaptor protein, and the adaptor can be at the C-terminus of the chimeric adaptor protein. As yet another option, if the chimeric adaptor protein comprises two or more transcriptional activation domains, the two or more transcriptional activation domains can flank the adaptor.

Chimeric adaptor proteins can also be operably linked or fused to additional heterologous polypeptides. The fused or linked heterologous polypeptide can be located at the N-terminus, the C-terminus, or anywhere internally within the chimeric adaptor protein. For example, a chimeric adaptor protein can further comprise a nuclear localization signal. A specific example of such a protein comprises an MS2 coat protein (adaptor) linked (either directly or via an NLS) to a p65 transcriptional activation domain C-terminal to the MS2 coat protein (MCP), and HSF1 transcriptional activation domain C-terminal to the p65 transcriptional activation domain. Such a protein can comprise from N-terminus to C-terminus: an MCP; a nuclear localization signal; a p65 transcriptional activation domain; and an HSF1 transcriptional activation domain. For example, a chimeric adaptor protein can comprise, consist essentially of, or consist of an amino acid sequence at least 85%, 90%, 91%, 92%, 93%, 94%, 95%, 96%, 97%, 98%, 99%, or 100% identical to the MCP-p65-HSF1 chimeric adaptor protein sequence set forth in SEQ ID NO: 6.

Chimeric adaptor proteins can also be fused or linked to one or more heterologous polypeptides that provide for subcellular localization. Such heterologous polypeptides can include, for example, one or more nuclear localization signals (NLS) such as the SV40 NLS and/or an alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the chimeric adaptor protein (e.g., at the C-terminus or N-terminus of the adaptor protein component of the chimeric adaptor protein or at the C-terminus or N-terminus of a transcriptional activator domain component of the chimeric adaptor protein). An NLS can comprise, for example, a stretch of basic amino acids, and can be a monopartite sequence or a bipartite sequence. Optionally, the chimeric adaptor protein comprises two or more NLSs, including an NLS (e.g., an alpha-importin NLS) at the N-terminus and/or an NLS (e.g., an SV40 NLS) at the C-terminus. A chimeric adaptor protein can also comprise two or more NLSs at the N-terminus and/or two or more NLSs at the C-terminus.

In one example, a chimeric adaptor protein may be fused with 1-10 NLSs, 1-5 NLSs, or one NLS. Where one NLS is used, the NLS may be linked at the N-terminus or the C-terminus of the chimeric adaptor protein sequence. It may also be inserted internally within the chimeric adaptor protein sequence. In other examples, the chimeric adaptor protein may be fused with more than one NLS. For example, the chimeric adaptor protein may be fused with 2, 3, 4, or 5 NLSs or may fused with two NLSs. In certain circumstances, the two NLSs may be the same (e.g., two SV40 NLSs) or different. For example, the chimeric adaptor protein may be fused to two SV40 NLS sequences linked at the carboxy terminus. In another example, the chimeric adaptor protein may be fused with two NLSs, one linked at the N-terminus and one at the C-terminus. In another example, the chimeric adaptor protein may be fused with 3 NLSs. In another example, the chimeric adaptor protein may be fused with no NLS. In some examples, the NLS may be a monopartite sequence, such as, for example, the SV40 NLS, PKKKRKV (SEQ ID NO: 58) or PKKKRRV (SEQ ID NO: 59). In some examples, the NLS may be a bipartite sequence, such as the NLS of nucleoplasmin, KRPAATKKAGQAKKKK (SEQ ID NO: 60). In a specific example, a single PKKKRKV (SEQ ID NO: 58) NLS may be linked at the C-terminus of the RNA-guided DNA-binding agent. One or more linkers are optionally included at the fusion site.

Chimeric adaptor proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO2013/176772, each of which is herein incorporated by reference in its entirety for all purposes. As another example, chimeric adaptor proteins can be fused or linked to a heterologous polypeptide providing increased or decreased stability.

Chimeric adaptor proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Chimeric adaptor proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification) or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the chimeric adaptor protein. Likewise, the chimeric adaptor protein can be tethered to the 5′ end, the 3′ end, or to an internal region within the labeled nucleic acid. That is, the labeled nucleic acid can be tethered in any orientation and polarity.

Chimeric adaptor proteins can be provided in any form. For example, a chimeric adaptor protein can be provided in the form of a protein, such as a chimeric adaptor protein complexed with a gRNA. Alternatively, a chimeric adaptor protein can be provided in the form of a nucleic acid encoding the chimeric adaptor protein, such as an RNA (e.g., messenger RNA (mRNA)) or DNA. In a specific example, the chimeric adaptor protein can be provided as a mRNA (e.g., in vitro transcribed mRNA), such as a multicistronic or bicistronic mRNA that also encodes a chimeric Cas protein. Optionally, the nucleic acid encoding the chimeric adaptor protein can be codon-optimized for efficient translation into protein in a particular cell or organism. For example, the nucleic acid encoding the chimeric adaptor protein can be modified to substitute codons having a higher frequency of usage in a eukaryotic cell, a non-human eukaryotic cell, an animal cell, a non-human animal cell, a mammalian cell, a non-human mammalian cell, a human cell, a non-human cell, a rodent cell, a mouse cell, a rat cell, or any other host cell of interest, as compared to the naturally occurring polynucleotide sequence. When a nucleic acid encoding the chimeric adaptor protein is introduced into the cell, the chimeric adaptor protein can be transiently, conditionally, or constitutively expressed in the cell.

Chimeric adaptor proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding chimeric adaptor proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding chimeric adaptor proteins can also be polyadenylated (to comprise a poly(A) tail). mRNA encoding chimeric adaptor proteins can also be modified to include pseudouridine (e.g., can be fully substituted with pseudouridine). For example, capped and polyadenylated chimeric adaptor mRNA containing N1-methyl pseudouridine can be used. Likewise, chimeric adaptor mRNAs can be modified by depletion of uridine using synonymous codons. Other possible modifications are described in more detail elsewhere herein.

Chimeric adaptor proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding chimeric adaptor proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding chimeric adaptor proteins can also be polyadenylated (to comprise a poly(A) tail). mRNA encoding chimeric adaptor proteins can also be modified to include pseudouridine (e.g., can be fully substituted with pseudouridine). For example, capped and polyadenylated chimeric adaptor mRNA containing N1-methyl pseudouridine can be used. Likewise, chimeric adaptor mRNAs can be modified by depletion of uridine using synonymous codons.

Chimeric adaptor mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Chimeric adaptor mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes. CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Chimeric adaptor mRNAs can further comprise a poly-adenylated (poly-A) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

Nucleic acids encoding chimeric adaptor proteins can be stably integrated in the genome of a cell and operably linked to a promoter active in the cell. Alternatively, nucleic acids encoding chimeric adaptor proteins can be operably linked to a promoter in an expression construct. Expression constructs include any nucleic acid constructs capable of directing expression of a gene or other nucleic acid sequence of interest (e.g., a chimeric adaptor gene) and which can transfer such a nucleic acid sequence of interest to a target cell. For example, the nucleic acid encoding the chimeric adaptor protein can be in a vector comprising a DNA encoding a gRNA and/or a chimeric Cas protein. Alternatively, it can be in a vector or plasmid that is separate from the vector comprising the DNA encoding the gRNA or the DNA encoding the chimeric Cas protein. Promoters that can be used in an expression construct include promoters active, for example, in one or more of a eukaryotic cell, a non-human eukaryotic cell, an animal cell, a non-human animal cell, a mammalian cell, a non-human mammalian cell, a human cell, a non-human cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes.

(1) Adaptors

Adaptors (i.e., adaptor domains or adaptor proteins) are nucleic-acid-binding domains (e.g., DNA-binding domains and/or RNA-binding domains) that specifically recognize and bind to distinct sequences (e.g., bind to distinct DNA and/or RNA sequences such as aptamers in a sequence-specific manner). Aptamers include nucleic acids that, through their ability to adopt a specific three-dimensional conformation, can bind to a target molecule with high affinity and specificity. Such adaptors can bind, for example, to a specific RNA sequence and secondary structure. These sequences (i.e., adaptor-binding elements) can be engineered into a guide RNA. For example, an MS2 aptamer can be engineered into a guide RNA to specifically bind an MS2 coat protein (MCP). For example, the adaptor can comprise, consist essentially of, or consist of an amino acid sequence at least 85%, 90%, 91%, 92%, 93%, 94%, 95%, 96%, 97%, 98%, 99%, or 100% identical to the MCP sequence set forth in SEQ ID NO: 7.

Some specific examples of adaptors and targets include RNA-binding protein/aptamer combinations that exist within the diversity of bacteriophage coat proteins. For example, the following adaptor proteins or functional fragments or variants thereof can be used: MS2 coat protein (MCP), PP7, Qβ, F2, GA, fr, JP501, M12, R17, BZ13, JP34, JP500, KU1, M11, MX1, TW18, VK, SP, FI, ID2, NL95, TW19, AP205, φCb5, Φ Cb8r, Φ Cb12r, ΦCb23r, 7s, and PRR1. See, e.g., WO 2016/049258, herein incorporated by reference in its entirety for all purposes. A functional fragment or functional variant of an adaptor protein is one that retains the ability to bind to a specific adaptor-binding element (e.g., ability to bind to a specific adaptor-binding sequence in a sequence-specific manner). For example, a PP7 Pseudomonas bacteriophage coat protein variant can be used in which amino acids 68-69 are mutated to SG and amino acids 70-75 are deleted from the wild type protein. See, e.g., Wu et al. (2012) Biophys. J. 102(12):2936-2944 and Chao et al. (2007) Nat. Struct. Mol. Biol. 15(1):103-105, each of which is herein incorporated by reference in its entirety for all purposes. Likewise, an MCP variant may be used, such as a N55K mutant. See, e.g., Spingola and Peabody (1994) J. Biol. Chem. 269(12):9006-9010, herein incorporated by reference in its entirety for all purposes.

Other examples of adaptor proteins that can be used include all or part of (e.g., the DNA-binding from) endoribonuclease Csy4 or the lambda N protein. See, e.g., U S 2016/0312198, herein incorporated by reference in its entirety for all purposes.

(2) Transcriptional Activation Domains

The chimeric adaptor proteins disclosed herein comprise one or more transcriptional activation domains. Such transcriptional activation domains can be naturally occurring transcriptional activation domains, can be functional fragments or functional variants of naturally occurring transcriptional activation domains, or can be engineered or synthetic transcriptional activation domains. Transcriptional activation domains that can be used include those described for use in chimeric Cas proteins elsewhere herein.

A specific transcriptional activation domain for use in the chimeric adaptor proteins disclosed herein comprises p65 and/or HSF1 transcriptional activation domains or functional fragments or variants thereof. The HSF1 transcriptional activation domain can be a transcriptional activation domain of human heat shock factor 1 (HSF1). The p65 transcriptional activation domain can be a transcriptional activation domain of transcription factor p65, also known as nuclear factor NF-kappa-B p65 subunit encoded by the RELA gene. As one example, a transcriptional activation domain can comprise, consist essentially of, or consist of an amino acid sequence at least 85%, 90%, 91%, 92%, 93%, 94%, 95%, 96%, 97%, 98%, 99%, or 100% identical to the p65 transcriptional activation domain protein sequence set forth in SEQ ID NO: 8. As another example, a transcriptional activation domain can comprise, consist essentially of, or consist of an amino acid sequence at least 85%, 90%, 91%, 92%, 93%, 94%, 95%, 96%, 97%, 98%, 99%, or 100% identical to the HSF1 transcriptional activation domain protein sequence set forth in SEQ ID NO: 9.

C. SAM Guide RNAs

Also provided are guide RNAs that can bind to the chimeric Cas proteins and chimeric adaptor proteins disclosed elsewhere herein to activate transcription of target genes.

One or more guide RNAs can be used in the methods or compositions disclosed herein. For example, two or more, three or more, four or more, or five or more guide RNAs can be used. Two or more of the guide RNAs can target a different target sequence in a single target gene. For example, two or more, three or more, four or more, or five or more guide RNAs can each target a different target sequence in a single target gene. Similarly, the guide RNAs can target multiple target genes (e.g., two or more, three or more, four or more, or five or more target genes). Examples of guide RNA target sequences are disclosed elsewhere herein.

(1) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a C5 gRNA is a S. pyogenes Cas9 gRNA or an equivalent thereof. In some of the methods and compositions disclosed herein, a C5 gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail, located downstream (3′) of the DNA-targeting segment, comprises, consists essentially of, or consists of GUUUUAGAGCUAUGCU (SEQ ID NO: 10). Any of the DNA-targeting segments disclosed herein can be joined to the 5′ end of SEQ ID NO: 10 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences comprise, consist essentially of, or consist of any one of AGCAUAGCAAGUUAAAAUAAGGCUAGUCCGUUAUCAACUUGAAAAAGUGGCACC GAGUCGGUGCUUU (SEQ ID NO: 11), AAACAGCAUAGCAAGUUAAAAUAAGGCUAGUCCGUUAUCAACUUGAAAAAGUGG CACCGAGUCGGUGCUUUU (SEQ ID NO: 50), or GUUGGAACCAUUCAAAACAGCAUAGCAAGUUAAAAUAAGGCUAGUCCGUUAUCA ACUUGAAAAAGUGGCACCGAGUCGGUGC (SEQ ID NO: 51).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, 15, 17, 18, 19, 20, 25, 30, 35, or 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, about 96%, about 97%, about 98%, about 99%, or about 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences comprise, consist essentially of, or consist of:

(version 1; SEQ ID NO: 12)
GUUUUAGAGCUAGAAAUAGCAAGUUAAAAUAAGGCUAGUCCGUUAUCAAC
UUGAAAAAGUGGCACCGAGUCGGUGCU;
(version 2; SEQ ID NO: 13)
GUUGGAACCAUUCAAAACAGCAUAGCAAGUUAAAAUAAGGCUAGUCCGUU
AUCAACUUGAAAAAGUGGCACCGAGUCGGUGC;
(version 3; SEQ ID NO: 14)
GUUUUAGAGCUAGAAAUAGCAAGUUAAAAUAAGGCUAGUCCGUUAUCAAC
UUGAAAAAGUGGCACCGAGUCGGUGC;
(version 4; SEQ ID NO: 15)
GUUUAAGAGCUAUGCUGGAAACAGCAUAGCAAGUUUAAAUAAGGCUAGUC
CGUUAUCAACUUGAAAAAGUGGCACCGAGUCGGUGC;
(version 5; SEQ ID NO: 52)
GUUUUAGAGCUAGAAAUAGCAAGUUAAAAUAAGGCUAGUCCGUUAUCAAC
UUGAAAAAGUGGCACCGAGUCGGUGCUUUUUUU;
(version 6; SEQ ID NO: 53)
GUUUUAGAGCUAGAAAUAGCAAGUUAAAAUAAGGCUAGUCCGUUAUCAAC
UUGAAAAAGUGGCACCGAGUCGGUGCUUUU;
or
(version 7; SEQ ID NO: 54)
GUUUAAGAGCUAUGCUGGAAACAGCAUAGCAAGUUUAAAUAAGGCUAGUC
CGUUAUCAACUUGAAAAAGUGGCACCGAGUCGGUGC.

Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, such as transcriptional activators); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Unmodified nucleic acids can be prone to degradation. Exogenous nucleic acids can also induce an innate immune response. Modifications can help introduce stability and reduce immunogenicity. Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification)); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

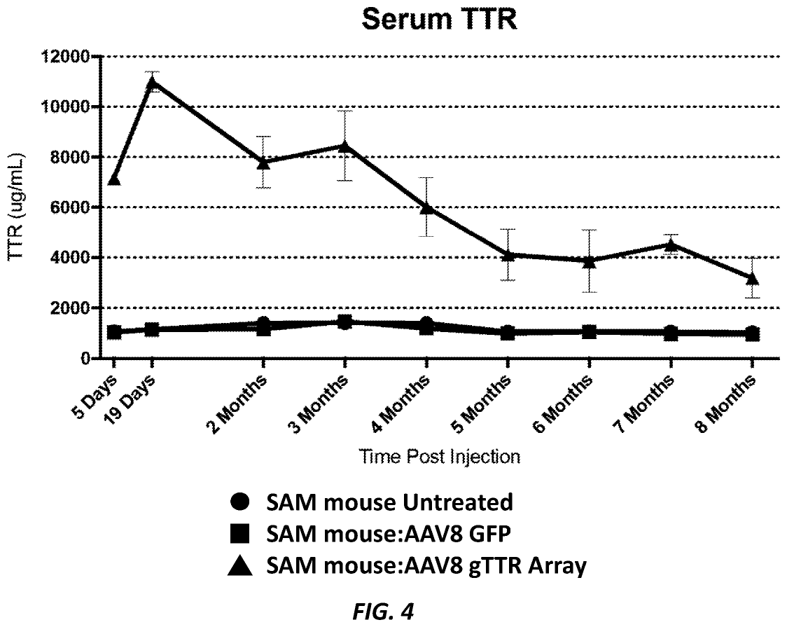

In some guide RNAs (e.g., single guide RNAs), at least one loop (e.g., two loops) of the guide RNA is modified by insertion of a distinct RNA sequence that binds to one or more adaptors (i.e., adaptor proteins or domains). Such adaptor proteins can be used to further recruit one or more heterologous functional domains, such as transcriptional activation domains. Examples of fusion proteins comprising such adaptor proteins (i.e., chimeric adaptor proteins) are disclosed elsewhere herein. For example, an MS2-binding loop ggccAACAUGAGGAUCACCCAUGUCUGCAGggcc (SEQ ID NO: 16) may replace nucleotides +13 to +16 and nucleotides +53 to +56 of the sgRNA scaffold (backbone) set forth in SEQ ID NO: 12, 14, 52, or 53 or the sgRNA backbone for the S. pyogenes CRISPR/Cas9 system described in WO 2016/049258 and Konermann et al. (2015) Nature 517(7536):583-588, each of which is herein incorporated by reference in its entirety for all purposes. See, e.g., FIG. 3. The guide RNA numbering used herein refers to the nucleotide numbering in the guide RNA scaffold sequence (i.e., the sequence downstream of the DNA-targeting segment of the guide RNA). For example, the first nucleotide of the guide RNA scaffold is +1, the second nucleotide of the scaffold is +2, and so forth. Residues corresponding with nucleotides +13 to +16 in SEQ ID NO: 12, 14, 52, or 53 are the loop sequence in the region spanning nucleotides +9 to +21 in SEQ ID NO: 12, 14, 52, or 53, a region referred to herein as the tetraloop. Residues corresponding with nucleotides +53 to +56 in SEQ ID NO: 12, 14, 52, or 53 are the loop sequence in the region spanning nucleotides +48 to +61 in SEQ ID NO: 12, 14, 52, or 53, a region referred to herein as the stem loop 2. Other stem loop sequences in in SEQ ID NO: 12, 14, 52, or 53 comprise stem loop 1 (nucleotides +33 to +41) and stem loop 3 (nucleotides +63 to +75). The resulting structure is an sgRNA scaffold in which each of the tetraloop and stem loop 2 sequences have been replaced by an MS2 binding loop. The tetraloop and stem loop 2 protrude from the Cas9 protein in such a way that adding an MS2-binding loop should not interfere with any Cas9 residues. Additionally, the proximity of the tetraloop and stem loop 2 sites to the DNA indicates that localization to these locations could result in a high degree of interaction between the DNA and any recruited protein, such as a transcriptional activator. Thus, in some sgRNAs, nucleotides corresponding to +13 to +16 and/or nucleotides corresponding to +53 to +56 of the guide RNA scaffold set forth in SEQ ID NO: 12, 14, 52, or 53 or corresponding residues when optimally aligned with any of these scaffold/backbones are replaced by the distinct RNA sequences capable of binding to one or more adaptor proteins or domains. Alternatively or additionally, adaptor-binding sequences can be added to the 5′ end or the 3′ end of a guide RNA. An exemplary guide RNA scaffold comprising MS2-binding loops in the tetraloop and stem loop 2 regions can comprise, consist essentially of, or consist of the sequence set forth in SEQ ID NO: 40 or 56. An exemplary generic single guide RNA comprising MS2-binding loops in the tetraloop and stem loop 2 regions can comprise, consist essentially of, or consist of the sequence set forth in SEQ ID NO: 45 or 57.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid. Promoters that can be used in such expression constructs include promoters active, for example, in one or more of a eukaryotic cell, a non-human eukaryotic cell, an animal cell, a non-human animal cell, a mammalian cell, a non-human mammalian cell, a human cell, a non-human cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

(2) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

It can be preferable for the target sequence to be adjacent to the transcription start site of a gene. For example, the target sequence can be within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair of the transcription start site, within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair upstream of the transcription start site, or within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair downstream of the transcription start site. Optionally, the target sequence is within the region 200 base pairs upstream of the transcription start site and 1 base pair downstream of the transcription start site (−200 to +1).

The target sequence can be within any gene desired to be targeted for transcriptional activation. In some cases, a target gene may be one that is a non-expressing gene or a weakly expressing gene (e.g., only minimally expressed above background, such as 1.1-fold, 1.2-fold, 1.3-fold, 1.4-fold, 1.5-fold, 1.6-fold, 1.7-fold, 1.8-fold, 1.9-fold, or 2-fold). The target gene may also be one that is expressed at low levels compared to a control gene. The target gene may also be one that is epigenetically silenced. The term “epigenetically silenced” refers to a gene that is not being transcribed or is being transcribed at a level that is decreased with respect to the level of transcription of the gene in a control sample (e.g., a corresponding control cell, such as a normal cell), due to a mechanism other than a genetic change such as a mutation. Epigenetic mechanisms of gene silencing are well known and include, for example, hypermethylation of CpG dinucleotides in a CpG island of the 5′ regulatory region of a gene and structural changes in chromatin due, for example, to histone acetylation, such that gene transcription is reduced or inhibited.

Target genes can include genes expressed in particular organs or tissues, such as the liver. Target genes can include disease-associated genes. A disease-associated gene refers to any gene that yields transcription or translation products at an abnormal level or in an abnormal form in cells derived from a disease-affected tissues compared with tissues or cells of a non-disease control. It may be a gene that becomes expressed at an abnormally high level, where the altered expression correlates with the occurrence and/or progression of the disease. A disease-associated gene also refers to a gene possessing a mutation or genetic variation that is responsible for the etiology of a disease. The transcribed or translated products may be known or unknown and may be at a normal or abnormal level. For example, target genes can be genes associated with protein aggregation diseases and disorders, such as Alzheimer's disease, Parkinson's disease, Huntington's disease, amyotrophic lateral sclerosis, prion diseases, and amyloidoses such as transthyretin amyloidosis (e.g., Ttr). Target genes can also be genes involved in pathways related to a disease or condition, such as hypercholesterolemia or atherosclerosis, or genes that when overexpressed can model such diseases or conditions. Target genes can also be genes expressed or overexpressed in one or more types of cancer. See, e.g., Santarius et al. (2010) Nat. Rev. Cancer 10(1):59-64, herein incorporated by reference in its entirety for all purposes.

One specific example of such a target gene is the Ttr gene. Optionally, the Ttr gene can comprise a pathogenic mutation (e.g., a mutation causing amyloidosis). Examples of such mutations are provided, e.g., in WO 2018/007871, herein incorporated by reference in its entirety for all purposes. An exemplary human TTR protein and an exemplary human TTR gene are identified by UniProt ID P02766 and Entrez Gene ID 7276, respectively. An exemplary mouse TTR protein and an exemplary mouse Ttr gene are identified by UniProt ID P07309 and Entrez Gene ID 22139, respectively. Transthyretin (TTR) is a protein found in the serum and cerebrospinal fluid that carries thyroid hormone and retinol-binding protein to retinol. The liver secretes TTR into the blood, while the choroid plexus secretes it into the cerebrospinal fluid. TTR is also produced in the retinal pigmented epithelium and secreted into the vitreous. Misfolded and aggregated TTR accumulates in multiple tissues and organs in the amyloid diseases senile systemic amyloidosis (SSA), familial amyloid polyneuropathy (FAP), and familial amyloid cardiomyopathy (FAC). Transthyretin (TTR) is a 127-amino acid, 55 kDa serum and cerebrospinal fluid transport protein primarily synthesized by the liver but also produced by the choroid plexus. It has also been referred to as prealbumin, thyroxine binding prealbumin, ATTR, TBPA, CTS, CTS1, HEL111, HsT2651, and PALB. In its native state, TTR exists as a tetramer. In homozygotes, homo-tetramers comprise identical 127-amino-acid beta-sheet-rich subunits. In heterozygotes, TTR tetramers can be made up of variant and/or wild-type subunits, typically combined in a statistical fashion. TTR is responsible for carrying thyroxine (T4) and retinol-bound RBP (retinol-binding protein) in both the serum and the cerebrospinal fluid. Examples of guide RNA target sequences (not including PAM) in the mouse Ttr gene are set forth in SEQ ID NOS: 34, 35, and 36, respectively. SEQ ID NO: 34 is located −63 of the Ttr transcription start site (genomic coordinates: build mm10, chr18, +strand, 20665187-20665209), SEQ ID NO: 35 is located −134 of the Ttr transcription start site (genomic coordinates: build mm10, chr18, +strand, 20665116-20665138), and SEQ ID NO: 36 is located −112 of the Ttr transcription start site (genomic coordinates: build mm10, chr18, +strand, 20665138-20665160). Guide RNA DNA-targeting segments corresponding to the guide RNA target sequences set forth in SEQ ID NOS: 34, 35, and 36, respectively, are set forth in SEQ ID NOS: 41, 42, and 43, respectively. Examples of single guide RNAs comprising these DNA-targeting segments are set forth in SEQ ID NOS: 37, 38, and 39 or 55, respectively.

Disease-associated genes can also include any gene for which increased production of the gene would be beneficial in a subject (e.g., for treating or preventing a disease). Such genes can be those whose underexpression or low expression is associated with or causative of a disease, disorder, or syndrome. For example, reduced transcription of such target genes, reduced amount of the gene products from such target genes, or reduced activity of the gene products from such target genes can be associated with, can exacerbate, or can cause a disease such that increasing transcription or expression of the target gene would be beneficial. One example of such a gene is OTC (Entrez Gene ID 5009). Other examples of such genes are HBG1 (Entrez Gene ID 3047) and HBG2 (Entrez Gene ID 3048). Other examples of such genes include haploinsufficient genes such as those in Tables 2 and 3.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N2 is any DNA nucleotide and is immediately 5′ of the sequence to which the DNA-targeting segment of the guide RNA hybridizes on the complementary strand of the target DNA. In some such cases, N₁ and N₂ can be complementary and the N₁—N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 17) or N₂₀NGG (SEQ ID NO: 18). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 19) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 17-19, including the 5′ G or GG and the 3′ GG or NGG. Yet other guide RNA target sequences plus PAMs can have between 14 and 20 nucleotides in length of SEQ ID NOS: 17-19.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

D. Nucleic Acids Encoding Chimeric Cas Protein, Chimeric Adaptor Protein, Guide RNA, or Synergistic Activation Mediator

The chimeric Cas protein, chimeric adaptor protein, and guide RNAs described in detail elsewhere herein can be provided in the form of nucleic acids (e.g., DNA or RNA) in the methods and compositions disclosed herein. For example, the nucleic acids can be chimeric Cas protein expression cassettes, chimeric adaptor protein expression cassettes, synergistic activation mediator (SAM) expression cassettes comprising nucleic acids encoding both a chimeric Cas protein and a chimeric adaptor protein, guide RNA expression cassettes, or any combination thereof. Such nucleic acids can be RNA (e.g., messenger RNA (mRNA)) or DNA, can be single-stranded or double-stranded, and can be linear or circular. For example, the nucleic acids can be chimeric Cas protein mRNAs, chimeric adaptor protein mRNAs, synergistic activation mediator (SAM) mRNAs comprising nucleic acids encoding both a chimeric Cas protein and a chimeric adaptor protein, guide RNAs, or any combination thereof. DNA can be part of a vector, such as an expression vector or a targeting vector. The vector can also be a viral vector such as adenoviral, adeno-associated viral, lentiviral, and retroviral vectors. When any of the nucleic acids disclosed herein is introduced into a cell, the encoded chimeric DNA-targeting protein, chimeric adaptor protein, or guide RNA can be transiently, conditionally, or constitutively expressed in the cell.

Optionally, the nucleic acids can be codon-optimized for efficient translation into protein in a particular cell or organism. For example, the nucleic acid can be modified to substitute codons having a higher frequency of usage in a eukaryotic cell, a non-human eukaryotic cell, an animal cell, a non-human animal cell, a mammalian cell, a non-human mammalian cell, a human cell, a non-human cell, a rodent cell, a mouse cell, a rat cell, or any other host cell of interest, as compared to the naturally occurring polynucleotide sequence.

In some compositions and methods, the Cas protein, chimeric adaptor protein, and guide RNAs can be provided in the form of RNA. Such RNAs may be modified RNAs. See, e.g., WO 2017/173054, US 2019/0136231, and WO 2018/107028, each of which is herein incorporated by reference in its entirety for all purposes. For example, one or more of the RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end (e.g., 5′ terminus or 3′ terminus). As one example, at least the first 1, 2, 3 or 4 nucleotides at the 5′ end can be modified, and at least the last 1, 2, 3, or 4 nucleotides at the 3′ end can be modified. For example, such modifications can include 2′-O-methyl modified nucleotides at the first 1, 2, 3, or 4 nucleotides at the 5′ end and/or 2′-O-methyl modified nucleotides at the last 1, 2, 3, or 4 nucleotides at the 3′ end. Additionally or alternatively, such modifications can include, for example, phosphorothioate linkages between one or more of the first four nucleotides at the 5′ end or between one or more of the last four nucleotides at the 3′ end. For example, the first four nucleotides at the 5′ end can be linked by phosphorothioate bonds, and/or the last four nucleotides at the 3′ end can be linked by phosphorothioate bonds. In a specific example, an RNA (e.g., a guide RNA, such as a chemically synthesized guide RNA) includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

Modified nucleosides or nucleotides can be present in a guide RNA or mRNA. A guide RNA or mRNA comprising one or more modified nucleosides or nucleotides is called a modified RNA to describe the presence of one or more non-naturally and/or naturally occurring components or configurations that are used instead of or in addition to the canonical A, G, C, and U residues. Modified nucleosides and nucleotides can include one or more of: (1) alteration or replacement of one or both of the non-linking phosphate oxygens and/or of one or more of the linking phosphate oxygens in the phosphodiester backbone linkage (backbone modification); (2) alteration or replacement of a constituent of the ribose sugar (e.g., of the 2′ hydroxyl on the ribose sugar) (sugar modification); (3) wholesale replacement of the phosphate moiety with dephospho linkers (backbone modification); (4) modification or replacement of a naturally occurring nucleobase (e.g., with a non-canonical nucleobase) (base modification); (5) replacement or modification of the ribose-phosphate backbone (backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (sugar modification).

The modifications can be combined to provide modified RNAs comprising nucleosides and nucleotides (residues) that can have two, three, four, or more modifications. For example, a modified residue can have a modified sugar and a modified nucleobase. In some examples, every base of a gRNA or mRNA is modified (e.g., all bases have a modified phosphate group, such as a phosphorothioate group). For example, all, or substantially all, of the phosphate groups of a gRNA or mRNA molecule can be replaced with phosphorothioate groups. In other examples, modified RNAs comprise at least one modified residue at or near the 5′ end of the RNA and/or at or near the 3′ end of the RNA.

In some examples, a modified gRNA or mRNA comprises one, two, three, or more modified residues. In some examples, the gRNA or mRNA comprises one, two, three, or more modified residues at each of the 5′ and the 3′ ends of the gRNA or mRNA. In some examples, a modified mRNA comprises 5, 10, 15, 50, 100, 200, 300, 400, 500, 600, 700, 800, 900, 1000, or more modified residues. In some examples, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified gRNA or mRNA are modified nucleosides or nucleotides.

Unmodified nucleic acids can be prone to degradation by, for example, cellular nucleases. For example, nucleases can hydrolyze nucleic acid phosphodiester bonds. To provide stability, RNAs described herein (e.g., guide RNAs or chimeric Cas protein mRNAs or chimeric adaptor protein mRNAs) can contain one or more modified nucleosides or nucleotides. In some examples, the modified RNA molecules described herein can exhibit a reduced innate immune response when introduced into a population of cells, both in vivo and ex vivo. The term innate immune response includes a cellular response to exogenous nucleic acids, including single stranded nucleic acids, which involves the induction of cytokine expression and release, particularly the interferons, and cell death.

In some examples of a backbone modification, the phosphate group of a modified residue can be modified by replacing one or more of the oxygens with a different substituent. Further, the modified residue (e.g., modified residue present in a modified nucleic acid) can include the wholesale replacement of an unmodified phosphate moiety with a modified phosphate group as described herein. In some examples, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates, and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration or the “S” configuration. The backbone can also be modified by replacement of a bridging oxygen (i.e., the oxygen that links the phosphate to the nucleoside) with nitrogen (bridged phosphoroamidates), sulfur (bridged phosphorothioates), and carbon (bridged methylenephosphonates). The replacement can occur at either linking oxygen or at both of the linking oxygens.

The phosphate group can be replaced by non-phosphorus containing connectors in certain backbone modifications. For example, the charged phosphate group can be replaced by a neutral moiety. Examples of moieties which can replace the phosphate group include, for example, methyl phosphonate, hydroxylamino, siloxane, carbonate, carboxy methyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. For example, the nucleobases can be tethered by a surrogate backbone. Examples include the morpholino, cyclobutyl, pyrrolidine and peptide nucleic acid (PNA) nucleoside surrogates.

The modified nucleosides and modified nucleotides can also include one or more modifications to the sugar group. For example, the 2′ hydroxyl group (OH) can be modified or replaced with a number of different oxy or deoxy substituents. Such modifications to the 2′ hydroxyl group can enhance the stability of the nucleic acid since the hydroxyl can no longer be deprotonated to form a 2′-alkoxide ion.

Examples of 2′ hydroxyl group modifications include alkoxy or aryloxy (OR, wherein R can be, e.g., alkyl, cycloalkyl, aryl, aralkyl, heteroaryl or a sugar) or polyethylene glycols (PEG), O(CH₂CH₂O)ₙCH₂CH₂OR where R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In one example, the 2′ hydroxyl group modification can be 2′-O-Me. In another example, the 2′ hydroxyl group modification can be a 2′-fluoro modification, which replaces the 2′ hydroxyl group with a fluoride. In other examples, the 2′ hydroxyl group modification can include locked nucleic acids (LNA) in which the 2′ hydroxyl can be connected, for example, by a C₁₋₆ alkylene or C₁₋₆ heteroalkylene bridge to the 4′ carbon of the same ribose sugar. Exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino), and aminoalkoxy, O(CH₂)ₙ-amino (wherein amino can be, e.g., NH₂, alkylamino, dialkylamino, heterocyclyl, arylamino, diarylamino, heteroarylamino, or diheteroarylamino, ethylenediamine, or polyamino). In some examples, the 2′ hydroxyl group modification can include unlocked nucleic acids (UNA) in which the ribose ring lacks the C2′-C3′ bond. In some examples, the 2′ hydroxyl group modification can include the methoxyethyl group (MOE), (OCH₂CH₂OCH₃, e.g., a PEG derivative).

Deoxy 2′ modifications can include hydrogen (i.e., deoxyribose sugars, e.g., at the overhang portions of partially dsRNA); halo (e.g., bromo, chloro, fluoro, or iodo); amino (wherein amino can be, e.g., —NH₂, alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl, and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing, for example, arabinose, as the sugar. The modified nucleic acids can also include abasic sugars. These abasic sugars can also be further modified at one or more of the constituent sugar atoms. The modified nucleic acids can also include one or more sugars that are in the L form (e.g., L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some examples, the nucleobase can include, for example, naturally-occurring and synthetic derivatives of a base.

One or more residues at one or both ends of the gRNA or mRNA may be chemically modified, or the entire gRNA or mRNA may be chemically modified. Some examples comprise a 5′ end modification. Some examples embodiments comprise a 3′ end modification. In certain gRNAs, one or more or all of the nucleotides in single stranded overhang of a gRNA molecule are deoxynucleotides. In certain modified mRNAs, the mRNA can contain 5′ end and/or 3′ end modifications.

Chimeric Cas proteins, chimeric adaptor proteins, or both can be provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding chimeric Cas proteins, mRNA encoding chimeric adaptor proteins, or SAM mRNAs encoding both can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding chimeric Cas proteins, mRNA encoding chimeric adaptor proteins, or SAM mRNAs encoding both can also be polyadenylated (to comprise a poly(A) tail). mRNA encoding chimeric Cas proteins, mRNAs encoding chimeric adaptor proteins, or SAM mRNAs encoding both can also be modified to include pseudouridine (e.g., can be fully substituted with pseudouridine). For example, capped and polyadenylated chimeric Cas mRNA, chimeric adaptor protein mRNA, or SAM mRNA containing N1-methyl pseudouridine can be used. Likewise, chimeric Cas mRNAs, chimeric adaptor protein mRNAs, or SAM mRNAs can be modified by depletion of uridine using synonymous codons. Other possible modifications are described in more detail elsewhere herein.

Chimeric Cas proteins and/or chimeric adaptor proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding chimeric Cas proteins, mRNA encoding chimeric adaptor proteins, or SAM mRNAs encoding both can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding chimeric Cas proteins, mRNA encoding chimeric adaptor proteins, or SAM mRNAs encoding both can also be polyadenylated (to comprise a poly(A) tail). mRNA encoding chimeric Cas proteins, mRNA encoding chimeric adaptor proteins, or SAM mRNAs encoding both can also be modified to include pseudouridine (e.g., can be fully substituted with pseudouridine). For example, capped and polyadenylated chimeric Cas mRNA, chimeric adaptor protein, or SAM mRNA containing N1-methyl pseudouridine can be used. Likewise, chimeric Cas mRNAs, chimeric adaptor protein mRNAs, or SAM mRNAs can be modified by depletion of uridine using synonymous codons.

Chimeric Cas mRNAs, chimeric adaptor mRNAs, or SAM mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Chimeric Cas mRNAs, chimeric adaptor protein mRNAs, or SAM mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Chimeric Cas mRNAs, chimeric adaptor protein mRNAs, or SAM mRNAs can further comprise a poly-adenylated (poly-A) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

Alternatively, the Cas protein, chimeric adaptor protein, and guide RNAs can be provided in the form of DNA. DNA or expression cassettes can be for stable integration into the genome (i.e., into a chromosome) of a cell or eukaryotic organism (e.g., animal, non-human animal, mammal, or non-human mammal) or it can be for expression outside of a chromosome (e.g., extrachromosomally replicating DNA). The stably integrated expression cassettes or nucleic acids can be randomly integrated into the genome of the eukaryotic organism (e.g., animal, non-human animal, mammal, or non-human mammal) (i.e., transgenic), or they can be integrated into a predetermined region of the genome of the eukaryotic organism (e.g., animal, non-human animal, mammal, or non-human mammal) (i.e., knock in).

A nucleic acid or expression cassette described herein can be operably linked to any suitable promoter for expression in vivo within a eukaryotic organism (e.g., animal, non-human animal, mammal, or non-human mammal) or ex vivo within a cell. The eukaryotic organism (e.g., animal, non-human animal, mammal, or non-human mammal) can be any suitable eukaryotic organism (e.g., animal, non-human animal, mammal, or non-human mammal) as described elsewhere herein. As one example, a nucleic acid or expression cassette (e.g., a chimeric Cas protein expression cassette, a chimeric adaptor protein expression cassette, or a SAM cassette comprising nucleic acids encoding both a chimeric Cas protein and a chimeric adaptor protein) can be for operably linking to an endogenous promoter at a genomic locus. Alternatively, cassette nucleic acid or expression cassette can be operably linked to an exogenous promoter, such as a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. Promoters that can be used in an expression construct include promoters active, for example, in one or more of a eukaryotic cell, a non-human eukaryotic cell, an animal cell, a non-human animal cell, a mammalian cell, a non-human mammalian cell, a human cell, a non-human cell, a rodent cell, a mouse cell, a rat cell, a hamster cell, a rabbit cell, a pluripotent cell, an embryonic stem (ES) cell, or a zygote. Such promoters can be, for example, conditional promoters, inducible promoters, constitutive promoters, or tissue-specific promoters.

For example, a nucleic acid encoding a guide RNA can be operably linked to a U6 promoter, such as a human U6 promoter or a mouse U6 promoter. Specific examples of suitable promoters (e.g., for expressing a guide RNA) include an RNA polymerase III promoter, such as a human U6 promoter, a rat U6 polymerase III promoter, or a mouse U6 polymerase III promoter.

Optionally, the promoter can be a bidirectional promoter driving expression of one gene (e.g., a gene encoding a chimeric Cas protein) and a second gene (e.g., a gene encoding a guide RNA or a chimeric adaptor protein) in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express two genes simultaneously allows for the generation of compact expression cassettes to facilitate delivery.

One or more of the nucleic acids can be together in a multicistronic expression construct or multicistronic messenger RNA. For example, a nucleic acid encoding a chimeric Cas protein and a nucleic acid encoding a chimeric adaptor protein can be together in a bicistronic expression construct. Multicistronic expression vectors simultaneously express two or more separate proteins from the same mRNA (i.e., a transcript produced from the same promoter). Suitable strategies for multicistronic expression of proteins include, for example, the use of a 2A peptide and the use of an internal ribosome entry site (IRES). For example, such constructs can comprise: (1) nucleic acids encoding one or more chimeric Cas proteins and one or more chimeric adaptor proteins; (2) nucleic acids encoding two or more chimeric adaptor proteins; (3) nucleic acids encoding two or more chimeric Cas proteins; (4) nucleic acids encoding two or more guide RNAs; (5) nucleic acids encoding one or more chimeric Cas proteins and one or more guide RNAs; (6) nucleic acids encoding one or more chimeric adaptor proteins and one or more guide RNAs; or (7) nucleic acids encoding one or more chimeric Cas proteins, one or more chimeric adaptor proteins, and one or more guide RNAs. As one example, such multicistronic vectors can use one or more internal ribosome entry sites (IRES) to allow for initiation of translation from an internal region of an mRNA. As another example, such multicistronic vectors can use one or more 2A peptides. These peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6(4): e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin. Biol. Ther. 5(5):627-638, herein incorporated by reference in its entirety for all purposes. Examples of 2A peptides that can be used include Thoseaasigna virus 2A (T2A); porcine teschovirus-1 2A (P2A); equine rhinitis A virus (ERAV) 2A (E2A); and FMDV 2A (F2A). Exemplary T2A, P2A, E2A, and F2A sequences include the following: T2A (EGRGSLLTCGDVEENPGP; SEQ ID NO: 20); P2A (ATNFSLLKQAGDVEENPGP; SEQ ID NO: 21); E2A (QCTNYALLKLAGDVESNPGP; SEQ ID NO: 22); and F2A (VKQTLNFDLLKLAGDVESNPGP; SEQ ID NO: 23). GSG residues can be added to the 5′ end of any of these peptides to improve cleavage efficiency.

Any of the nucleic acids or expression cassettes can also comprise a polyadenylation signal or transcription terminator upstream of a coding sequence. For example, a chimeric Cas protein expression cassette, a chimeric adaptor protein expression cassette, a SAM expression cassette, or a guide RNA expression cassette can comprise a polyadenylation signal or transcription terminator upstream of the coding sequence(s) in the expression cassette. The polyadenylation signal or transcription terminator can be flanked by recombinase recognition sites recognized by a site-specific recombinase. The polyadenylation signal or transcription terminator prevents transcription and expression of the protein or RNA encoded by the coding sequence (e.g., chimeric Cas protein, chimeric adaptor protein, guide RNA, or recombinase). However, upon exposure to the site-specific recombinase, the polyadenylation signal or transcription terminator will be excised, and the protein or RNA can be expressed.

Such a configuration for an expression cassette (e.g., a chimeric Cas protein expression cassette or a SAM expression cassette) can enable tissue-specific expression or developmental-stage-specific expression in eukaryotic organism (e.g., animal, non-human animal, mammal, or non-human mammal) comprising the expression cassette if the polyadenylation signal or transcription terminator is excised in a tissue-specific or developmental-stage-specific manner. For example, in the case of the chimeric Cas protein, this may reduce toxicity due to prolonged expression of the chimeric Cas protein in a cell or eukaryotic organism (e.g., animal, non-human animal, mammal, or non-human mammal) or expression of the chimeric Cas protein at undesired developmental stages or in undesired cell or tissue types within a eukaryotic organism (e.g., animal, non-human animal, mammal, or non-human mammal). See, e.g., Parikh et al. (2015) PLoS One 10(1):e0116484, herein incorporated by reference in its entirety for all purposes. Excision of the polyadenylation signal or transcription terminator in a tissue-specific or developmental-stage-specific manner can be achieved if a eukaryotic organism (e.g., animal, non-human animal, mammal, or non-human mammal) comprising the expression cassette further comprises a coding sequence for the site-specific recombinase operably linked to a tissue-specific or developmental-stage-specific promoter. The polyadenylation signal or transcription terminator will then be excised only in those tissues or at those developmental stages, enabling tissue-specific expression or developmental-stage-specific expression. In one example, a chimeric Cas protein, a chimeric adaptor protein, a chimeric Cas protein and a chimeric adaptor protein, or a guide RNA can be expressed in a liver-specific manner.

Any transcription terminator or polyadenylation signal can be used. A “transcription terminator” as used herein refers to a DNA sequence that causes termination of transcription. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The expression cassettes disclosed herein can comprise other components as well. Such expression cassettes (e.g., chimeric Cas protein expression cassette, chimeric adaptor protein expression cassette, SAM expression cassette, guide RNA expression cassette, or recombinase expression cassette) can further comprise a 3′ splicing sequence at the 5′ end of the expression cassette and/or a second polyadenylation signal following the coding sequence (e.g., encoding the chimeric Cas protein, the chimeric adaptor protein, or the guide RNA). The term 3′ splicing sequence refers to a nucleic acid sequence at a 3′ intron/exon boundary that can be recognized and bound by splicing machinery. An expression cassette can further comprise a selection cassette comprising, for example, the coding sequence for a drug resistance protein.

Examples of suitable selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), and herpes simplex virus thymidine kinase (HSV-k). Optionally, the selection cassette can be flanked by recombinase recognition sites for a site-specific recombinase. If the expression cassette also comprises recombinase recognition sites flanking a polyadenylation signal upstream of the coding sequence as described above, the selection cassette can be flanked by the same recombinase recognition sites or can be flanked by a different set of recombinase recognition sites recognized by a different recombinase.

An expression cassette can also comprise a nucleic acid encoding one or more reporter proteins, such as a fluorescent protein (e.g., a green fluorescent protein). Any suitable reporter protein can be used. For example, a fluorescent reporter protein can be used, or a non-fluorescent reporter protein can be used. Examples of fluorescent reporter proteins are provided elsewhere herein. Non-fluorescent reporter proteins include, for example, reporter proteins that can be used in histochemical or bioluminescent assays, such as beta-galactosidase, luciferase (e.g., Renilla luciferase, firefly luciferase, and NanoLuc luciferase), and beta-glucuronidase. An expression cassette can include a reporter protein that can be detected in a flow cytometry assay (e.g., a fluorescent reporter protein such as a green fluorescent protein) and/or a reporter protein that can be detected in a histochemical assay (e.g., beta-galactosidase protein). One example of such a histochemical assay is visualization of in situ beta-galactosidase expression histochemically through hydrolysis of X-Gal (5-bromo-4-chloro-3-indoyl-b-D-galactopyranoside), which yields a blue precipitate, or using fluorogenic substrates such as beta-methyl umbelliferyl galactoside (MUG) and fluorescein digalactoside (FDG).

The expression cassettes described herein can be in any form. For example, an expression cassette can be in a vector or plasmid. The expression cassette can be operably linked to a promoter in an expression construct capable of directing expression of a protein or RNA (e.g., upon removal of an upstream polyadenylation signal). Alternatively, an expression cassette can be in a targeting vector. For example, the targeting vector can comprise homology arms flanking the expression cassette, wherein the homology arms are suitable for directing recombination with a desired target genomic locus to facilitate genomic integration and/or replacement of endogenous sequence.

A specific example of a nucleic acid encoding a catalytically inactive Cas protein can comprise, consist essentially of, or consist of a nucleic acid encoding an amino acid sequence at least 85%, 90%, 91%, 92%, 93%, 94%, 95%, 96%, 97%, 98%, 99%, or 100% identical to the dCas9 protein sequence set forth in SEQ ID NO: 2. Optionally, the nucleic acid can comprise, consist essentially of, or consist of a nucleic acid encoding an amino acid sequence at least 85%, 90%, 91%, 92%, 93%, 94%, 95%, 96%, 97%, 98%, 99%, or 100% identical to the sequence set forth in SEQ ID NO: 24 (optionally wherein the sequence encodes a protein at least 85%, 90%, 91%, 92%, 93%, 94%, 95%, 96%, 97%, 98%, 99%, or 100% identical to the dCas9 protein sequence set forth in SEQ ID NO: 2).

A specific example of a nucleic acid encoding a chimeric Cas protein can comprise, consist essentially of, or consist of a nucleic acid encoding an amino acid sequence at least 85%, 90%, 91%, 92%, 93%, 94%, 95%, 96%, 97%, 98%, 99%, or 100% identical to the chimeric Cas protein sequence set forth in SEQ ID NO: 1. Optionally, the nucleic acid can comprise, consist essentially of, or consist of a nucleic acid encoding an amino acid sequence at least 85%, 90%, 91%, 92%, 93%, 94%, 95%, 96%, 97%, 98%, 99%, or 100% identical to the sequence set forth in SEQ ID NO: 25 (optionally wherein the sequence encodes a protein at least 85%, 90%, 91%, 92%, 93%, 94%, 95%, 96%, 97%, 98%, 99%, or 100% identical to the chimeric Cas protein sequence set forth in SEQ ID NO: 1).

A specific example of a nucleic acid encoding an adaptor can comprise, consist essentially of, or consist of a nucleic acid encoding an amino acid sequence at least 85%, 90%, 91%, 92%, 93%, 94%, 95%, 96%, 97%, 98%, 99%, or 100% identical to MCP sequence set forth in SEQ ID NO: 7. Optionally, the nucleic acid can comprise, consist essentially of, or consist of a nucleic acid encoding an amino acid sequence at least 85%, 90%, 91%, 92%, 93%, 94%, 95%, 96%, 97%, 98%, 99%, or 100% identical to the sequence set forth in SEQ ID NO: 26 (optionally wherein the sequence encodes a protein at least 85%, 90%, 91%, 92%, 93%, 94%, 95%, 96%, 97%, 98%, 99%, or 100% identical to the MCP sequence set forth in SEQ ID NO: 7).

A specific example of a nucleic acid encoding a chimeric adaptor protein can comprise, consist essentially of, or consist of a nucleic acid encoding an amino acid sequence at least 85%, 90%, 91%, 92%, 93%, 94%, 95%, 96%, 97%, 98%, 99%, or 100% identical to the chimeric adaptor protein sequence set forth in SEQ ID NO: 6. Optionally, the nucleic acid can comprise, consist essentially of, or consist of a nucleic acid encoding an amino acid sequence at least 85%, 90%, 91%, 92%, 93%, 94%, 95%, 96%, 97%, 98%, 99%, or 100% identical to the sequence set forth in SEQ ID NO: 27 (optionally wherein the sequence encodes a protein at least 85%, 90%, 91%, 92%, 93%, 94%, 95%, 96%, 97%, 98%, 99%, or 100% identical to the chimeric adaptor protein sequence set forth in SEQ ID NO: 6).

Specific examples of nucleic acids encoding transcriptional activation domains can comprise, consist essentially of, or consist of a nucleic acid encoding an amino acid sequence at least 85%, 90%, 91%, 92%, 93%, 94%, 95%, 96%, 97%, 98%, 99%, or 100% identical to the VP64, p65, or HSF1 sequences set forth in SEQ ID NO: 3, 8, or 9, respectively. Optionally, the nucleic acid can comprise, consist essentially of, or consist of a nucleic acid encoding an amino acid sequence at least 85%, 90%, 91%, 92%, 93%, 94%, 95%, 96%, 97%, 98%, 99%, or 100% identical to the sequence set forth in SEQ ID NO: 28, 29, or 30, respectively (optionally wherein the sequence encodes a protein at least 85%, 90%, 91%, 92%, 93%, 94%, 95%, 96%, 97%, 98%, 99%, or 100% identical to the VP64, p65, or HSF1 sequences set forth in SEQ ID NO: 3, 8, or 9, respectively).

One example of a synergistic activation mediator (SAM) expression cassette comprises from 5′ to 3′: (a) a 3′ splicing sequence; (b) a first recombinase recognition site (e.g., loxP site); (c) a coding sequence for a drug resistance gene (e.g., neomycin phosphotransferase (neon) coding sequence); (d) a polyadenylation signal; (e) a second recombinase recognition site (e.g., loxP site); (f) a chimeric Cas protein coding sequence (e.g., dCas9-NLS-VP64 fusion protein); (g) a 2A protein coding sequence (e.g., a T2A coding sequence); and (e) a chimeric adaptor protein coding sequence (e.g., MCP-NLS-p65-HSF1). See, e.g., SEQ ID NO: 31 (coding sequence set forth in SEQ ID NO: 46 and encoding protein set forth in SEQ ID NO: 44, with the mRNA sequence set forth in SEQ ID NO: 61).

One example of a generic guide RNA array expression cassette comprises from 5′ to 3′: (a) a 3′ splicing sequence; (b) a first recombinase recognition site (e.g., rox site); (c) a coding sequence for a drug resistance gene (e.g., puromycin-N-acetyltransferase (puro^{r}) coding sequence); (d) a polyadenylation signal; (e) a second recombinase recognition site (e.g., rox site); (f) a guide RNA comprising one or more guide RNA genes (e.g., a first U6 promoter followed by a first guide RNA coding sequence, a second U6 promoter followed by a second guide RNA coding sequence, and a third U6 promoter followed by a third guide RNA coding sequence). See, e.g., SEQ ID NO: 32. The region of SEQ ID NO: 32 comprising the promoters and guide RNA coding sequences is set forth in SEQ ID NO: 47. Such a guide RNA array expression cassette encoding guide RNAs targeting mouse Ttr is set forth in SEQ ID NO: 33. The region of SEQ ID NO: 33 comprising the promoters and guide RNA coding sequences is set forth in SEQ ID NO: 48.

Another example of a generic guide RNA array expression cassette comprises one or more guide RNA genes (e.g., a first U6 promoter followed by a first guide RNA coding sequence, a second U6 promoter followed by a second guide RNA coding sequence, and a third U6 promoter followed by a third guide RNA coding sequence). Such a generic guide RNA array expression cassette is set forth in SEQ ID NO: 48. Examples of such guide RNA array expression cassettes for specific genes are set forth, e.g., in SEQ ID NOS: 33, 48, and 49.

### IV. Lipid Nanoparticles and Introducing Guide RNAs and Other Components into Cells and Eukaryotic Organisms

Also disclosed herein are lipid nanoparticles (LNPs) for delivering all of the SAM system components in the same LNP to a cell or eukaryotic organism in order to increase transcription or expression of a target gene. The methods disclosed herein comprise introducing into a cell or eukaryotic organism (e.g., animal, non-human animal, mammal, or non-human mammal) all of the components of a synergistic activation mediator (SAM) system (one or more guide RNAs or nucleic acids encoding, a chimeric Cas protein or nucleic acid encoding, and a chimeric adaptor protein or nucleic acid encoding) together in the same LNP. For example, such a LNP can comprise a cargo comprising: (a) a nucleic acid encoding a chimeric Clustered Regularly Interspaced Short Palindromic Repeats (CRISPR) associated (Cas) protein comprising a nuclease-inactive Cas protein fused to one or more transcriptional activation domains; (b) a nucleic acid encoding a chimeric adaptor protein comprising an adaptor fused to one or more transcriptional activation domains; and (c) one or more guide RNAs or one or more nucleic acids encoding the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within the target gene, thereby increasing expression of the target gene. In one example, all of the components of the synergistic activation mediator system are introduced in the form of RNA together in the same LNP. “Introducing” includes presenting to the cell or eukaryotic organism (e.g., animal, non-human animal, mammal, or non-human mammal) the nucleic acid or protein in such a manner that the nucleic acid or protein gains access to the interior of the cell or to the interior of cells within the eukaryotic organism (e.g., animal, non-human animal, mammal, or non-human mammal).

A guide RNA can be introduced into the cell in the form of an RNA (e.g., in vitro transcribed RNA) or in the form of a DNA encoding the guide RNA. Likewise, protein components such as chimeric Cas proteins and chimeric adaptor proteins can be introduced into the cell in the form of DNA, RNA, or protein. When introduced in the form of a DNA, the DNA encoding a guide RNA can be operably linked to a promoter active in the cell. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules). Nucleic acids encoding chimeric Cas proteins, chimeric adaptor proteins, or guide RNAs are discussed in more detail elsewhere herein.

In a specific example, the one or more guide RNAs, the chimeric Cas protein, and the chimeric adaptor protein are each introduced in the form of RNA via LNP-mediated delivery in the same LNP. As discussed in more detail elsewhere herein, one or more of the RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. Delivery through such methods results in transient Cas expression and/or presence of the guide RNA, and the biodegradable lipids improve clearance, improve tolerability, and decrease immunogenicity. Lipid formulations can protect biological molecules from degradation while improving their cellular uptake.

Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include a SAM mRNA and a guide RNA or a nucleic acid encoding a guide RNA.

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,127)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

In some LNPs, the cargo can comprise Cas mRNA or SAM mRNA (e.g., a bicistronic mRNA encoding both the chimeric Cas protein and the chimeric adaptor protein separated, for example, by a 2A-encoding sequence) and gRNA. The Cas mRNA/SAM mRNA and gRNAs can be in different ratios. For example, the LNP formulation can include a ratio of Cas mRNA/SAM mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA/SAM mRNA to gRNA nucleic acid from about 1:1 to about 1:5, or about 10:1. Alternatively, the LNP formulation can include a ratio of Cas mRNA/SAM mRNA to gRNA nucleic acid of about 1:10, 25:1, 10:1, 5:1, 3:1, 1:1, 1:3, 1:5, 1:10, or 1:25. Alternatively, the LNP formulation can include a ratio of Cas mRNA/SAM mRNA to gRNA nucleic acid of from about 1:1 to about 1:2. In specific examples, the ratio of Cas mRNA/SAM mRNA to gRNA can be about 1:1 or about 1:2.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In one example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used.

A specific example of a suitable LNP has a nitrogen-to-phosphate (N/P) ratio of 4.5 and contains biodegradable cationic lipid, cholesterol, DSPC, and PEG2k-DMG in a 45:44:9:2 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA/SAM mRNA can be in a 1:1 ratio by weight to the guide RNA. Another specific example of a suitable LNP contains Dlin-MC3-DMA (MC3), cholesterol, DSPC, and PEG-DMG in a 50:38.5:10:1.5 molar ratio.

Another specific example of a suitable LNP has a nitrogen-to-phosphate (N/P) ratio of 6 and contains biodegradable cationic lipid, cholesterol, DSPC, and PEG2k-DMG in a 50:38:9:3 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. The Cas9 mRNA/SAM mRNA can be in a 1:2 ratio by weight to the guide RNA.

Another specific example of a suitable LNP has a nitrogen-to-phosphate (N/P) ratio of 3 and contains a cationic lipid, a structural lipid, cholesterol (e.g., cholesterol (ovine) (Avanti 700000)), and PEG2k-DMG (e.g., PEG-DMG 2000 (NOF America-STJNBRIGHT® GM-020(DMG-PEG)) in a 50:10:38.5:1.5 ratio or a 47:10:42:1 ratio. The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)).

Another specific example of a suitable LNP contains Dlin-MC3-DMA, DSPC, cholesterol, and a PEG lipid in a 45:9:44:2 ratio. Another specific example of a suitable LNP contains Dlin-MC3-DMA, DOPE, cholesterol, and PEG lipid or PEG DMG in a 50:10:39:1 ratio. Another specific example of a suitable LNP has Dlin-MC3-DMA, DSPC, cholesterol, and PEG2k-DMG at a 55:10:32.5:2.5 ratio. Another specific example of a suitable LNP has Dlin-MC3-DMA, DSPC, cholesterol, and PEG-DMG in a 50:10:38.5:1.5 ratio. Another specific example of a suitable LNP has Dlin-MC3-DMA, DSPC, cholesterol, and PEG-DMG in a 50:10:38.5:1.5 ratio

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyms, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion. Nasal instillation and intravitreal injection are other specific examples. Compositions comprising the guide RNAs (or nucleic acids encoding the guide RNAs) can be formulated using one or more physiologically and pharmaceutically acceptable carriers, diluents, excipients or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can be depend, for example, on the half-life of the guide RNAs or chimeric Cas protein or chimeric adaptor protein mRNAs and the route of administration among other factors. The introduction of nucleic acids or proteins into the cell or eukaryotic organism (e.g., animal, non-human animal, mammal, or non-human mammal) can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. When a DNA sequence encoding an amino acid sequence is provided, it is understood that RNA sequences that encode the same amino acid sequence are also provided (by replacing the thymines with uracils). The amino acid sequences follow the standard convention of beginning at the amino terminus of the sequence and proceeding forward (i.e., from left to right in each line) to the carboxy terminus.

TABLE 4
Description of Sequences.

| SEQ ID | | |
|---|---|---|
| NO | Type | Description |
| 1 | Protein | dCas9-VP64 chimeric Cas protein |
| 2 | Protein | dCas9 protein |
| 3 | Protein | VP64 transcriptional activation domain |
| 4 | Protein | Linker v1 |
| 5 | Protein | Linker v2 |
| 6 | Protein | MCP-p65-HSF1 chimeric adaptor protein |
| 7 | Protein | MS2 coat protein (MCP) |
| 8 | Protein | p65 transcriptional activation domain |
| 9 | Protein | HSF1 transcriptional activation domain |
| 10 | RNA | crRNA tail |
| 11 | RNA | tracrRNA |
| 12 | RNA | gRNA scaffold v1 |
| 13 | RNA | gRNA scaffold v2 |
| 14 | RNA | gRNA scaffold v3 |
| 15 | RNA | gRNA scaffold v4 |
| 16 | RNA | MS2-binding loop |
| 17 | DNA | Guide RNA target sequence plus PAM v1 |
| 18 | DNA | Guide RNA target sequence plus PAM v2 |
| 19 | DNA | Guide RNA target sequence plus PAM v3 |
| 20 | Protein | T2A |
| 21 | Protein | P2A |
| 22 | Protein | E2A |
| 23 | Protein | F2A |
| 24 | DNA | Nucleic acid encoding dCas9 protein |
| 25 | DNA | Nucleic acid encoding dCas9-VP64 chimeric Cas |
| | | protein |
| 26 | DNA | Nucleic acid encoding MCP |
| 27 | DNA | Nucleic acid encoding MCP-p65-HSF1 chimeric |
| | | adaptor protein |
| 28 | DNA | Nucleic acid encoding VP64 transcriptional activation |
| | | domain |
| 29 | DNA | Nucleic acid encoding p65 transcriptional activation |
| | | domain |
| 30 | DNA | Nucleic acid encoding HSF1 transcriptional activation |
| | | domain |
| 31 | DNA | Synergistic activation mediator (SAM) bicistronic |
| | | expression cassette (dCas9-VP64-T2A-MCP-p65- |
| | | HSF1) |
| 32 | DNA | Generic guide RNA array expression cassette |
| 33 | DNA | Ttr guide RNA array expression cassette |
| 34 | DNA | Mouse Ttr guide RNA target sequence v1 |
| 35 | DNA | Mouse Ttr guide RNA target sequence v2 |
| 36 | DNA | Mouse Ttr guide RNA target sequence v3 |
| 37 | RNA | Mouse Ttr single guide RNA v1 |
| 38 | RNA | Mouse Ttr single guide RNA v2 |
| 39 | RNA | Mouse Ttr single guide RNA v3 |
| 40 | RNA | gRNA scaffold with MS2 binding loops |
| 41 | RNA | Mouse Ttr guide RNA DNA-targeting segment v1 |
| 42 | RNA | Mouse Ttr guide RNA DNA-targeting segment v2 |
| 43 | RNA | Mouse Ttr guide RNA DNA-targeting segment v3 |
| 44 | Protein | Synergistic activation mediator (SAM) (dCas9-VP64- |
| | | T2A-MCP-p65-HSF1) |
| 45 | RNA | Generic single gRNA with MS2 binding loops |
| 46 | DNA | Synergistic activation mediator (SAM) coding |
| | | sequence (dCas9-VP64-T2A-MCP-p65-HSF1) |
| 47 | DNA | Generic guide RNA array promoters and guide RNA |
| | | coding sequences |
| 48 | DNA | Ttr guide RNA array promoters and guide RNA |
| | | coding sequences |
| 49 | DNA | pscAAV Ttr array |
| 50 | RNA | tracrRNA v2 |
| 51 | RNA | tracrRNA v3 |
| 52 | RNA | gRNA scaffold v5 |
| 53 | RNA | gRNA scaffold v6 |
| 54 | RNA | gRNA scaffold v7 |
| 55 | RNA | Mouse Ttr single guide RNA v3b |
| 56 | RNA | gRNA scaffold with MS2 binding loops v2 |
| 57 | RNA | Generic single gRNA with MS2 binding loops v2 |
| 58 | Protein | SV40 NLS v1 |
| 59 | Protein | SV40 NLS v2 |
| 60 | Protein | NLS of nucleoplasmin |
| 61 | RNA | Synergistic activation mediator (SAM) mRNA |
| | | (dCas9-VP64-T2A-MCP-p65-HSF1) |

### EXAMPLES

### Example 1. LNP-Mediated dCas9 SAM Delivery

In this example, we focused on up-regulating gene expression using the dCas9 (catalytically dead Cas9) synergistic activation mediator (SAM) system. In this system, several activation domains interact to cause a greater gene response than could be induced by any one factor alone. The components include: (1) dCas9 directly fused to a VP64 domain, a transcriptional activator composed of four tandem copies of Herpes Simplex Viral Protein 16; (2) MS2 coat protein (MCP) fused to two additional activating transcription factors: heat-shock factor 1 (HSF1) and transcription factor 65 (p65); and (3) MS2-loop-containing sgRNA (increased in length from ˜97 nucleotides to ˜166 nucleotides with the inclusion of the MS2-binding loops). When VP64 is fused to a protein that binds near a transcriptional start site, it acts as a strong transcriptional activator. The MCP naturally binds to MS2 stem loops. In this system, MCP interacts with MS2 stem loops engineered into the CRISPR associated sgRNA and thereby shuttles the bound transcription factors to the appropriate genomic location.

The initial iteration of this system used three separate lentiviruses to deliver the three separate components. While the three-component system allows for some flexibility in cell culture, this set-up is less desirable in an animal model. Instead, we first chose to introduce the dCas9, VP64, MCP, HSF1, and p65 as one transcript driven by the murine Rosa26 promoter. We could then introduce guide RNAs by recombinant adeno-associated virus (AAV) injection into the mouse tail vein for liver-specific upregulation. WT AAVs are generally considered safe for gene therapy as they have low immunogenicity and have a highly predictable integration site (AAVS1 on human chromosome 19). However, to increase their safety as gene therapy vectors, the integrative capacity of the WT AAVs has been eliminated such that these vectors remain as episomes in the host cell nucleus. For the purposes of this example, all AAV references indicate the recombinant variant. Upon the introduction to a host, the immune response against the AAV is generally restricted to neutralizing antibodies with no clearly defined cytotoxic response. In non-dividing cells, these AAV episomes remain intact for the life of the host cell. In dividing cells, the AAV DNA is diluted out through cell division, making it necessary to administer more virus for continued therapeutic response. These subsequent exposures may result in rapid neutralization of the virus and, therefore, a decreased host response. To get around this, researchers will use alternative serotypes for sequential infections, though this is hampered by serotype specificity.

Another concern in AAV-based therapeutics is the relatively small cloning capacity: 4.6 kb between the two inverted terminal repeats. As the complete coding sequence of dCas9 SAM is ˜5.8 kb (without a promoter), we cannot express all components from a single AAV. One method to get around this is to work in a dCas9 SAM mouse background in which the mouse comprises a dCas9-NLS-VP64-T2A-MCP-NLS-p65-HSF1 expression cassette (SAM expression cassette) genomically integrated into the first intron of the Rosa26 locus such that the mice express all components of the SAM system except for the gRNAs. See, e.g., U.S. patent application Ser. No. 16/358,395 filed Mar. 19, 2019, and PCT Patent Application No. PCT/US2019/023009 filed Mar. 19, 2019, each of which is herein incorporated by reference in its entirety for all purposes. In these mice, the S. pyogenes dCas9 coding sequence (CDS) in the expression cassette was codon-optimized for expression in mice. The encoded dCas9 includes the following mutations to render the Cas9 nuclease-inactive: D10A and N863A. The dCas9-NLS-VP64-T2A-MCP-NLS-p65-HSF1 expression cassette (SAM expression cassette) is set forth SEQ ID NO: 31. The synergistic activation mediator (SAM) coding sequence (dCas9-VP64-T2A-MCP-p65-HSF1) is set forth in SEQ ID NO: 46 and encodes the protein set forth in SEQ ID NO: 44. The synergistic activation mediator (SAM) mRNA sequence (dCas9-VP64-T2A-MCP-p65-HSF1) is set forth in SEQ ID NO: 61. The expression cassette was targeted to the first intron of the Rosa26 locus to take advantage of the strong universal expression of the Rosa26 locus and the ease of targeting the Rosa26 locus.

However, for obvious reasons, using a dCas9-SAM-expressing mouse is not an option in a clinical setting. Alternatively, one could express the elements across two or more AAVs and hope that they both infect the same cell. Again, this is less than desirable for a therapeutic solution. With this in mind, we set out to optimize this system such that it can have a clinical translation.

Lipid nanoparticles (LNPs) make an attractive alternative to AAV use as they safely and effectively deliver nucleic acids to cells by leveraging the endogenous endocytosis mechanism to bring the molecules in via LDL receptors. Variation in the formulation can influence the particle's stability and tropism once introduced into an organism. Furthermore, conjugation of various ligands can further increase target specificity of the LNP. One caveat to this delivery method is the transient effect on host cells, as wild type Cas9 mRNA delivered to hepatocytes by LNP can be cleared within a few days of cellular intake in some cases (data not shown). However, there is no immune response to LNP delivery, which allows for well-tolerated sequential dosing. Unfortunately, the application of this delivery system to dCas9 SAM gene activation has been limited by limitations in RNA synthesis technologies. These limitations have precluded the generation of SAM sgRNAs with stabilizing end modifications, as these molecules are greater than the 110-nucleotide platform maximum. Recently, however, RNA synthesis technologies have increased their capacity to 200 synthetic nucleotides, with end modifications, allowing us to evaluate LNP delivery of SAM sgRNA.

With the goal of creating an amyloidosis study model, we tested delivery of SAM gRNAs directed to transthyretin (Ttr). Wild type TTR can dissociate, misfold and aggregate, leading to disease-inducing amyloid build-up.

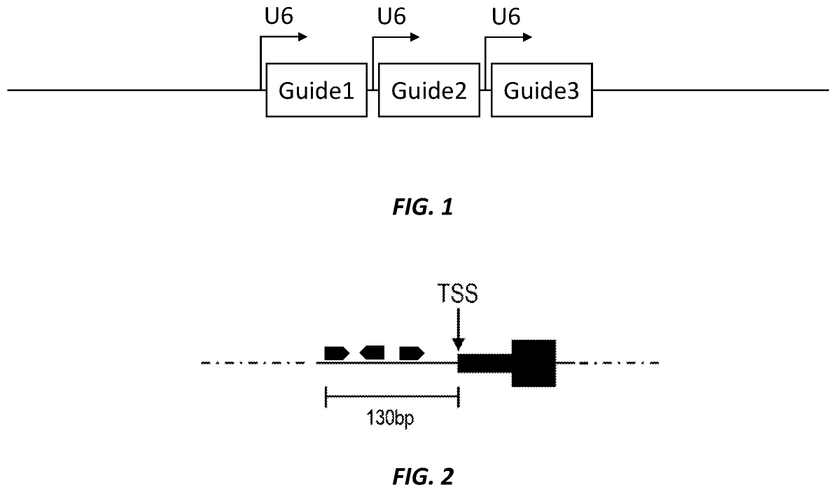

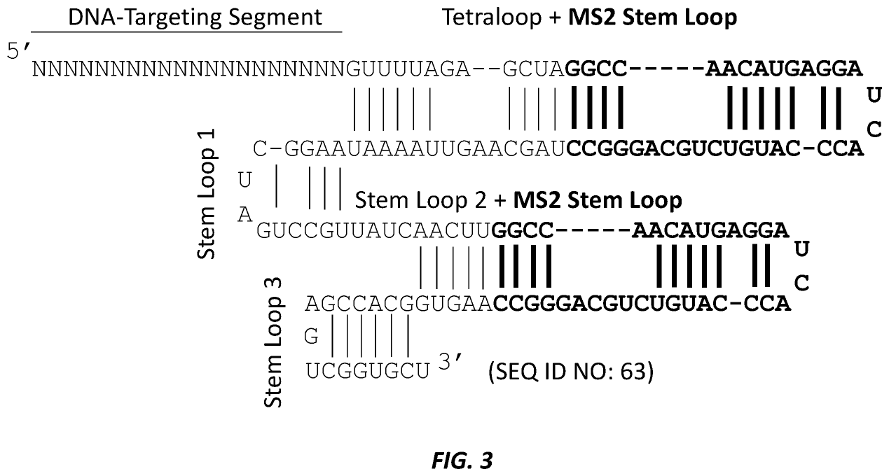

We precisely overexpressed the Ttr gene by tail-vein injection of a liver-specific AAV (serotype 8) expressing an array of three Ttr SAM guides. The Ttr guide RNA array is depicted in FIG. 1 and in SEQ ID NO: 33. The region including the promoters and guide RNA coding sequences is set forth in SEQ ID NO: 48. The guide RNA target sequences (not including PAM) in the mouse Ttr gene that are targeted by the guide RNAs in the array are set forth in SEQ ID NO: 34 (ACGGTTGCCCTCTTTCCCAA), SEQ ID NO: 35 (ACTGTCAGACTCAAAGGTGC), and SEQ ID NO: 36 (GACAATAAGTAGTCTTACTC), respectively. SEQ ID NO: 34 (Ttr gA) is located −63 of the Ttr transcription start site, SEQ ID NO: 35 (Ttr gA2) is located −134 of the Ttr transcription start site, and SEQ ID NO: 36 (Ttr gA3) is located −112 of the Ttr transcription start site. The single guide RNAs targeting these guide RNA target sequences are set forth in SEQ ID NOS: 37, 38, and 39, respectively. The guides were designed to direct the dCas9 SAM components to the 100-200 bp region upstream of the Ttr transcriptional start site (TSS). See FIG. 2. A general schematic of the structure of each guide RNA, including the MS2 stem loops, is shown in FIG. 3 (SEQ ID NO: 45).

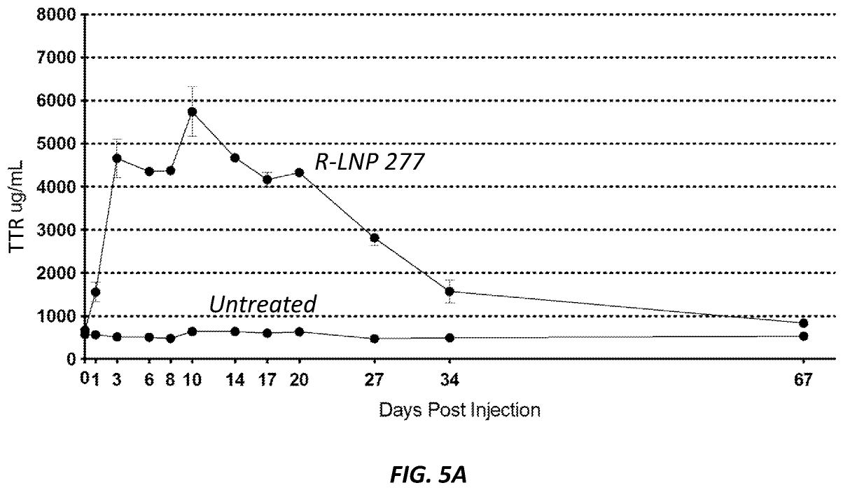

Three groups of mice were assessed: (1) Rosa26-dCas9-SAM (untreated); (2) Rosa26-dCas9-SAM (AAV8-GFP); and (3) Rosa26-dCas9-SAM (AAV8-gTTR array (three guides targeting Ttr)). These mice were injected with AAV8-GFP or AAV8-gTTR array at eight weeks of age and were followed out to eight months post-injection. The serum quantity of TTR was measured by ELISA at various early time points and then monthly, and these animals were observed for any pathological changes. While no pathologic changes were observed in these animals at eight months post-injection, they had an initial increase in circulating TTR of 11× by day 19, with levels finding a steady state of elevated TTR of ˜4× by five months post-injection. As shown in FIG. 4, dCas9 SAM mice treated with an unrelated virus maintain approximately 1000 μg/mL of circulating TTR, similar to WT mice. Meanwhile, the circulating TTR protein level of dCas9 SAM mice dosed with the AAV expressing SAM guide array spiked to 11,000 μg/mL by day 19. See FIG. 4. This level slowly decreased overtime as the virus particles were neutralized or the natural homeostasis was recovered. See FIG. 4. Either way, the circulating TTR protein levels are expected to drop to near wild type within a year without the ability to re-dose the study mice.

While LNP upregulation is expected to last for a significantly shorter time, the benefit of re-dosing can overcome this limitation. With that in mind, we endeavored to characterize how long protein elevation could be maintained from a single LNP delivery of a single SAM Ttr sgRNA. Two groups of mice were assessed: (1) Rosa26-dCas9-SAM (untreated); and (2) Rosa26-dCas9-SAM (R-LNP277-gTTR (one guide targeting Ttr)). The guide RNA target sequence (not including PAM) in the mouse Ttr gene that was targeted by the guide RNA is set forth in SEQ ID NO: 36 (GACAATAAGTAGTCTTACTC). The single guide RNA targeting this guide RNA target sequence is set forth in SEQ ID NO: 55. The single guide RNA was modified to include 2′-O-methyl analogs and 3′ phosphorothioate internucleotide linkages at the first three 5′ and last three 3′ residues.

For the LNP formulation, stock solutions of (6Z, 9Z, 28Z, 31Z)-heptatriaconta-6,9,28,31-tetraen-19yl 4-(dimethylamino)butanoate (MC3; Biofine), 1,2-distearoyl-sn-glycero-3-phosphocholine (DSPC; Avanti), cholesterol (Chol; Avanti), and 1,2-Dimyristoyl-sn-glycero methoxypolyethylene glycol (PEG-DMG (2000); NOF) at 50 mM in ethanol were used. These lipids were mixed to yield a molar ratio of 50:10:38.5:1.5 (MC3:DSPC:Chol:PEG-DMG). The gRNA was prepared in 10 mM sodium citrate (pH 5) to 225 μg/mL. Through the use of microfluidic mixing of the BenchTop Nanoassemblr (Precision Nanosystems), the RNA and lipids were mixed at 12 mL/min flow rate and at a 3:1 volumetric ratio of RNA:lipids. LNPs were diluted into PBS (pH 7.4) to dilute the ethanol and were subsequently concentrated using a centrifugal filter (Amicon, 10 kD cutoff). The RNA was quantified through a modified Ribrogreen assay (Life Technologies), and the LNPs were quantified in TE and TE with 2% Triton X-100. The total encapsulated RNA was determined by the measurement of RNA in the Triton-X100 sample (Total RNA)—TE sample (free RNA). Prior to delivery to animals, the LNPs were filtered through a 0.22 μm syringe filter and diluted to the appropriate concentration in PBS (pH 7.4) at a total volume for i.v. injection of 200 μL.

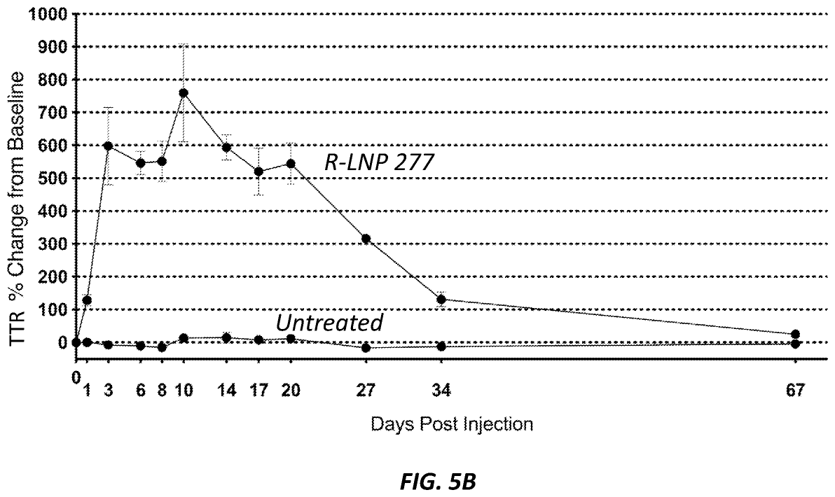

Three mice were tested in group (1), and two mice were tested in group (2). These mice were injected with 1 mpk LNP in 200 μL PBS at twelve weeks of age and were followed out to 67 days post-injection. The serum quantity of TTR was measured by ELISA at various early time points and then monthly. Surprisingly, increased TTR protein levels of 4,000 μg/mL were sustained at a constant level for several weeks, indicating that the upregulation was far less transient than anticipated. See FIGS. 5A and 5B. Of note, while the initial spike in protein associated with AAV delivery is much higher, our LNP delivery included only one of the three SAM Ttr sgRNAs. Furthermore, the TTR upregulation achieved by LNP delivery remained at a fairly constant level for several weeks, whereas the upregulation achieved by AAV delivery induction was highly unstable (initially drastically increasing over 19 days and then constantly dropping over time). AAV delivery induced a strong initial upregulation and allowed for expression over a year, but it continued to drop over time. LNP delivery allowed for a rapid increase in expression and was stable for several weeks, after which protein levels may return to normal if a subsequent dose is not provided.

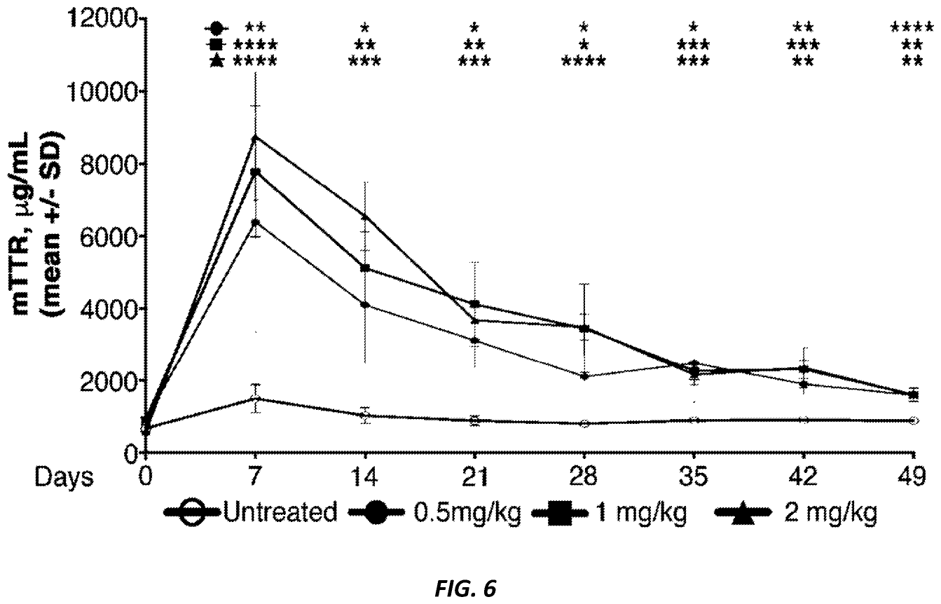

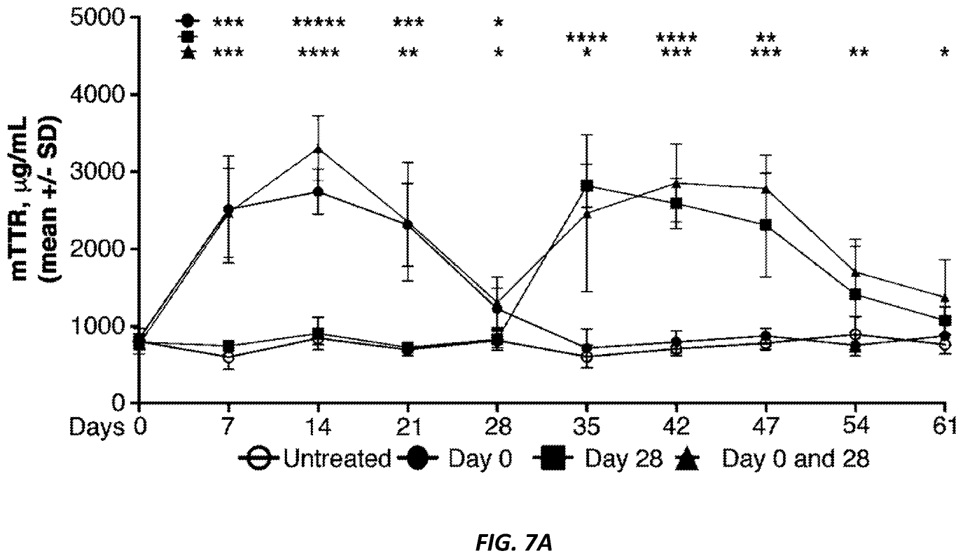

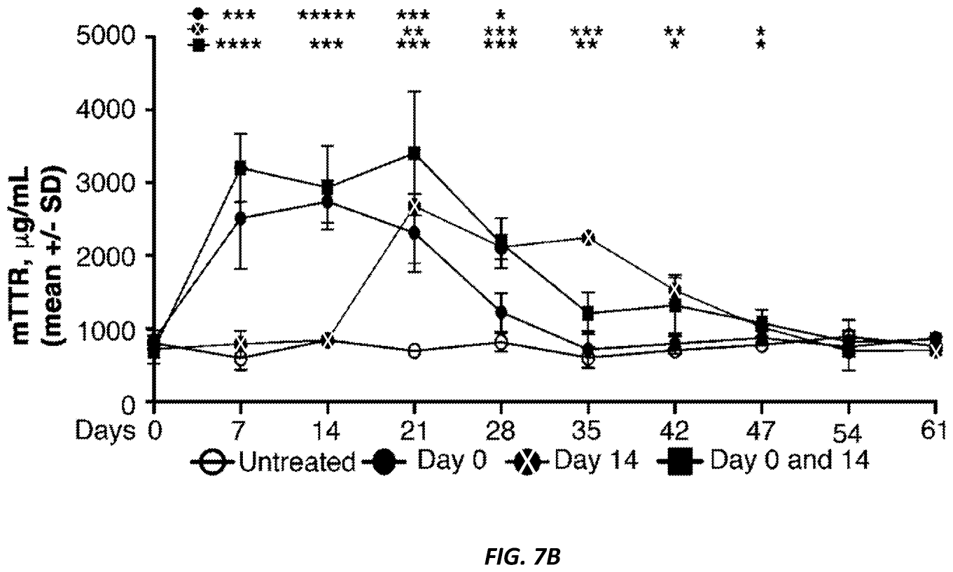

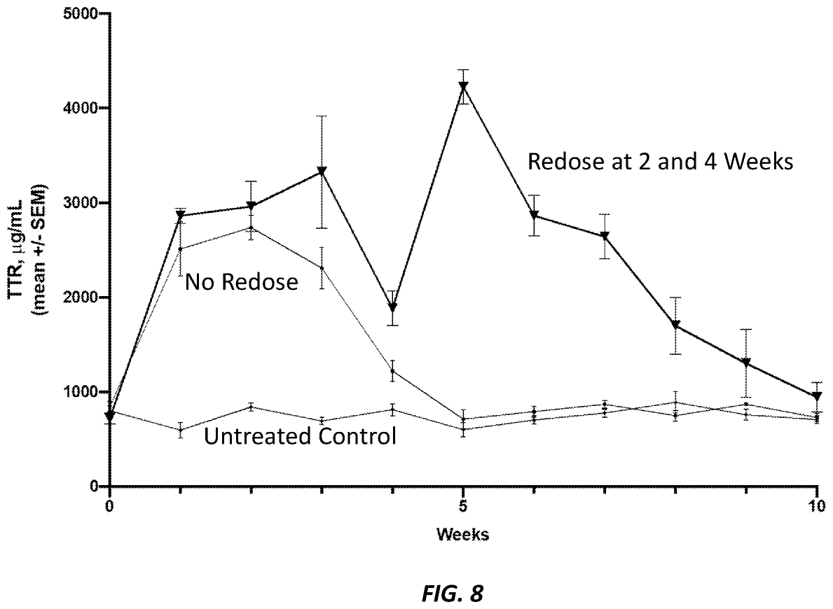

We next assessed the effect of administering different doses of LNP. A single SAM Ttr sgRNA (Ttr gA3) as in the experiment above was introduced into male Rosa26-dCas9-SAM mice by LNP at three doses: 0.5 milligrams per kilogram of mouse body weight (mpk), 1 mpk, and 2 mpk. LNP was injected via the tail vein to characterize how long protein elevation could be maintained from a single dose of LNP. This transient delivery method produced dose-dependent gene activation for approximately three weeks and elevated serum TTR levels for more than a month. In addition, a dose-dependent elevation was observed by ELISA. The lowest dose yielded a seven-fold increased, while the highest dose yielded a 15-fold increase. See FIG. 6. A second study was conducted to evaluate the impact of sequential dosing. All mice were injected with 0.5 mpk of LNP formulated with Ttr gA2 at the start of the study, and blood draws were taken weekly. Subsets of these mice were dosed with another 0.5 mpk LNP at two weeks or four weeks, with additional naïve mice injected to confirm LNP function (FIGS. 7A and 7B). In all cases, redosing successfully boosted Ttr expression. The successful redosing of animals at a single timepoint without adverse effects suggests that sequential dosing of the same animals may be viable. One additional study was performed in which mice were dosed 3 times: 0.5 mpk LNP formulated with Ttr gA2 at day 0, again with 0.5 mpk at 2 weeks, and a final dose of 0.5 mpk at 4 weeks. We observed a sustained upregulation of TTR of more than 2-fold for 7 weeks. See FIG. 8.

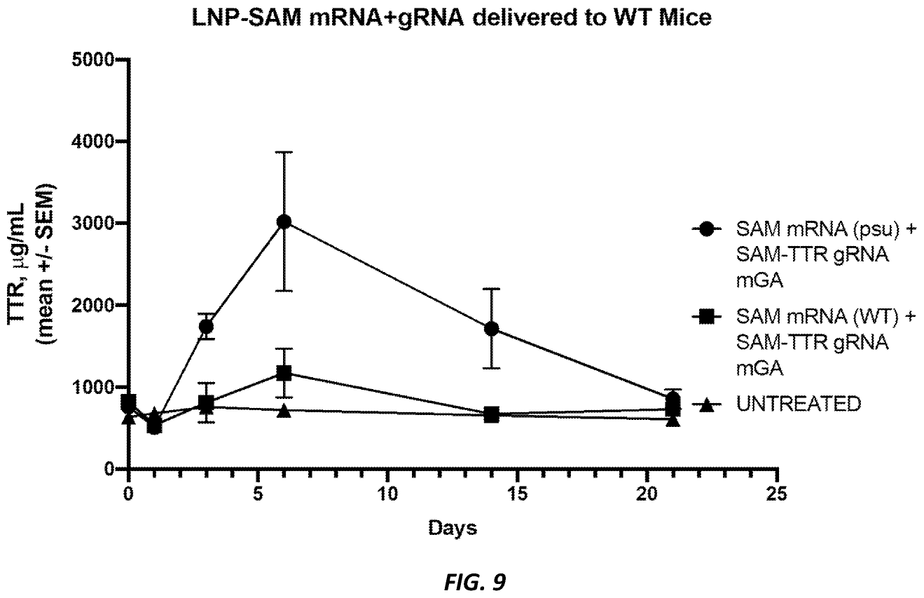

A single LNP formulated to include a cargo including both an in vitro transcribed mRNA encoding a synergistic activation mediator (dCas9-VP64-T2A-MCP-p65-HSF1) and a chemically synthesized SAM sgRNA targeting Ttr. The mRNA capped and polyadenylated and was either unmodified or pseudouridine (psu) modified (all standard uracil residues were replaced with pseudouridine, a uridine isomer in which the uracil is attached with a carbon-carbon bond rather than nitrogen-carbon). The SAM gRNA was modified to include 2′-O-methyl analogs and 3′ phosphorothioate internucleotide linkages at the first three 5′ and 3′ terminal RNA residues. Wild type mice were injected with LNP containing the modified mRNA and the SAM sgRNA targeting Ttr (Ttr gA2) or LNP containing the unmodified mRNA and the SAM sgRNA at 2 mpk at day 0, and serum levels of TTR were measured over 21 days. Untreated wild type mice were used as a negative control. As shown in FIG. 9, LNP containing the modified mRNA and the SAM sgRNA targeting Ttr successfully increased TTR serum levels from below 1000 μg/mL to more than 3000 μg/mL by Day 6, and LNP containing the unmodified mRNA and the SAM sgRNA targeting Ttr increased TTR serum levels to a lesser extent. This upregulation by a single dose persisted for at least 2 weeks.

A single LNP formulated as above is then generated to include a cargo including both an in vitro transcribed mRNA encoding a synergistic activation mediator (dCas9-VP64-T2A-MCP-p65-HSF1) and multiple chemically synthesized SAM sgRNAs targeting the same gene. The mRNA is polyadenylated and capped (TriLink CLEANCAP®), and the sgRNAs are modified to include 2′-O-methyl analogs and 3′ phosphorothioate internucleotide linkages at the first three 5′ and 3′ terminal RNA residues. Wild type mice are injected with the LNP, and expression of the target gene targeted by the SAM sgRNA is assessed.

LNP delivery of SAM sgRNA together with all of the other SAM components is a significant enhancement to therapeutic dCas9 SAM applications as we can now (1) ensure that the dCas9 SAM transcript and SAM sgRNA land in the same cell, (2) mediate increased tissue specificity with formulations/ligand incorporations, (3) re-dose organisms without fear of immune response, and (4) generate more stable expression levels. Taken together, this combination of nucleic acid delivery has greatly enhanced the potential dCas9 applications in a safe and unexpectedly stable manner.

1. A lipid nanoparticle for delivering a cargo to a target gene to increase expression of the target gene in an animal or cell, wherein the cargo comprises:
(a) a nucleic acid encoding a chimeric Clustered Regularly Interspaced Short Palindromic Repeats (CRISPR) associated (Cas) protein comprising a nuclease-inactive Cas protein fused to one or more transcriptional activation domains;
(b) a nucleic acid encoding a chimeric adaptor protein comprising an adaptor protein fused to one or more transcriptional activation domains; and
(c) one or more guide RNAs or one or more nucleic acids encoding the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within the target gene, thereby increasing expression of the target gene.

2.-57. (canceled)

58. A method for increasing expression of a target gene in an animal in vivo, comprising introducing into the animal:
(a) a nucleic acid encoding a chimeric Clustered Regularly Interspaced Short Palindromic Repeats (CRISPR) associated (Cas) protein comprising a nuclease-inactive Cas protein fused to one or more transcriptional activation domains;
(b) a nucleic acid encoding a chimeric adaptor protein comprising an adaptor protein fused to one or more transcriptional activation domains; and
(c) one or more guide RNAs or one or more nucleic acids encoding the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within the target gene, thereby increasing expression of the target gene,
wherein (a), (b), and (c) are delivered together in the same lipid nanoparticle (LNP).

59. The method of claim 58, wherein a multicistronic or bicistronic nucleic acid comprises (a) and (b).

60. The method of claim 59, wherein (a) and (b) are linked by a 2A protein coding sequence in the multicistronic or bicistronic nucleic acid.

61. The method of claim 58, wherein (a) and (b) are separate nucleic acids.

62. The method of claim 58, wherein (a) and (b) are each introduced in the form of a messenger RNA (mRNA).

63. The method of claim 62, wherein the mRNA is modified to be fully substituted with pseudouridine.

64. The method of claim 62, wherein the mRNA is a multicistronic or bicistronic nucleic acid comprising (a) and (b), wherein the mRNA comprises the sequence set forth in SEQ ID NO: 61.

65. The method of claim 58, wherein (c) is introduced in the form of RNA.

66. The method of claim 65, wherein each of the one or more guide RNAs is modified to comprise one or more stabilizing end modifications at the 5′ end and/or the 3′ end.

67. The method of claim 66, wherein the 5′ end and/or the 3′ end of each of the one or more guide RNAs is modified to comprise one or more phosphorothioate linkages.

68. The method of claim 66, wherein the 5′ end and/or the 3′ end of each of the one or more guide RNAs is modified to comprise one or more 2′-O-methyl modifications.

69. The method of claim 58, wherein the target sequence comprises a regulatory sequence within the target gene.

70. The method of claim 69, wherein the regulatory sequence comprises a promoter or an enhancer.

71. The method of claim 58, wherein the target sequence is within 200 base pairs of the transcription start site of the target gene.

72. The method of claim 71, wherein the target sequence is within the region 200 base pairs upstream of the transcription start site and 1 base pair downstream of the transcription start site.

73. The method of claim 58, wherein each of the one or guide RNAs comprises two adaptor-binding elements to which the chimeric adaptor protein can specifically bind.

74. The method of claim 73, wherein a first adaptor-binding element is within a first loop of each of the one or more guide RNAs, and a second adaptor-binding element is within a second loop of each of the one or more guide RNAs.

75. The method of claim 74, wherein each of the one or more guide RNAs is a single guide RNA comprising a CRISPR RNA (crRNA) portion fused to a transactivating CRISPR RNA (tracrRNA) portion, and
wherein the first loop is the tetraloop corresponding to residues 13-16 of SEQ ID NO: 12, 14, 52, or 53, and the second loop is the stem loop 2 corresponding to residues 53-56 of SEQ ID NO: 12, 14, 52, or 53.

76. The method of claim 58, wherein the adaptor-binding element comprises the sequence set forth in SEQ ID NO: 16.

77. The method of claim 76, wherein each of the one or more guide RNAs comprises the sequence set forth in SEQ ID NO: 40, 45, 56, or 57.

78. The method of claim 58, wherein at least one of the one or more guide RNAs targets a Ttr gene, optionally wherein the Ttr-targeting guide RNA targets a sequence comprising the sequence set forth in any one of SEQ ID NOS: 34-36 or optionally wherein the Ttr-targeting guide RNA comprises the sequence set forth in any one of SEQ ID NOS: 37-39 and 55.

79. The method of claim 58, wherein the one or more guide RNAs target two or more target genes.

80. The method of claim 58, wherein the one or more guide RNAs comprise multiple guide RNAs that target a single target gene.

81. The method of claim 58, wherein the one or more guide RNAs comprise at least three guide RNAs that target a single target gene.

82. The method of claim 81, wherein the at least three guide RNAs target the mouse Ttr locus, and wherein a first guide RNA targets a sequence comprising SEQ ID NO: 34 or comprises the sequence set forth in SEQ ID NO: 37, a second guide RNA targets a sequence comprising SEQ ID NO: 35 or comprises the sequence set forth in SEQ ID NO: 38, and a third guide RNA targets a sequence comprising SEQ ID NO: 36 or comprises the sequence set forth in SEQ ID NO: 39 or 55.

83. The method of claim 58, wherein the Cas protein is a Cas9 protein.

84. The method of claim 83, wherein the Cas9 protein is a Streptococcus pyogenes Cas9 protein, a Campylobacter jejuni Cas9 protein, or a Staphylococcus aureus Cas9 protein.

85. The method of claim 83, wherein the Cas9 protein comprises mutations corresponding to D10A and N863A or D10A and H840A when optimally aligned with a Streptococcus pyogenes Cas9 protein.

86. The method of claim 58, wherein the sequence encoding the Cas protein is codon-optimized for expression in the animal.

87. The method of claim 58, wherein the one or more transcriptional activator domains in the chimeric Cas protein are selected from: VP16, VP64, p65, MyoD1, HSF1, RTA, SETT/9, and a combination thereof.

88. The method of claim 87, wherein the one or more transcriptional activator domains in the chimeric Cas protein comprise VP64.

89. The method of claim 88, wherein the chimeric Cas protein comprises from N-terminus to C-terminus: the catalytically inactive Cas protein; a nuclear localization signal; and the VP64 transcriptional activator domain.

90. The method of claim 89, wherein the chimeric Cas protein comprises a sequence at least 90%, 95%, 96%, 97%, 98%, 99%, or 100% identical to the sequence set forth in SEQ ID NO: 1.

91. The method of claim 90, wherein the nucleic acid encoding the chimeric Cas protein comprises a sequence at least 90%, 95%, 96%, 97%, 98%, 99%, or 100% identical to the sequence set forth in SEQ ID NO: 25.

92. The method of claim 58, wherein the adaptor protein is at the N-terminal end of the chimeric adaptor protein, and the one or more transcriptional activation domains are at the C-terminal end of the chimeric adaptor protein.

93. The method of claim 58, wherein the adaptor protein comprises an MS2 coat protein or a functional fragment or variant thereof.

94. The method of claim 58, wherein the one or more transcriptional activation domains in the chimeric adaptor protein are selected from: VP16, VP64, p65, MyoD1, HSF1, RTA, SETT/9, and a combination thereof.

95. The method of claim 94, wherein the one or more transcriptional activation domains in the chimeric adaptor protein comprise p65 and HSF1.

96. The method of claim 95, wherein the chimeric adaptor protein comprises from N-terminus to C-terminus: an MS2 coat protein; a nuclear localization signal; the p65 transcriptional activation domain; and the HSF1 transcriptional activation domain.

97. The method of claim 96, wherein the chimeric adaptor protein comprises a sequence at least 90%, 95%, 96%, 97%, 98%, 99%, or 100% identical to the sequence set forth in SEQ ID NO: 6.

98. The method of claim 97, wherein the nucleic acid encoding the chimeric adaptor protein comprises a sequence at least 90%, 95%, 96%, 97%, 98%, 99%, or 100% identical to the sequence set forth in SEQ ID NO: 27.

99. The method of claim 58, wherein the animal is a non-human animal.

100. The method of claim 58, wherein the animal is a mammal.

101. The method of claim 100, wherein the mammal is a rodent.

102. The method of claim 101, wherein the rodent is a rat or a mouse.

103. The method of claim 102, wherein the rodent is the mouse.

104. The method of claim 58, wherein the animal is a human.

105. The method of claim 58, wherein the animal is a subject in need of increased expression of the target gene, wherein the target gene is underexpressed in the subject, and the underexpression is associated with or causative of a disease, disorder, or syndrome in the subject.

106. The method of claim 58, wherein the target gene is a gene expressed in the liver.

107. The method of claim 58, wherein the target gene is a disease-associated gene.

108. The method of claim 58, wherein decreased expression or activity of the target gene is associated with or causative of a disease, disorder, or syndrome.

109. The method of claim 58, wherein the target gene is a haploinsufficient gene or is OTC, HBG1, or HBG2.

110. The method of claim 109, wherein the target gene is a haploinsufficient gene selected from the genes listed in Table 3.

111. The method of claim 109, wherein the haploinsufficient gene is KCNQ4, PINK1, TP73, GLUT1, MYH, ABCA4, LRH-1, PAX8, SLC40A1, BMPR2, PKD2, PIK3R1, HMGA1, GCK, ELN, GTF3, GATA3, BUB3, PAX6, FLI1, HNF1A, PKD1, MC4R, DMPK, or MYH9.

112. The method of claim 58, wherein increased expression or activity of the target gene is associated with or causative of a disease, disorder, or syndrome.

113. The method of claim 58, wherein the lipid nanoparticle comprises a cationic lipid, a neutral lipid, a helper lipid, and a stealth lipid.

114. The method of claim 113, wherein the cationic lipid is MC3 and/or the neutral lipid is DSPC and/or the helper lipid is cholesterol and/or the stealth lipid is PEG-DMG.

115. The method of claim 114, wherein the lipid nanoparticle comprises MC3, DSPC, cholesterol, and PEG-DMG in a molar ratio of about 50:10:38.5:1.5.

116. The method of claim 58, wherein the route of administration of the one or more guide RNAs to the animal is intravenous injection, intraparenchymal injection, intraperitoneal injection, nasal installation, or intravitreal injection.

117. The method of claim 58, wherein the increase in expression of the target gene is at least 0.5-fold, 1-fold, 2-fold, 3-fold, 4-fold, 5-fold, 6-fold, 7-fold, 8-fold, 9-fold, 10-fold, or 20-fold higher relative to a control animal.

118. The method of claim 58, wherein the duration of the increase in expression of the target gene is at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, or at least about 2 months.

119. The method of claim 58, wherein the lipid nanoparticle comprising (a), (b), and (c) is introduced into the animal two or more times sequentially.

120. The method of claim 119, wherein the lipid nanoparticle comprising (a), (b), and (c) is introduced into the animal three or more times sequentially.

121. The method of claim 119, wherein expression of the target gene is increased to at least the same level after each sequential introduction of the lipid nanoparticle.

122. The method of claim 119, wherein expression of the target gene is increased to a higher level than in methods in which the lipid nanoparticle is introduced only once.

123. A method for increasing expression of a target gene in a cell, comprising introducing into the cell:
(a) a nucleic acid encoding a chimeric Clustered Regularly Interspaced Short Palindromic Repeats (CRISPR) associated (Cas) protein comprising a nuclease-inactive Cas protein fused to one or more transcriptional activation domains;
(b) a nucleic acid encoding a chimeric adaptor protein comprising an adaptor protein fused to one or more transcriptional activation domains; and
(c) one or more guide RNAs or one or more nucleic acids encoding the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within the target gene, thereby increasing expression of the target gene,
wherein (a), (b), and (c) are delivered together in the same lipid nanoparticle (LNP).
